(19) United States
(12) Patent Application Publication
(10) Pub. No.: US 2024/0108687 A1
(43) Pub. Date: Apr. 4, 2024

(54) COMPOSITIONS AND METHODS FOR USING COMBINATIONS OF ACTIN-BASED PEPTIDES TO MODULATE CELLULAR BIOACTIVITY

(71) Applicant: Virongy L.L.C., Manassas, VA (US)

(72) Inventor: Yuntao WU, Manassas, VA (US)

(73) Assignee: Virongy L.L.C., Manassas, VA (US)

(21) Appl. No.: 18/546,268

(22) PCT Filed: Feb. 14, 2022
(86) PCT No.: PCT/US2022/016309

§ 371 Date: Aug. 12, 2023

Related U.S. Application Data
(60) Provisional application No. 63/149,238, filed on Feb. 13, 2021.

(51) Int. Cl. A61K 38/17; A61K 38/39; A61K 45/06; A61K 48/00

(52) U.S. Cl. CPC A61K 38/1719; A61K 38/39; A61K 45/06; A61K 48/0008

(57) ABSTRACT

The present disclosure relates to combinations and formulations of actin-based peptides with molecules to enhance the bioactivities of actin-based peptides for enhancing their abilities for modulating actin-related cellular bioactivity and cellular susceptibility to viral infection.

### CROSS-REFERENCE TO RELATED APPLICATION

This application claims priority to U.S. Provisional Application No. 63/149,238, filed Feb. 13, 2021, which application is incorporated by reference herein in its entirety.

### FIELD

The present disclosure relates to combinations and formulations of actin-based peptides with molecules to enhance the bioactivities of these actin-based peptides for enhancing their abilities for modulating actin-related cellular bioactivity and cellular susceptibility to viral infection.

### INTRODUCTION

In cells, the actin cytoskeleton provides mechanical support and is a major driving force for cell motility. Actin also participates in many cellular processes such as cell division, surface receptor cycling, vesicle and organelle movement, and signal transduction. In immune cells, actin is also involved in cell adhesion, cell migration, chemotaxis, and T cell activation (Wulfing and Davis, 1998).

Actin-related Cellular Bioactivity: The cytoskeleton of a cell consists of three main filaments: actin filaments (also called microfilaments), intermediate filaments, and microtubules. The cytoskeleton provides a cell with shape, structure, and an anchorage for various organelles. It also provides driving forces for cell migration and intracellular transport of macromolecules. In addition, actin regulates many key functions in cells, including:
The formation of various of actin networks in cells for providing cellular trafficking routes.
Modulation of cell surface receptors. Actin is heavily involved in receptor clustering and cycling.
Cytokinesis. Cell division involves a constracting ring that is composed of actin, myosin, and α-actinin.
Apoptosis. Actin is degraded into two fragments during apoptosis. Stress can induce the formation of stress fibers that is involved in apoptosis. The mitochondrial migration of the actin binding protein cofilin is also involved in triggering cell apopotosis.
Cell adhesion and development. Adhesion of epithelial cells with extracellular matrix or with adjacent cells involves the actin cytoskeleton as well as cadherins and catenins.
Gene expression modulation. Actin's state of polymerization can affect the pattern of gene expression.
Smooth and hear muscle contraction: both actin and myosin are involved in muscle contraction and relaxation, and they make up 90% of muscle protein. The interaction of actin-myosin results in the movement of the two proteins that produces contractions.
Nuclear actin function: actin can also enter the nucleus, and is involved in the architectural integrity of the nucleus, gene transcription, and translocation of the activated chromosome fragment from under membrane to euchromatin.

Actin Filaments: The major cytoskeletal protein of cells is actin, a 43-kd protein that polymerizes to form actin filaments—thin, flexible fibers approximately 7 nm in diameter and up to several micrometers in length. Within the cell, actin filaments are organized into higher-order structures, forming bundles or three-dimensional networks with the properties of semisolid gels. The assembly and disassembly of actin filaments, their crosslinking into bundles and networks, and their association with other cell structures such as the plasma membrane are regulated by a variety of actin-binding proteins which are critical components of the actin cytoskeleton. Actin filaments are particularly abundant beneath the plasma membrane, where they form a network that provides mechanical support, determines cell shape, and allows movement of the cell surface, thereby enabling cells to migrate, engulf particles, and divide. Cooper, G. M., The Cell: A Molecular Approach, 2^{nd} Ed., Sinauer Associates, Sunderland, MA (2000). http://www.ncbi.nlm.nih.gov/books/NBK9908/Actin structure, function and sequences are described by Otterbein, L R, The Crystal Structure of Uncomplexed Actin in the ADP State, Science, Vol. 293, 27 Jul. 2001; Kabsch, W., The Actin Fold, FASEB J. 9, 167-174 (1995); Homes, K. C. et al., Atomic model of the actin filament, Nature, Vol. 347, 6 Sep. 1990; Mattila, P. K. et al., Dynamics of the actin cytoskeleton mediates receptor cross talk: An emerging concept in tuning receptor signaling, J. Cell Biol. Vol. 212 No. 3, 267-280 (2016); Sandiford, S. L. et al. (2015), Cytoplasmic Actin Is an Extracellular Insect, PLoS Pathog 11(2): e1004631. doi:10.1371/journal.ppat.1004631; Hardin, J., Regulating cell-cell junctions from A to Z, J. Cell Biol. Vol. 213 No. 2 151-153 (2016); and Yoder, A. et al., HIV Envelope-CXCR4 Signaling Activates Cofilin to Overcome Cortical Actin Restriction in Resting CD4 T Cells, Cell 134, 782-792, Sep. 5, 2008.

There are three actin isoforms, alpha, beta, and gamma. Alpha actin is mainly found in contractile structures such as those of muscle cells. Beta actin is found at the leading edge of migrating cells to drive cell mobility. Gamma actin is found in the filaments of stress fibers. Actins exist either as a monomeric form (G-actin) or as assembled, double helical, filamentous polymers (filamentous actin, F-actin, or microfilaments).

In cells, the actin cytoskeleton provides mechanical support and is a major driving force for cell motility. Actin also participates in many cellular processes such as cell division, surface receptor cycling, vesicle and organelle movement, and signal transduction. In immune cells, actin is also involved in cell adhesion, cell migration, chemotaxis, and T cell activation (Wulfing and Davis, 1998).

Intracellular actin activity is regulated through actin polymerization and depolymerization, which are regulated by multiple actin modulators in cells. In test tubes, in the absence of other actin regulators, G-actin can automatically polymerize at high salt concentration. Polymerized actin filaments (F-actin) can also dissociate into G-actin. Structurally, polymerized actin filaments have polarity, with (+) and (−) ends (or barbed and pointed ends). When actin polymerization and depolymerization reactions reach a steady-state, G-actin is added to the (+) end and dissociated from the (−) end at equal rates, so the length of the actin filaments remains constant. This process is called actin treadmilling, which resembles a process of actin subunits “walking” from the (+) end to the (−) end (Pollard and Borisy, 2003).

In cells, actin dynamics are tightly regulated to ensure proper and speedy response to stimuli. G-actin is normally associated with cellular regulators such as profilin and thymosin β4. Profilin acts as a nucleotide-exchange factor, which allows the switch from ADP-actin to ATP-actin ready for actin polymerization. Thymosin (34 is an actin-sequestering protein, which serves as a buffering protein for the maintenance of the monomeric actin pool. F-actin is also associated with multiple cellular regulators such as ADF/cofilin and the Apr2/3 complex. ADF/Cofilin is a family of actin-severing proteins which depolymerize F-actin mainly at the (−) end. The Arp2/3 complex is a seven-subunit protein that promotes the growth of new actin filaments. In addition, actin capping proteins such as CapZ and gelsolin can cap the ends of actin filaments, preventing actin polymerization or depolymerization (Pollard and Borisy, 2003).

In cells, actin and microtubule cytoskeleton gives mechanical support and control of cell shape. The actin and microtubule cytoskeleton networks also provide trafficking routes through cytoplasm for intracellular macromolecules and, sometimes, invading bacteria and viruses. In non-muscle cells, actin polymerization and depolymerization provide a major driving force for cell migration through treadmilling, whereas in muscle cells, actin fiber is the scaffold on which myosin proteins generate force to produce muscle contraction. In addition, actin has a wide range of roles, including regulation of cell surface receptors, cell division, cell adhesion and differentiation, T cell/B cell activation, and gene expression.

Actin is also involved in the process of apoptosis, in which cellular stress induces the reorganization of the actin cytoskeleton, giving rise to actin stress fiber. During apoptosis, actin is fragmented into two fragments of 15 kD and 31 kD by the ICE/ced-3 family of proteases, which is one of the major characteristics of apoptotic cells.

The microtubule network also plays important roles in many cellular processes. Microtubules provide means of intracellular transport of organelles and secretory vesicles. They are also involved in the formation of mitotic spindles during cell division (mitosis and meiosis). Drugs targeting microtubules are known to induce apoptosis of cancer cells.

Actin and microtubule dynamics are also important for intracellular bacterial and viral infection of cells. Actin polymerization and depolymerization can modulate receptor dynamics, facilitating the entry of bacteria and viruses. Actin polymerization can produce essential driving forces and scaffolds for bacterial and viral intracellular migration, nucleic acid synthesis and transcription, or virion assembly and budding (Spear et al. 2012; Spear et al. 2013; Taylor et al 2011). The microtubule network is frequently used by intracellular bacteria and viruses for migrations inside and between cells (Kotsakis et al. 2001).

### SUMMARY

In one aspect, a method for the enhancing delivery of a nucleic acid into a cell, comprising introducing into a cell one or more actin-based peptides in an amount sufficient to enhance delivery, wherein the one or more actin-based peptides are selected from peptides having a sequence selected from SEQ ID No:1 to SEQ ID No:845.

In one embodiment, one or more actin-based peptides are introduced in the form of a composition further comprising a second molecule differing from the one or more actin-based peptides.

In one aspect, a method for enhancing cell-cell and cell-extracellular matrix adhesion, comprising treating a cell one or more actin-based peptides in an amount sufficient to enhance cell adhesion, wherein the one or more actin-based peptides are selected from peptides having a sequence selected from SEQ ID No:1 to SEQ ID No:845.

In one embodiment, the one or more actin-based peptides are introduced in the form of a composition further comprising a second molecule differing from the one or more actin-based peptides.

In one embodiment, the molecule is a low-molecular weight molecule or derived polymer, such as polybrene (hexadimethrine bormid), Dextrins, or PEI (polyethylenimine), or any molecule of similar nature for enhancing viral infection and exosome delivery.

In one embodiment, the molecule is a peptide such as cationic peptides including but not limited to Dorsocin, Histatin-5, indolicidin, LL-37, polycathonic amyloid fibrils, the Semen-Derived Enhancer of Viral Infection (SEVI), vectofusin-1, or any peptides of similar nature for enhancing viral infection and exosome delivery.

In one embodiment, the molecule is a peptide, such as cell-permeable peptides including but not limited to small polybasic peptides derived from proteins, for example, the third α-helix fragment of Antennapedia (Antp) homeodomain.

In one embodiment, the molecule is a protein, such as fragments of fibronectin (rFN-CH-296), or any protein of similar nature for enhancing viral infection and exosome delivery.

In one embodiment, the molecule is a liposome such as the phosphatidylserine (PS) liposomes, or any liposome of similar nature for delivery of nucleic acids and proteins into cells.

In one embodiment, the one or more actin-based peptides are incorporated into virion or exosome particles to enhance their ability to infect cells or to enter cells for the delivery of genes and proteins.

In one embodiment, the actin-based peptide has a core sequence selected from NB5 core sequences (SEQ ID Nos: 1 and 15-24).

In one embodiment, the actin-based peptide has a core sequence selected from NB7 core sequences (SEQ ID Nos: 2 and 139-148).

In one embodiment, the actin-based peptide has a core sequence selected from NB9 core sequences (SEQ ID Nos: 3 and 291-299).

In one embodiment, the actin-based peptide has a core sequence selected from NB10 core sequences (SEQ ID Nos: 4 and 471-481).

In one embodiment, the actin-based peptide has a core sequence selected from B11 core sequences (SEQ ID Nos: 5, 13, and 602-723).

In one embodiment, the actin-based peptide has a core sequence selected from B17 core sequences (SEQ ID Nos: 6, 14, and 724-845).

In one embodiment, the actin-based peptide has a core sequence selected from B5 core sequences (SEQ ID Nos: 7 and 25-138); B6 core sequences (SEQ ID Nos: 8 and 272-290); and

B9 core sequences (SEQ ID Nos: 11, 291-299, 340-343, 364-382, and 406-432).

In one embodiment, the actin-based peptide has a core sequence selected from N7 core sequences (SEQ ID Nos: 9, 41, 149-271, 311, and 498); N9 core sequences (SEQ ID Nos: 10, 43, 300-339, 344-363, 383-405, 433-479, and 500); and N10 core sequences (SEQ ID Nos: 12, 44, 314, and 482-601).

In another aspect, a composition comprising a combination one or more actin-based peptides and a second molecule differing from the one or more actin-based peptides, wherein the one or more actin-based peptides are selected from peptides having a sequence selected from SEQ ID No:1 to SEQ ID No:845, and wherein the composition can enhance virus infection of cells or can enhance exosome delivery of genes and proteins into cells.

In one embodiment, the molecule is selected from:
a peptide such as cationic peptides including but not limited to Dorsocin, Histatin-5, indolicidin, LL-37, polycathonic amyloid fibrils, the Semen-Derived Enhancer of Viral Infection (SEVI), vectofusin-1, or any peptides of similar nature for enhancing viral infection and exosome delivery; such as cell-permeable peptides including but not limited to small polybasic peptides derived from proteins, for example, the third α-helix fragment of Antennapedia (Antp) homeodomain; such as fragments of fibronectin (rFN-CH-296), or any protein of similar nature for enhancing viral infection and exosome delivery; or
a liposome such as the phosphatidylserine (PS) liposomes, or any liposome of similar nature for delivery of nucleic acids and proteins into cells.

In one aspect, a composition comprising a combination of an actin-based peptide (SEQ ID No:1 to SEQ ID No:845) with a molecule, wherein said composition can enhance virus infection of cells or can enhance exosome delivery of genes and proteins into cells.

In one embodiment, the molecule is a low-molecular weight molecule or derived polymer, such as polybrene (hexadimethrine bormid), Dextrins, or PEI (polyethylenimine), or any molecule of similar nature for enhancing viral infection and exosome delivery.

In one embodiment, the molecule is a peptide such as cationic peptides including but not limited to Dorsocin, Histatin-5, indolicidin, LL-37, polycathonic amyloid fibrils, the Semen-Derived Enhancer of Viral Infection (SEVI), vectofusin-1, or any peptides of similar nature for enhancing viral infection and exosome delivery.

In one embodiment, the molecule is a peptide, such as cell-permeable peptides including but not limited to small polybasic peptides derived from proteins, for example, the third α-helix fragment of Antennapedia (Antp) homeodomain.

In one embodiment, the molecule is a protein, such as fragments of fibronectin (rFN-CH-296), or any protein of similar nature for enhancing viral infection and exosome delivery.

In one embodiment, the molecule is a liposome such as the phosphatidylserine (PS) liposomes, or any liposome of similar nature for delivery of nucleic acids and proteins into cells.

In another embodiment, the actin-based peptides (SEQ ID No:1 to SEQ ID No:845) or the combination with a molecule can be incorporated into virion or exosome particles to enhance their ability to infect cells or enter cells for the delivery of genes and proteins.

In one embodiment, the peptide N9-A20 enhances delivery of nucleic acids into cells.

In another embodiment, the actin-based peptides (SEQ ID No:1 to SEQ ID No:845) or the combination with a molecule can be conjugated with the structural proteins presented in virion or exosome particles to enhance their ability to infect cells or enter cells for the delivery of genes and proteins.

In another embodiment, the actin-based peptides (SEQ ID No:1 to SEQ ID No:845) or the combination with a molecule can be conjugated with small-molecules, drugs, peptides, or proteins, and can be used for delivery of these small-molecules, drugs, peptides, or proteins into cells, virion particles, or exosomes.

In another embodiment, the small-molecules include but not limited to fluorescence molecules that can be used to monitor viral entry and exosomes entry into target cells.

In another embodiment, the actin-based peptides (SEQ ID No:1 to SEQ ID No:845) can be conjugated with multiple peptides or domains of proteins that can direct bind to nucleic acids (DNA/RNA) and mediate cytoplasmic and nuclear entry, and acts as a nucleic acids and protein transfection agent. In one embodiment, the peptides or domains include but not limited to DNA/RNA binding domains from proteins and nuclear entry signal domains from proteins.

In one embodiment, the actin-based peptides (SEQ ID No:1 to SEQ ID No:845) can be used to enhance surface expression of multiple cellular receptors including, but not limited to CD325 (N-cadherin), CD144 (VE-cadherin), and CD324 (E-cadherin).

In one embodiment, the actin-based peptides (SEQ ID No:1 to SEQ ID No:845) can used to enhance cell-cell and cell-extracellular matrix adhesion. In another embodiment, the peptide N7-20A enhances cell-cell and cell-extracellular matrix adhesion.

In another embodiment, a method for the enhancing delivery of a nucleic acid into a cell, comprising introducing peptide N9-A20 into said cell, in an amount sufficient to enhance delivery.

In another embodiment, a method for enhancing cell-cell and cell-extracellular matrix adhesion, comprising treating a cell with peptide N7-20A, in an amount sufficient to enhance adhesion.

### BRIEF DESCRIPTION OF THE DRAWINGS

FIG. 1: (A) Schematic representation of the actin-core domains discovered from screening. (B) The amino acid sequences of the core domains. (C) The location and AA position of the actin-core domains within the human β-actin protein. (D) β-actin structure, showing subdomains 1 to 4 and the hydrophobic cleft between subdomains 1 and 3 (red circle). This hydrophobic cleft is a common binding pocket of most actin binding proteins (ABPs) such as gelsolin, profilin, and ADF/cofilin as shown.

FIG. 2: Enhancement of lentiviral vector transduction by combination of actin-based peptide and polybrene. Viral transduction of cells was measured at 3 days post infection by flow cytometry. (A) uninfected cells control. (B) Cells were infected with a lenti-GFP vector viral particles. (C) Cells were infected with a lenti-GFP vector viral particles in the presence of an actin-based peptide (B11-20AA) plus polybrane. Transduction efficiency was measured by the percentage of GFP+ cells. PI,_propidium iodide, was added before flow cytometry for cytotoxicity control (FL3). GFP+ cells were counted only when the cells are viable (low PI staining).

FIG. 3: Enhancement of lentiviral vector transduction by combination of actin-based peptide and a cationic peptide. Viral transduction of cells was measured at 3 days post infection by flow cytometry. (A) uninfected cells control. (B) Cells were infected with a lenti-GFP vector viral particles. (C) Cells were infected with a lenti-GFP vector viral particles in the presence of an actin-based peptide (B11-20AA) plus a cationic peptide. Transduction efficiency was measured by the percentage of GFP+ cells. PI,_propidium iodide, was added before flow cytometry for cytotoxicity control (FL3). GFP+ cells were counted only when the cells are viable (low PI staining).

FIG. 4: Enhancement of lentiviral virion infectivity by virion incorporation of actin-based peptide. (A) Uninfected cells control. (B) Lenti-GFP virion particles were used to directly infect target cells. (C) Lenti-GFP virion particles were first incubated with an actin-based peptide (B11-20AA) to allow virion incorporation of the actin-based peptide, and then used to infect cells. An equal amount of virion particles in (B) and (C) were used for infection. Virion infectivity was measured by the percentage of GFP+ cells.

FIG. 5: Example of intracellular delivery of small-molecules by actin-based peptide. (A) untreated cells control. (B) Cells were treated briefly with a FITC-conjugated actin-based peptide (B11-20AA). The intracellular delivery of the FITC-actin-based peptide was quantified by flow cytometry. As shown in (B), the FITC-actin-based peptide can be delivered into 100% of cells.

FIG. 6: Example of combination of actin-based peptide with liposome to deliver nucleic acids into cells. HEK293T cells were transfected with a GFP-expression vector DNA (Lenti-GFP) in complex with liposome (DNA+Lipo) or with liposome and an actin-based peptide (N9-A20) (DNA+Lipo+N9). Following transfection, cells were quantified for GFP expression at 48 hours post transfection by flow cytometry.

FIG. 7: Example of actin-based peptide to enhance surface expression of multiple cellular receptors including CD325 (N-cadherin), CD144 (VE-cadherin), and CD324 (E-cadherin), which are surface molecule for cell-cell and cell-extracellular matrix binding and adhesion. (A) Human H9 lymphocyte (derivative of HutT 78 cell, from ATCC) were not treated or treated with an actin-based peptide (N7-20AA) for 40 minutes. Cells were stained on ice with PerCy-Cy5.5-labeled mouse anti-human CD325 antibody. Surface expression of CD325 before and after N7-20AA was quantified by flow cytometer. Shown are up-regulation of surface expression of CD325 after N7-20A treatment. (B) Human H9 lymphocyte were not treated or treated with an actin-based peptide (N7-20AA) for 40 minutes. Cells were stained on ice with FITC-labeled mouse anti-human CD144 antibody. Surface expression of CD144 before and after N7-20AA was quantified by flow cytometer. Shown are up-regulation of surface expression of CD144 after N7-20A treatment. (C) Human H9 lymphocyte were not treated or treated with an actin-based peptide (N7-20AA) for 40 minutes. Cells were stained on ice with PE-labeled mouse anti-human CD324 antibody. Surface expression of CD324 before and after N7-20A was quantified by flow cytometer. Shown are up-regulation of surface expression of CD324 after N7-20AA treatment.

### DETAILED DESCRIPTION

Actin is involved in: 1) supporting cell shape and cell growth; 2) driving cells to migrate; 3) cell signal transduction; 4) cell surface receptor cycling between the surface and the cytoplasm; 5) cell-cell junction and cell adhesion; 6) muscle cell contraction; 7) in the cardiovascular system, actin activity affects heart beat and blood vessel elasticity and capacitance; 8) the process of viral and bacterial infection. Actin can function as a barrier to block viral infection, while viruses and bacteria may also use actin polymerization to promote inter- and intracellular migration; 9) the intracellular delivery of chemical compounds (e.g. small-molecule drugs) and biological substances (e.g. DNA, peptides, and proteins et al). The cortical actin in a cell structure can also block the intracellular delivery of compounds and substances, affecting the delivery efficiency; 10) the process of cell growth, differentiation and apoptosis.

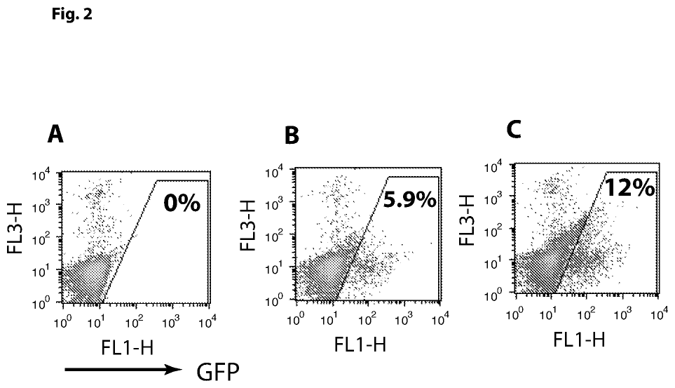

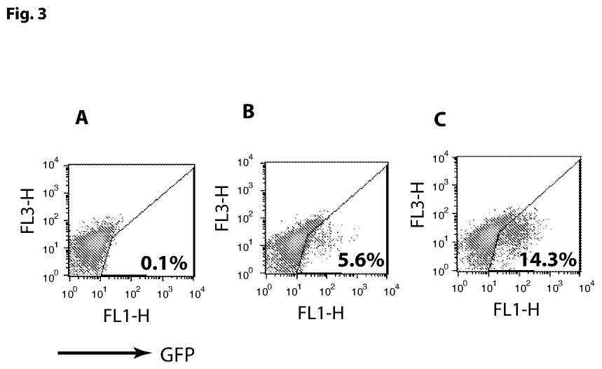

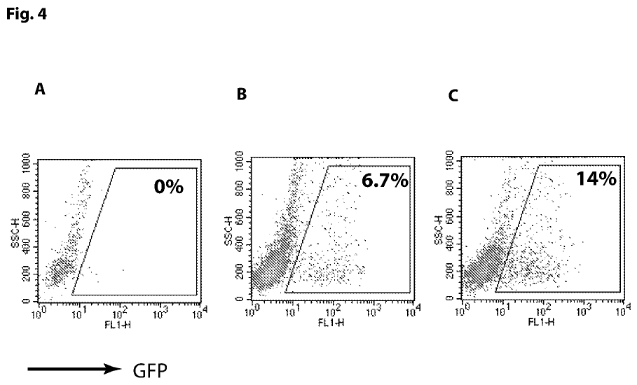

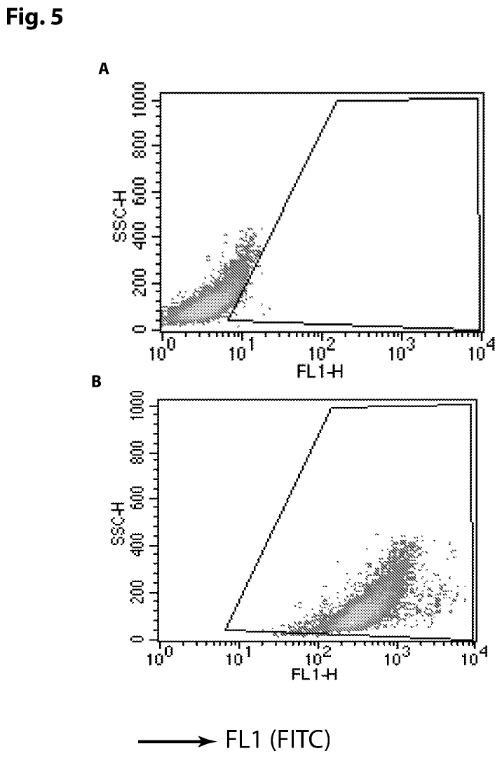

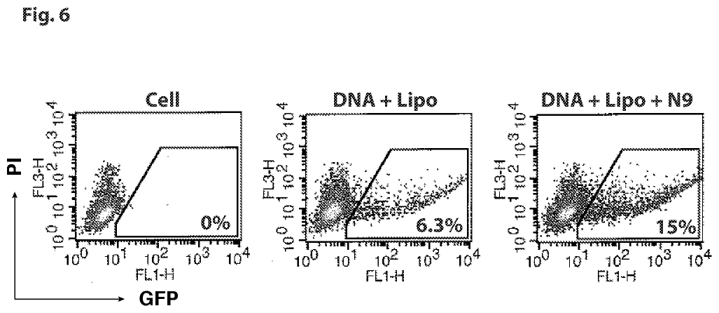

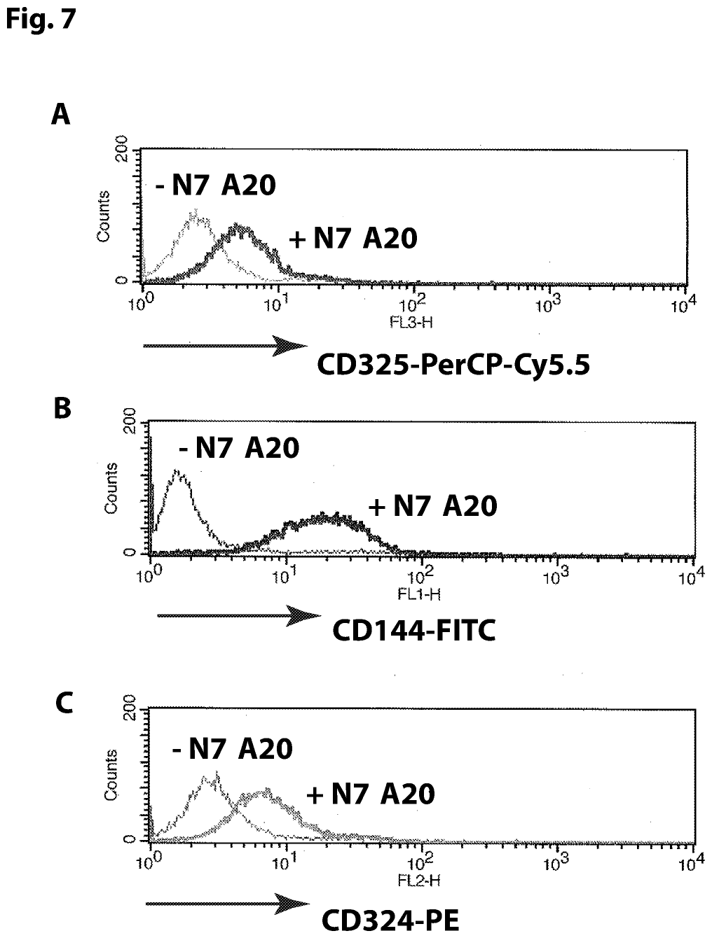

The crystal structure of G-actin coupled with DNase I was solved in 1990 by Kabsch and colleagues [5]. The crystal structure of uncomplexed actin was also solved by Otterbein and coauthors [2]. The near-atomic resolution structure of F-actin was deduced by Holmes and colleagues using a combination of the crystal structure of G-actin and X-ray fiber diffraction data [3]. Currently, there are also over 80 structures of actin in complex with various actin binding proteins such as gelsolin, cofilin, and profilin [, #3346]. Structurally, actin is organized into two related domains, which can be further subdivided into 4 subdomains numbered 1 to 4 (FIG. 2D).

β-actin structure, showing subdomains 1 to 4 and the hydrophobic cleft between subdomains 1 and 3 (red circle). This hydrophobic cleft is a common binding pocket of most actin binding proteins (ABPs) such as gelsolin, profilin, and ADF/cofilin as shown. Two diametrically opposed clefts separate the two large domains of actin. The larger cleft, between subdomains 2 and 4, constitutes the nucleotide-binding site, whereas the smaller cleft, between subdomains 1 and 3, mediates the interactions of actin with most actin binding proteins (ABPs). ABPs are extremely diverse, both structurally and functionally, but for some reason related to the filament structure, most ABPs seem to share a common binding area on the actin surface, which is the hydrophobic pocket between actin subdomains 1 and 3. Both gelsolin and cofilin bind to this cleft, and profilin also interacts with the back of this cleft. It is possible that our peptides interface with the binding of some ABPs, thereby disturbing actin dynamics, and indirectly the dynamics of surface receptors.

As contemplated herein, a composition can be delivered into a cell in any form by any effective route, including but not limited to oral, parenteral, enteral, intraperitoneal, topical, transdermal (e.g., using any standard patch), ophthalmic, nasally, local, non-oral, such as aerosol, spray, inhalation, percutaneous (epidermal), subcutaneous, intravenous, intramuscular, buccal, sublingual, rectal, vaginal, intra-arterial, mucosal, and intrathecal. A composition can be administered alone, or in combination with any ingredient(s), active or inactive.

Without being bound by theory, it is thought that the actin-based peptides of the invention are likely to act through three different mechanisms: 1) the peptides disturb actin polymerization and actin depolymerization at the plus and minus ends. Thus, disturbing the process of actin treadmilling; 2) these peptides may compete directly with actin binding proteins such as cofilin, Arp2/3, thus, affecting actin dynamics; 3) these peptides may compete with actin binding adaptor proteins or actin binding motifs present on the intracellular domains of receptors.

Actin-peptides can inhibit or enhance viral infection processes at multiple steps, such as: 1) enhance or inhibit of viral entry; 2) enhance or inhibit viral intracellular migration, 3) enhance or inhibit viral nuclear migration, 4) enhance or inhibit viral assembly and budding; 5) enhance or inhibit viral cell-cell transmission.

All technical terms in this description are commonly used in biochemistry, molecular biology and immunology, respectively, and can be understood by those skilled in the field of this invention. Those technical terms can be found in: MOLECULAR CLONING: A LABORATORY MANUAL, 3rd ed., vol. 1-3, ed. Sambrook and Russel, Cold Spring Harbor Laboratory Press, Cold Spring Harbor, N.Y., 2001; CURRENT PROTOCOLS IN MOLECULAR BIOLOGY, ed. Ausubel et al., Greene Publishing Associates and Wiley Interscience, New York, 1988 (with periodic updates); SHORT PROTOCOLS IN MOLECULAR BIOLOGY: A COMPENDIUM OF METHODS FROM CURRENT PROTOCOLS IN MOLECULAR BIOLOGY, 5.sup.th ed., vol. 1-2, ed. Ausubel et al., John Wiley & Sons, Inc., 2002; GENOME ANALYSIS: A LABORATORY MANUAL, vol. 1-2, ed. Green et al., Cold Spring Harbor Laboratory Press, Cold Spring Harbor, N.Y., 1997; CELLULAR AND MOLECULAR IMMUNOLOGY, 4.sup.th ed. Abbas et al., WB Saunders, 1994.

Interaction of viruses with actin cytoskeleton: The actin cytoskeleton exists as a polarized array of filaments, termed F-actin, in dynamic equilibrium with a globular actin, or G-actin, pool. Polymerization of the F-actin filament occurs predominately at the barbed end (also known as the plus end), while depolymerization occurs predominantly at the pointed end (also known as the minus end). The precise spatiotemporal regulation of actin polymerization, mediated by actin-binding proteins (ABPs) and their upstream regulators, coordinates the force-generating and scaffolding properties of actin. These, in turn, regulate complex cellular processes including chemotaxis, cell adhesion, cytokinesis, the formation of cellular processes (microvilli, filopodia, lamellipodia, invadopodia, etc.), organelle movement, endocytosis, and the assembly of macromolecular domains during signal transduction and receptor internalization.

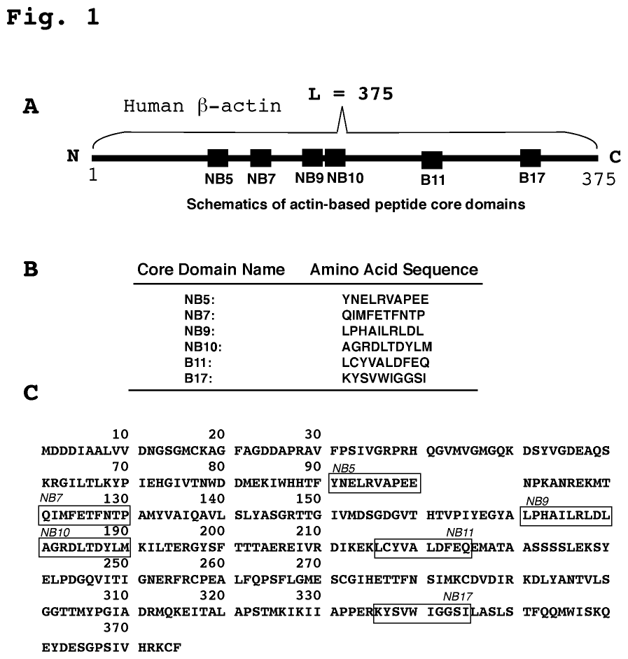

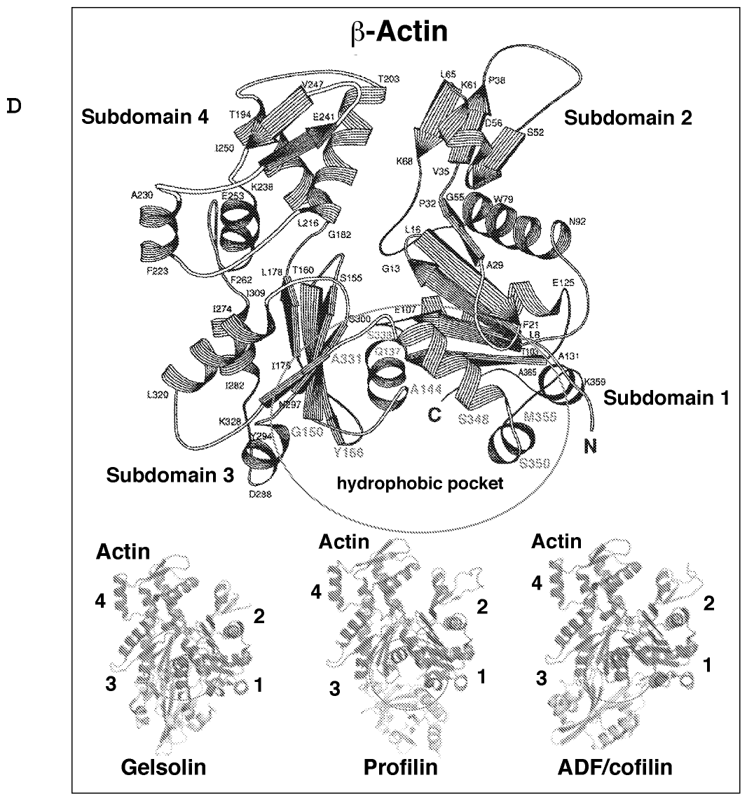

These primary functions of the actin cytoskeleton are themselves regulated by the actin-modulating ABPs, which exhibit various activities towards F-actin, including bundling and crosslinking (Fimbrin, Fascin, Filamin A, etc.), membrane and receptor association (ERM proteins, among others), capping (CapZ, tropomodulin, etc.), anti-capping (Ena/VASP proteins), nucleating and branching (the Arp2/3 complex), nucleation and polymerizing (Spire1/2 and formins), actin-associated motor activity (Myosins), and severing functions (cofilin, gelsolin, etc.). Directly upstream of these factors and their associated activities are the Rho-family monomeric GTPases, which are activated by nucleotide exchange of bound GDP to GTP. The Rho GTPases, which include 20 members, are divided into canonical signaling motifs that mediate particular cellular processes (FIG. 1). Among these are the Rac1-PAK-LIMK-Cofilin pathway, which terminates with LIMK phosphorylating and inactivating cofilin; the Rac1-WAVE-Arp2/3 pathway, which promotes actin nucleation and branching, and lamellipodium generation; the Cdc42-WASP-Arp2/3 and Cdc42-mDia pathways, which activate Arp2/3 and the formin, mDia, respectively, and promote actin nucleation, branching, polymerization, and the formation of filopodia; and the RhoA-ROCK-LIMK-Cofilin and RhoA-ROCK-MLC pathways, which are partially responsible for the assembly of the contractile uropod, stress fibers, and focal adhesions.

It is these force-generating and scaffolding properties exhibited by the actin cytoskeleton and its effectors that frequently become targets of necessity for viral replication, which also require these activities. As discussed below, functions such as Arp2/3 nucleation and branching and formin-mediated actin polymerization are co-opted to produce force for viral motility. Similarly, the normal mechanisms of actin rearrangement, such as cofilin activation and inactivation cycling, are dysregulated to create favorable microcompartments for genome replication and nuclear localization. As such, each stage in the viral life cycle, from entry to egress and dissemination, will be considered in turn, explicating the various motifs of viral subversion of normal cellular actin signaling functions.

As a fundamental component of the host cellular cytoskeleton, actin is routinely engaged by infecting bacterial and viruses. Bacteria and Viruses from diverse groups, and infecting diverse hosts, have convergently evolved an array of mechanisms for manipulating the actin cytoskeleton for efficacious infection. The actin cytoskeleton is critical for viral infection at many stages of the life cycle, including binding, cell entry, nuclear localization, genomic transcription and reverse transcription, assembly, and egress/dissemination. Specifically, viruses subvert the force-generating and macromolecular scaffolding properties of the actin cytoskeleton to propel viral surfing, internalization, and migration within the cell. Additionally, viruses utilize the actin cytoskeleton to support and organize assembly sites, and eject budding virions for cell-to-cell transmission.

Actin and Viral Entry: After engagement of the extracellular matrix (ECM) and the viral receptor, viruses must migrate to sites favorable for their particular mode of entry, whether it is direct plasma membrane fusion, or routes as divergent as macropinocytosis, phagocytosis, and the various forms of clathrin-mediated and clathrin-independent endocytosis. During this process, viruses frequently encounter actin-based processes, such as filopodia and microvilli, and utilize these structures to efficiently migrate to the cell membrane for entry.

Among the most common forms of this process is virus surfing, in which engagement of the receptor or ECM promotes movement of the attached virion towards the cell body in an actin-dependent mechanism. For instance, Lehmann et al. exhibited Murine leukemia virus (MLV), Avian leukosis virus (ALV), and Human immunodeficiency virus (HIV) surfing along filopodia in HEK293T cells transfected with their respective receptors (Lehmann M J, et al., 2005). MLV particles, in particular, migrated to the base of the filopodia and fused with the membrane, indicating that this was the active, and perhaps predominant, entry location (Lehmann M J, et al., 2005). Furthermore, this viral surfing was energy-dependent, being inhibited by sodium azide, and was dependent on functional myosin II and F-actin: treatment of cells with the myosin II inhibitor, blebbistatin, or cytochalasin D, which blocks F-actin barbed ends from polymerization, eliminated virus surfing and promoted random motility at the plasma membrane (Lehmann M J, et al., 2005). These treatments additionally inhibited MLV and ALV viral infection in rat XC cells, indicating that the viral surfing route contributed to productive infection (Lehmann M J, et al., 2005). The model developed around this process is centered around actin retrograde flow in which myosin II generates forces that pull the filopodia-associated actin filaments towards the cell body, which is commensurate with viral receptor-actin association and filopodial tip actin polymerization. The net result is that the virus-receptor complex is processively pushed and dragged towards the cell body where entry occurs.

Viral surfing on filopodia has also been described for Herpes simplex virus type 1 (HSV-1) (Clement C, et al., 2006; Dixit R, et al., 2008), vaccinia virus (Mercer J, et al., 2008), Human papilloma virus 16 (HPV-16) (Schelhaas M, et al., 2008), Hepatitis C virus (HCV) (Coller K E, et al., 2009), and HIV-1 (Lehmann M J, et al., 2005), indicating that utilization of actin retrograde flow in cellular processes, particularly filopodia, may be a general mechanism for extracellular viral migration towards the cell body. To some extent, the intracellular migration of viruses is a virus-dependent active process, requiring signal transduction from or clustering of the viral receptor. For instance, HSV-1 not only migrated upon filopodia, but dramatically induced their formation in P19 neuron-like cells (Dixit R, et al., 2008). This indicates that HSV-1 engagement of the viral receptor specifically introduces signaling events to mediate filopodia formation, implicating that Rho and PI3K-dependent pathways are critical for mediating cytoskeletal structures essential for viral infection (Clement C, et al., 2006; Zheng K, et al., 2014b).

As indicated above for HSV-1, another common theme preceding cell entry is that virus-receptor engagements can actively signal to the actin cytoskeleton. This receptor-mediated signaling can dramatically affect entry events, promoting receptor clustering, receptor endocytosis, and fusion complex stabilization. Additionally, these signal transduction events often have important impacts on subsequent events in the viral life cycle. For instance, binding of HIV-1 to the chemokine coreceptor CXCR4/CCR5 triggers the activation of Rac1-PAK1/2-LIMK-cofilin (Yoder A, et al., 2008; Vorster P J, et al., 2011). This signaling pathway regulates actin dynamics and the surface cycling of CXCR4, facilitating viral entry and post entry DNA synthesis and nuclear migration (Vorster P J, et al., 2011). Clustering of HIV-1 viral receptor and coreceptor (CD4 and either CCR5 or CXCR4) has also been suggested to be dependent on viral receptor signaling: specifically, engagement of both CD4 and CXCR4 activates Filamin A (Jiménez-Baranda S, et al., 2007), which promotes receptor motility and clustering. The ERM protein, Moesin, was also found to play a similar role in HIV-1 receptor clustering, in which the membrane receptor-actin crosslinking activity and receptor clustering occur after a viral receptor-mediated Moesin phosphorylation event (Barrero-Villar M, et al., 2009). It is probable that many other viruses utilize the actin cytoskeleton to induce receptor clustering and promote viral entry.

After engagement with the viral receptor, viral entry occurs either at the plasma membrane directly, or during/after internalization by one of the endocytic routes. Many, and perhaps all, of these routes depend on actin to one extent or another, particularly in primary, differentiated cells. For instance, macropinocytosis, which involves the Rac1-PAK-LIMK-Cofilin and, possibly, Rac1-WAVE-Arp2/3 pathways, has been shown to be required for HIV-1 entry into macrophages (Carter G C, et al., 2011). Macropinocytosis is also required for the entry of African swine fever virus (ASFV) (Sanchez E G, et al., 2012) and HPV-16 (Schelhaas M, et al., 2012). It also serves as an alternate entry route for Influenza A virus (IAV) (de Vries E, et al., 2011). An interesting twist on this is found in vaccinia virus entry, where viral envelope-associated phosphatidylserine induces an apoptotic body-mimicking signaling pathway, inducing a Rac1, PAK, tyrosine kinase, and actin-dependent macropinocytic entry route. Although, as reviewed by Moss (Moss B, 2012), the poxvirus entry fusion complex—composed of 11-12 glycosylated proteins—is abnormally complex, and other entry routes exist. Huttunem et al. (2014) also implicate a Rac1-dependent entry mechanism, involving neutral multivesicular bodies, in Coxsackievirus A9 entry (Huttunen M, et al., 2014). Similarly, HSV-1 has been shown to utilize a RhoA, tyrosine kinase, and actin-dependent phagocytosis-like route for entry (Clement C, et al., 2006; Zheng K, et al., 2014b). Poliovirus also utilizes an actin and tyrosine-kinase dependent route for entry, wherein genomic RNA release occurs in vesicles or membrane invaginations within 100-200 nm of the plasma membrane (Brandenburg B, et al., 2007). These examples, including many viruses from divergent groups, illuminate the need to co-opt the force generating and scaffolding functions of the cortical actin cytoskeleton, and the signaling pathways that modulate these functions, during viral entry.

Actin and nuclear migration: After accessing the cortisol, most viruses must traverse the gap between the entry site and the nucleus for expression, genome replication, and, for some viruses, assembly. It is to this end that viruses utilize cellular force-generating apparatuses, which, along with dynein and kinesin for microtubule-based transport, include the motor and polymerizing proteins for the actin cytoskeleton; specifically, spire, formins, Arp2/3, and myosins. For instance, HIV-1 migrates to the nucleus utilizing two downstream components of Rac signaling, the Rac1-PAK1/2-LIMK-Cofiln pathway (Yoder A, et al., 2008; Vorster P J, et al., 2011), and the Rac1-WAVE2-Arp2/3 signaling pathway (Spear M, et al., 2014). The initial stimulus for Rac activation was also shown to require coreceptor— CCR5 or CXCR4—engagement and signal transduction (Yoder A, et al., 2008; Vorster P J, et al., 2011; Spear M, et al., 2014). Interestingly, from a potential therapeutic standpoint, treatment of CD4 T lymphocytes with the Arp2/3 inhibitor, CK-548 (Nolen B J, et al., 2009), dramatically reduced HIV-1 replication at dosages that did not affect CD4 T cell activation or induce cytotoxicity (Spear M, et al., 2014). For viruses with larger genomes, more elaborate mechanisms for mediating nuclear localization exist. This is the case with baculovirus, where the p78/83 capsid encodes a WASP/WAVE homology domain, the WASP homology-Cofilin homology/Connector-Acidic domain (WCA) (Machesky L M, et al., 2001). This domain is normally found in the Nuclear Promoting Factors (NPFs) of the WASP/WAVE family of proteins, whereupon NPF activation leads to WCA exposure and activation of the Arp2/3 complex for actin polymerization (Higgs H N, et al., 1999, 2001; Pollard T D, et al., 2003). However, in the presence of baculovirus p78/83, the viral WCA domain activates Arp2/3 at the viral core surface, generating forces and an actin comet tail that propel the core to the nucleus for expression and replication (Goley E D, et al., 2006; Ohkawa T, et al., 2010). As will be discussed later, nuclear translocation of the Arp2/3 complex, nuclear F-actin polymerization, and the resultant perturbation of nuclear structure also play a critical role during viral assembly and egress (Ohkawa T, et al., 1999; Goley E D, et al., 2006). HSV-1 has also been shown to utilize actin for intracellular motility and nuclear localization in a process involving the cofilin phosphatase and activator, slingshot, and calcium-regulated protease, calpain (Zheng K, et al., 2014a). The siRNA knockdown of slingshot and calpain reduced viral infection and led to a perinuclear localization of the viral cores, whereas wild-type virus localized to the nucleus efficaciously. In a somewhat stunning display of the requirements for specificity in actin signaling at distinct stages of viral replication, at later time points (8 hours post-infection and beyond), HSV-1 promotes ubiquitin/proteasome-dependent degradation of slingshot, leading to cofilin activation and promoting viral replication (Xiang Y, et al., 2014).

Actin in assembly: Once localized to the nucleus, or cytoplasm for large dsDNA viruses, viruses must concentrate, organize, and assemble their structural proteins, early-acting accessory and catalytic proteins, and genomic nucleic acids prior to egress of the viral particle. Needless to say, this requirement for colocalization of viral proteins, viral nucleic acids, and cellular factors requires scaffolding functions, and filamentous actin is often subverted for this purpose. This was shown for baculovirus: specifically, that mutation of the baculoviral CA domain dramatically abrogated nuclear F-actin accumulation and viral replication; furthermore, that viral progeny produced by one such mutant were distorted, either lacking an envelope altogether or having capsids that did not align with their envelope (Goley E D, et al., 2006).

In a similar fashion, Potato virus X (PVX), radically rearranges the ER and Golgi membranes and host actin into enormous assembly sites called the X-body (Tilsner J, et al., 2012). This process is mediated by the triple-gene block proteins (TGB1, 2, and 3), which are involved in cell-to-cell transfer through plasmodesmata, with PVX TGB1 being able to produce X-like-bodies when expressed alone (Tilsner J, et al., 2012). Although the role of actin in the X-body, and its relation to assembly was not specifically addressed, it has been shown that actin is required for intercellular transfer of PVX, in addition to Tobacco mosaic virus (TMV) and Tomato bushy stunt virus (TBSV), through plasmodesmata (Harries P A, et al., 2009). Thus, it is tempting to speculate that, even for certain plant viruses, actin may contribute to both assembly and cell-to-cell transfer.

Assembly of Influenza A virus (IAV) filamentous particles has also been shown to partially require an intact cortical actin cytoskeleton, as disruption with cytochalasin D (CCD) dramatically abrogated the titers of the filamentous, but not spherical, virions (Roberts P C, et al., 1998). This was later corroborated by Simpson-Holley et al. (2002), showing that treatment of MDCK cells with CCD, jasplakinolide (Jas), or latrunculin A (LatA) dramatically redistributed plasma membrane hemagglutinin (HA), lipid rafts, and actin into annular structures; there was also a commensurate loss of cell surface HA-containing filamentous projections and filamentous virus production (Simpson-Holley M, et al., 2002a). The authors attributed the loss of filamentous viral assembly not to actin playing a specific role in assembly, per se, but rather mobilizing and recruiting lipid rafts to the assembly sites of filamentous particles, which, given their size, require a much larger pool of lipids than their spherical particle counterparts. As such, with lipid rafts, actin, and HA reorganized into small annuli, only spherical particle production occurs owing to the lack of sufficient lipid raft material for the larger filamentous particles (Simpson-Holley M, et al., 2002b).

Measles virus (MeV) has also been shown to utilize actin during assembly. For instance, disruption of the actin cytoskeleton rapidly reduced MeV particle release from the plasma membrane (Stallcup K C, et al., 1983). Furthermore, actin has been observed to colocalize with budding MeV, with meromyosin-labeled actin barbed ends protruding into the nascent virions in close juxtaposition with the viral nucleocapsid (Bohn W, et al., 1986). Additionally, the viral Matrix protein (M) was found to associate with F-actin, and this was shown to reduce the interaction with viral hemagglutinin (H) protein, as CCD treatment dramatically reduced actin co-immunoprecipitating with M, while enhancing H co-immunoprecipitation (Wakimoto H, et al., 2013). This study also indicated that there is a tradeoff between M-actin affinity and viral infectivity, wherein a higher affinity between M and H increases the amount of H, and hence the infectivity, of cell-free virus at the expense of cell-cell fusion. Furthermore, mutations in the M protein affecting M-actin affinity may arise in cell types where cell-cell fusion is more productive than free viral infection (Wakimoto H, et al., 2013). In another study, Dietzel et al. (2013) exhibited that, while CCD treatment enhanced the rate of cell-cell fusion, it also reduces co-transport of M-RNP to the cell surface (Dietzel E, et al., 2013). Additionally, Jas treatment blocked free-virus production, while not affecting M-RNP co-transport, indicating that a dynamic actin cytoskeleton is required for cell-free virus maturation and release (Dietzel E, et al., 2013).

ROCK, LIMK, and cofilin have also been implicated in HIV-1 and Mason-Pfizer monkey virus assembly, release, and cell-cell transmission (Wen X, et al., 2014). Knockdown of LIMK did not inhibit HIV-1 maturation, yet, fully enveloped particles remained associated with the plasma membrane as mature virion aggregates (Wen X, et al., 2014). LIMK was also shown to be recruited to assembly sites (Wen X, et al., 2014). Although the exact mechanism for ROCK-LIMK-Cofilin contribution to virion release remains incompletely resolved, the authors suggested a factor retains the viral particles when normal cytoskeletal dynamics are disrupted (Wen X, et al., 2014).

Actin and cell-cell transmission: Cell-to-cell transmission of animal viruses is a highly efficacious route of dissemination that minimizes interactions with the innate and adaptive immune system. Additionally, this process can render certain antivirals less effective, giving the issue increasing prominence in therapeutic research (Agosto L M, et al., 2014). It constitutes the infection of neighboring cells by an infected cell, typically mediated by the fusogenic surface protein or envelope glycoprotein. Perhaps the best-studied example of this is Vaccinia virus cell-cell transmission (reviewed in (Welch M D, et al., 2013)). In 1976, Vaccinia virions were found to project from CCB-sensitive microvilli-like structures (Stokes G V, 1976); later, it was discovered that these virus-associated projections contained, in addition to actin, α-actinin (Hiller G, et al., 1979), fimbrin, and filamin (Hiller G, et al., 1981; Krempien U, et al., 1981). Additionally, Intracellular Enveloped Virions (IEVs) were shown to induce CCD-sensitive actin comet tails, reminiscent of Listeria, Shigella and Rickettsia infections (Cudmore S, et al., 1995). The actin comet-inducing factor, as identified by phenotypic characterization of deletion mutant viruses, was viral A36, a type 1b membrane protein (Röttger S, et al., 1999) that becomes tyrosine phosphorylated (Frischknecht F, et al., 1999a) at Y112 and Y132 (Frischknecht F, et al., 1999b). Y112 phosphorylation recruits the adaptor protein, Nck, via its SH2 domain (Frischknecht F, et al., 1999b), while Y132 was shown to recruit another adaptor protein, Grb2 (Scaplehorn N, et al., 2002). These proteins, in turn, help to recruit the N-WASP NPF complex (Frischknecht F, et al., 1999b; Moreau V, et al., 2000), which mediates Arp2/3 activation via its WCA domain. The result of this signaling cascade is the production of actin comet tails. When IEVs fuse with the plasma membrane, becoming Cell-associated Enveloped Virions (CEVs), viral A36 remains in the plasma membrane below the virion, promoting the production of the long, microvilli-like structures identified in 1976, and promoting viral dissemination (Welch M D, et al., 2013). Other factors that promote and regulate vaccinia virus motility are continually being discovered, including the formin, FHOD1, its upstream regulator, Rac1 (Alvarez D E, et al., 2013), and casein kinase 2 (Alvarez D E, et al., 2012).

HIV has also been shown to utilize an actin-dependent (Jolly C, et al., 2004, 2007) mode of transmission between T cells, in which viral Env glycoprotein on the donor cell organizes a polarized Virological Synapse (VS) that mediates highly efficient cell-cell transmission. This process may be up to 18,000 times more efficacious that free viral infection (Chen P, et al., 2007). Morphologically, the VS and associated signaling complex, which contains the viral receptor, CD4, and viral coreceptor, CXCR4 or CCR5, resembles the Supramolecular Activation Complex (SMAC) observed in T cell activation (Vasiliver-Shamis G, et al., 2008), including the incorporation of the T cell Receptor (TCR) (Vasiliver-Shamis G, et al., 2009). Although the derived signals are insufficient to promote T cell activation, they do create an actin-depleted zone, which may facilitate viral infection at its earliest stages (Vasiliver-Shamis G, et al., 2009). Additionally, HIV has been shown to transfer between T cells using cellular nanotube processes, which may mimic aspects of the VS (Sowinski S, et al., 2008). An interesting twist on these forms of cell-to-cell transmission can be found in infected dendritic cells (DCs), wherein viral filopodia, containing immature virions at their tips, were observed (Aggarwal A, et al., 2012). These viral filopodia were found to be quite dynamic, allowing up to 800 CD4 T cell contacts per hour (Aggarwal A, et al., 2012). Additionally, these viral filopodia partially required the formin, Diaphanous-2 (Diaph2), and the viral Nef accessory protein (Aggarwal A, et al., 2012).

Other roles of actin: In addition to the more canonical modes of viral cooption of the actin cytoskeleton thus described, there is accumulating evidence that cytosolic and nuclear actin may play important roles in viral transcription, translation, and genome replication though somewhat unique modalities.

For instance, in HIV-1 infection, after entry into the host cell, the viral core is deposited on a dense meshwork of cortical actin that undergoes dynamics related to signal transduction mediated by the viral Env-CD4 and Env-CXCR4/CCR5 interactions (Jiménez-Baranda S, et al., 2007; Harmon B, et al., 2008, 2010; Yoder A, et al., 2008; Barrero-Villar M, et al., 2009; Vorster P J, et al., 2011; Spear M, et al., 2014). It is in this submembranous, dynamic actin cortex that the Reverse Transcriptase Complex (RTC) must convert the ssRNA genome into dsDNA. The first indication that the RTC may utilize the actin cytoskeleton came in 1995, when Hottiger et al. exhibited an interaction between beta-actin and the large subunit of Reverse Transcriptase (RT), or the Pol polyprotein precursor (Hottiger M, et al., 1995). Later, Bukrinskaya et al. (1998) corroborated these findings, showing that the RTC does indeed associate with the actin cytoskeleton in infected cells, and that pretreating cells with CCD, but not treatment 2 hours post-infection, reduced the accumulation of early and late reverse transcription products, with a more severe phenotype for late products (Bukrinskaya A, et al., 1998). These results indicated that actin disruption may directly impact the function of RT (Bukrinskaya A, et al., 1998). As was corroborated by later studies, CCD pretreatment reduced the nuclear accumulation of late RT products by 25-fold (Bukrinskaya A, et al., 1998). This process is presumably related to both the reliance on F-actin for both reverse transcription and nuclear migration of the Pre-Integration Complex (PIC) (Yoder A, et al., 2008; Cameron P U, et al., 2010; Vorster P J, et al., 2011; Spear M, et al., 2014). Although, the exact mechanism by which F-actin contributes to RTC activity remain unresolved. Nuclear actin bundles have also been implicated in late gene (gag) mRNA nuclear export and expression (Kimura T, et al., 2000): specifically, treatment with LatA, which disrupted viral Rev-RNP-induced nuclear actin bundles, caused retention of gag mRNA in the nucleus, decreasing cytosolic gag mRNA and, presumably, reducing Gag and Gag-Pol protein expression (Kimura T, et al., 2000).

Rac1 has also been shown to be important for Influenza A virus (IAV) polymerase complex activity (Dierkes R, et al., 2014). Treatment of cells with a Rac1 inhibitor, NSC23766 (Gao Y, et al., 2004), reduced viral replication in A549 cells with an IC₅₀ of 22 μM (Dierkes R, et al., 2014). Furthermore, knockdown of TIAM1, the Rac1-activating GEF, or Rac1 itself also reduced viral replication (Dierkes R, et al., 2014). Additionally, NSC23766 reduced viral protein expression, which was later linked to inhibition of the viral polymerase complex (Dierkes R, et al., 2014). Fascinatingly, mice infected with IAV and treated with NSC23766 showed reduce titer of IAV in lung tissue, increased body weights, and a higher survivability than solvent-treated control mice (Dierkes R, et al., 2014). Although there was no specific study of whether NSC23766-mediated inhibition of the viral polymerase complex required actin, it is likely that actin played some role.

Actin activities are also required for viral infection of target cells. As demonstrated in previous studies (e. g. Summarized by Spear et al., 2012; Spear et al., 2013; Spear and Wu, 2014), actin activities are involved in viral entry, and post entry intracellular migration. Actin-derived peptides can enhance actin activities and facilitate viral entry and intracellular migration. In general, the actin-derived peptides can be modified in order to penetrate cell or virion membrane, e.g., by addition of an N-terminal polyarginine segment. Relevant reviews of cell-penetrating peptides are: C. Bechara et al., Cell-penetrating peptides: 20 years later, where do we stand?, FEBS Letters 587 (2013) 1693-1702; B. Gupta, et al., Intracellular delivery of large molecules and small particles by cell-penetrating proteins and peptides, Advanced Drug Delivery Reviews 57 (2005) 637-651; M. Lindgren, et al., cell-penetrating peptides, TIPS—March 2000 (Vol. 21) 99-103; M. Kristensen, et al., Applications and Challenges for Use of Cell-Penetrating Peptides as Delivery Vectors for Peptide and Protein Cargos, Int. J. Mol. Sci. 2016, 17, 185; and F. Milletti, Cell-penetrating peptides: classes, origin, and current landscape, Drug Discovery Today, Vol. 17, No 15-16, August 2012.

Some virions such as lentiviral virion also contain membrane that is derived from cells. The modified actin-derived peptides that can also be delivered and incorporated into virion particles to become a part of the virion structure. Such virion particles would have higher infectivity.

In addition to enhancing viral infection by actin-based peptides, other molecules, such as polybrene (hexadimethrine bormid) and cationic peptides (e.g. Dorsocin, Histatin-5, polycathonic amyloid fibrils). Such cationic polymers can non-specifically enhance viral absorption on cell surfaces by altering the surface charges of virions and target cells (For example, as described by Fenard et al., 2013). In addition, liposomes such as the phosphatidylserine (PS) liposomes can also enhance viral infection by promoting viral fusion with the cell membrane as described by Coil et al. (Coil and Miller, 2005). Cell-permeable peptides such as small polybasic peptides derived from the transduction domains of certain proteins, such as the third α-helix of the Antennapedia (Antp) homeodomain, can cross the cell membrane through a receptor-independent mechanism. These cell-permeable molecules have been used as ‘Trojan horses’ to introduce biologically active cargo molecules such as DNA, peptides or proteins into cells (Gratton et al., 2003). All of these molecules use a different mechanism to enhance viral infection, as to that of actin-based peptides that enhance viral infection by facilitating viral penetration of the cortical actin.

Exemplary Sequences

The following sequences are essentially derived from the core domains of the actin-based bioactive peptides: NB5, NB7, NB9, NB10, B11, B17 (FIG. 1). These include 40 AA centered around each core domain, also all the possible 20 AA that can be derived around the core domain, and all the possible 10 AA that can be derived around the core domain. Also listed are multiple mutations on the 40AA, 20 AA, and 10 AA (X, X0 equal all possible AA).
(G/A)₀₋₂₀=0 to 20 amino acids of either G or A in each position.
   X0=any one of the natural and un-natural amino acids.
   X=any one of the natural and un-natural amino acids.

The amino acid in any of the position in all of the sequences can be replaced by an amino acid that belongs to the same amino acid group as listed in Table 1. (Table 1, Group 1=R, H, K; Group 2=D, E; Group 3=S, T, N, Q; Group 4=A, I, L, M, F, W, Y, V; Group 5=C, U, G, P)

TABLE 1
Twenty-One Amino Acids

| | | |
|---|---|---|
| Group 1 | Amino acids with positively | R = Arginine (Arg) |
| | charged side chain | H = Histidine (His) |
| | | K = Lysine (Lys) |
| Group 2 | Amino acids with negatively | D = Aspartic acid (Asp) |
| | charged side chain | E = Glutamic acid (Glu) |
| Group 3 | Amino acids with polar | S = Serine (Ser) |
| | uncharged side chain | T = Threonine (Thr) |
| | | N = Asparagine (Asn) |
| | | Q = Glutamine (Gln) |
| Group 4 | Amino acids with hydrophobic | A = Alanine (Ala) |
| | side chain | I = Isoleucine (Ile) |
| | | L = Leucine (Leu) |
| | | M = Methionine (Met) |
| | | F = Phenylalanine (Phe) |
| | | W = Tryptophan (Trp) |
| | | Y = Tyrosine (Tyr) |
| | | V = Valine (Val) |
| Group 5 | Amino acids with special cases | C = Cysteine (Cys) |
| | | U = Selenocysteine (Sec) |
| | | G = Glycine (Gly) |
| | | P = Proline (Pro) |

A peptide sequence of the invention includes one or more of the following:

| ID(Name) | Peptide sequence (N to C) |
|---|---|
| SEQ ID NO: 1(NB5-core): | RRRRRRRR(G/A)₀₋₂₀YNELRVAPEE |
| SEQ ID NO: 2(NB7-core): | RRRRRRRR(G/A)₀₋₂₀QIMFETFNTP |
| SEQ ID NO: 3(NB9-core): | RRRRRRRR(G/A)₀₋₂₀LPHAILRLDL |
| SEQ ID NO: 4(NB10-core): | RRRRRRRR(G/A)₀₋₂₀AGRDLTDYLM |
| SEQ ID NO: 5(B11-core): | RRRRRRRR(G/A)₀₋₂₀LCYVALDFEQ |
| SEQ ID NO: 6(B17-core): | RRRRRRRR(G/A)₀₋₂₀KYSVWIGGSI |
| SEQ ID NO: 7(B5-20AA): | RRRRRRRR(G/A)₀₋₂₀YNELRVAPEEHPVLLTEAPL |
| SEQ ID NO: 8(B6-20AA): | RRRRRRRR(G/A)₀₋₂₀NPKANREKMTQIMFETFNTP |
| SEQ ID NO: 9(N7-20AA): | RRRRRRRR(G/A)₀₋₂₀QIMFETFNTPAMYVAIQAVL |
| SEQ ID NO: 10(N9-20AA): | RRRRRRRR(G/A)₀₋₂₀HTVPIYEGYALPHAILRLDL |
| SEQ ID NO: 11(B9-20AA): | RRRRRRRR(G/A)₀₋₂₀LPHAILRLDLAGRDLTDYLM |
| SEQ ID NO: 12(N10-20AA): | RRRRRRRR(G/A)₀₋₂₀AGRDLTDYLMKILTERGYSF |
| SEQ ID NO: 13(B11-20AA): | RRRRRRRR(G/A)₀₋₂₀DIKEKLCYVALDFEQEMATA |
| SEQ ID NO: 14(B17-20AA): | RRRRRRRR(G/A)₀₋₂₀APPERKYSVWIGGSILASLS |
| SEQ ID NO: 15(NB5-V1): | (X)₀₋₂₀(X)NELRVAPEE(X)₀₋₂₀ |
| SEQ ID NO: 16(NB5-V2): | (G/A)₀₋₂₀Y(X)ELRVAPEE(G/A)₀₋₂₀ |
| SEQ ID NO: 17(NB5-V3): | (G/A)₀₋₂₀YN(X)LRVAPEE(G/A)₀₋₂₀ |
| SEQ ID NO: 18(NB5-V4): | (G/A)₀₋₂₀YNE(X)RVAPEE(G/A)₀₋₂₀ |
| SEQ ID NO: 19(NB5-V5): | (G/A)₀₋₂₀YNEL(X)VAPEE(G/A)₀₋₂₀ |
| SEQ ID NO: 20(NB5-V6): | (G/A)₀₋₂₀YNELR(X)APEE(G/A)₀₋₂₀ |
| SEQ ID NO: 21(NB5-V7): | (G/A)₀₋₂₀YNELRV(X)PEE(G/A)₀₋₂₀ |
| SEQ ID NO: 22(NB5-V8): | (G/A)₀₋₂₀YNELRVA(X)EE(G/A)₀₋₂₀ |
| SEQ ID NO: 23(NB5-V9): | (G/A)₀₋₂₀YNELRVAP(X)E(G/A)₀₋₂₀ |
| SEQ ID NO: 24(NB5-V10): | (G/A)₀₋₂₀YNELRVAPE(X)(G/A)₀₋₂₀ |
| SEQ ID NO: 25(B5-NV2): | (X)₀₋₂₀WHHTFYNELR(X)₀₋₂₀ |
| SEQ ID NO: 26(B5-NV2): | (G/A)₀₋₂₀W(X)HTFYNELR(G/A)₀₋₂₀ |
| SEQ ID NO: 27(B5-NV3): | (G/A)₀₋₂₀WH(X)TFYNELR(G/A)₀₋₂₀ |
| SEQ ID NO: 28(B5-NV4): | (G/A)₀₋₂₀WHH(X)FYNELR(G/A)₀₋₂₀ |
| SEQ ID NO: 29(B5-NV5): | (G/A)₀₋₂₀WHHT(X)YNELR(G/A)₀₋₂₀ |
| SEQ ID NO: 30(B5-NV6): | (G/A)₀₋₂₀WHHTF(X)NELR(G/A)₀₋₂₀ |
| SEQ ID NO: 31(B5-NV7): | (G/A)₀₋₂₀WHHTFY(X)ELR(G/A)₀₋₂₀ |
| SEQ ID NO: 32(B5-NV8): | (G/A)₀₋₂₀WHHTFYN(X)LR(G/A)₀₋₂₀ |
| SEQ ID NO: 33(B5-NV9): | (G/A)₀₋₂₀WHHTFYNE(X)R(G/A)₀₋₂₀ |
| SEQ ID NO: 34(B5-NV10): | (G/A)₀₋₂₀WHHTFYNEL(X)(G/A)₀₋₂₀ |
| SEQ ID NO: 35(B5-N1): | (G/A)₀₋₂₀VTNWDDMEKI(G/A)₀₋₂₀ |
| SEQ ID NO: 36(B5-N2): | (G/A)₀₋₂₀TNWDDMEKIW(G/A)₀₋₂₀ |
| SEQ ID NO: 37(B5-N3): | (G/A)₀₋₂₀NWDDMEKIWH(G/A)₀₋₂₀ |
| SEQ ID NO: 38(B5-N4): | (G/A)₀₋₂₀WDDMEKIWHH(G/A)₀₋₂₀ |
| SEQ ID NO: 39(B5-N5): | (G/A)₀₋₂₀DDMEKIWHHT(G/A)₀₋₂₀ |
| SEQ ID NO: 40(B5-N6): | (G/A)₀₋₂₀DMEKIWHHTF(G/A)₀₋₂₀ |
| SEQ ID NO: 41(B5-N7): | (G/A)₀₋₂₀MEKIWHHTFY(G/A)₀₋₂₀ |
| SEQ ID NO: 42(B5-N8): | (G/A)₀₋₂₀EKIWHHTFYN(G/A)₀₋₂₀ |
| SEQ ID NO: 43(B5-N9): | (G/A)₀₋₂₀KIWHHTFYNE(G/A)₀₋₂₀ |
| SEQ ID NO: 44(B5-N10): | (G/A)₀₋₂₀IWHHTFYNEL(G/A)₀₋₂₀ |
| SEQ ID NO: 45(B5-N11): | (G/A)₀₋₂₀WHHTFYNELR(G/A)₀₋₂₀ |
| SEQ ID NO: 46(B5-N12): | (G/A)₀₋₂₀HHTFYNELRV(G/A)₀₋₂₀ |
| SEQ ID NO: 47(B5-N13): | (G/A)₀₋₂₀HTFYNELRVA(G/A)₀₋₂₀ |
| SEQ ID NO: 48(B5-N14): | (G/A)₀₋₂₀TFYNELRVAP(G/A)₀₋₂₀ |
| SEQ ID NO: 49(B5-N15): | (G/A)₀₋₂₀FYNELRVAPE(G/A)₀₋₂₀ |
| SEQ ID NO: 50(B5-N16): | (G/A)₀₋₂₀YNELRVAPEE(G/A)₀₋₂₀ |
| SEQ ID NO: 51(B5-N17): | (G/A)₀₋₂₀NELRVAPEEH(G/A)₀₋₂₀ |
| SEQ ID NO: 52(B5-N18): | (G/A)₀₋₂₀ELRVAPEEHP(G/A)₀₋₂₀ |
| SEQ ID NO: 53(B5-N19): | (G/A)₀₋₂₀LRVAPEEHPV(G/A)₀₋₂₀ |
| SEQ ID NO: 54(B5-N20): | (G/A)₀₋₂₀RVAPEEHPVL(G/A)₀₋₂₀ |
| SEQ ID NO: 55(B5-N21): | (G/A)₀₋₂₀VAPEEHPVLL(G/A)₀₋₂₀ |
| SEQ ID NO: 56(B5-N22): | (G/A)₀₋₂₀APEEHPVLLT(G/A)₀₋₂₀ |
| SEQ ID NO: 57(B5-N23): | (G/A)₀₋₂₀PEEHPVLLTE(G/A)₀₋₂₀ |
| SEQ ID NO: 58(B5-N24): | (G/A)₀₋₂₀EEHPVLLTEA(G/A)₀₋₂₀ |
| SEQ ID NO: 59(B5-N25): | (G/A)₀₋₂₀EHPVLLTEAP(G/A)₀₋₂₀ |
| SEQ ID NO: 60(B5-N26): | (G/A)₀₋₂₀HPVLLTEAPL(G/A)₀₋₂₀ |
| SEQ ID NO: 61(B5-N27): | (G/A)₀₋₂₀PVLLTEAPLN(G/A)₀₋₂₀ |
| SEQ ID NO: 62(B5-N28): | (G/A)₀₋₂₀VLLTEAPLNP(G/A)₀₋₂₀ |
| SEQ ID NO: 63(B5-N29): | (G/A)₀₋₂₀LLTEAPLNPK(G/A)₀₋₂₀ |
| SEQ ID NO: 64(B5-N30): | (G/A)₀₋₂₀LTEAPLNPKA(G/A)₀₋₂₀ |
| SEQ ID NO: 65(B5-N31): | (G/A)₀₋₂₀TEAPLNPKAN(G/A)₀₋₂₀ |
| SEQ ID NO: 66(B5-M2): | (G/A)₀₋₂₀EHGIVTNWDDMEKIWHHTFY(G/A)₀₋₂₀ |
| SEQ ID NO: 67(B5-M3): | (G/A)₀₋₂₀HGIVTNWDDMEKIWHHTFYN(G/A)₀₋₂₀ |
| SEQ ID NO: 68(B5-M4): | (G/A)₀₋₂₀GIVTNWDDMEKIWHHTFYNE(G/A)₀₋₂₀ |
| SEQ ID NO: 69(B5-M5): | (G/A)₀₋₂₀IVTNWDDMEKIWHHTFYNEL(G/A)₀₋₂₀ |
| SEQ ID NO: 70(B5-M6): | (G/A)₀₋₂₀VTNWDDMEKIWHHTFYNELR(G/A)₀₋₂₀ |
| SEQ ID NO: 71(B5-M7): | (G/A)₀₋₂₀TNWDDMEKIWHHTFYNELRV(G/A)₀₋₂₀ |
| SEQ ID NO: 72(B5-M8): | (G/A)₀₋₂₀NWDDMEKIWHHTFYNELRVA(G/A)₀₋₂₀ |
| SEQ ID NO: 73(B5-M9): | (G/A)₀₋₂₀WDDMEKIWHHTFYNELRVAP(G/A)₀₋₂₀ |
| SEQ ID NO: 74(B5-M10): | (G/A)₀₋₂₀DDMEKIWHHTFYNELRVAPE(G/A)₀₋₂₀ |
| SEQ ID NO: 75(B5): | (G/A)₀₋₂₀DMEKIWHHTFYNELRVAPEE(G/A)₀₋₂₀ |
| SEQ ID NO: 76(B5-P1): | (G/A)₀₋₂₀MEKIWHHTFYNELRVAPEEH(G/A)₀₋₂₀ |
| SEQ ID NO: 77(B5-P2): | (G/A)₀₋₂₀EKIWHHTFYNELRVAPEEHP(G/A)₀₋₂₀ |
| SEQ ID NO: 78(B5-P3): | (G/A)₀₋₂₀KIWHHTFYNELRVAPEEHPV(G/A)₀₋₂₀ |
| SEQ ID NO: 79(B5-P4): | (G/A)₀₋₂₀IWHHTFYNELRVAPEEHPVL(G/A)₀₋₂₀ |
| SEQ ID NO: 80(B5-P5): | (G/A)₀₋₂₀WHHTFYNELRVAPEEHPVLL(G/A)₀₋₂₀ |
| SEQ ID NO: 81(B5-P6): | (G/A)₀₋₂₀HHTFYNELRVAPEEHPVLLT(G/A)₀₋₂₀ |
| SEQ ID NO: 82(B5-P7): | (G/A)₀₋₂₀HTFYNELRVAPEEHPVLLTE(G/A)₀₋₂₀ |
| SEQ ID NO: 83(B5-P8): | (G/A)₀₋₂₀TFYNELRVAPEEHPVLLTEA(G/A)₀₋₂₀ |
| SEQ ID NO: 84(B5-P9): | (G/A)₀₋₂₀FYNELRVAPEEHPVLLTEAP(G/A)₀₋₂₀ |
| SEQ ID NO: 85(B5-P10): | (G/A)₀₋₂₀YNELRVAPEEHPVLLTEAPL(G/A)₀₋₂₀ |
| SEQ ID NO: 86(B5-V1): | (G/A)₀₋₂₀(X)MEKIWHHTFYNELRVAPEE(G/A)₀₋₂₀ |
| SEQ ID NO: 87(B5-V2): | (G/A)₀₋₂₀D(X)EKIWHHTFYNELRVAPEE(G/A)₀₋₂₀ |
| SEQ ID NO: 88(B5-V3): | (G/A)₀₋₂₀DM(X)KIWHHTFYNELRVAPEE(G/A)₀₋₂₀ |
| SEQ ID NO: 89(B5-V4): | (G/A)₀₋₂₀DME(X)IWHHTFYNELRVAPEE(G/A)₀₋₂₀ |
| SEQ ID NO: 90(B5-V5): | (G/A)₀₋₂₀DMEK(X)WHHTFYNELRVAPEE(G/A)₀₋₂₀ |
| SEQ ID NO: 91(B5-V6): | (G/A)₀₋₂₀DMEKI(X)HHTFYNELRVAPEE(G/A)₀₋₂₀ |
| SEQ ID NO: 92(B5-V7): | (G/A)₀₋₂₀DMEKIW(X)HTFYNELRVAPEE(G/A)₀₋₂₀ |
| SEQ ID NO: 93(B5-V8): | (G/A)₀₋₂₀DMEKIWH(X)TFYNELRVAPEE(G/A)₀₋₂₀ |
| SEQ ID NO: 94(B5-V9): | (G/A)₀₋₂₀DMEKIWHH(X)FYNELRVAPEE(G/A)₀₋₂₀ |
| SEQ ID NO: 95(B5-V10): | (G/A)₀₋₂₀DMEKIWHHT(X)YNELRVAPEE(G/A)₀₋₂₀ |
| SEQ ID NO: 96(B5-V11): | (G/A)₀₋₂₀DMEKIWHHTF(X)NELRVAPEE(G/A)₀₋₂₀ |
| SEQ ID NO: 97(B5-V12): | (G/A)₀₋₂₀DMEKIWHHTFY(X)ELRVAPEE(G/A)₀₋₂₀ |
| SEQ ID NO: 98(B5-V13): | (G/A)₀₋₂₀DMEKIWHHTFYN(X)LRVAPEE(G/A)₀₋₂₀ |
| SEQ ID NO: 99(B5-X1): | (G/A)₀₋₂₀(X)EHGIVTNWDDMEKIWHHTFYNELRVAPEEHPVLLTEAPL(G/A)₀₋₂₀ |
| SEQ ID NO: 100(B5-X2): | (G/A)₀₋₂₀I(X)HGIVTNWDDMEKIWHHTFYNELRVAPEEHPVLLTEAPL(G/A)₀₋₂₀ |
| SEQ ID NO: 101(B5-X3): | (G/A)₀₋₂₀IE(X)GIVTNWDDMEKIWHHTFYNELRVAPEEHPVLLTEAPL(G/A)₀₋₂₀ |
| SEQ ID NO: 102(B5-X4): | (G/A)₀₋₂₀IEH(X)IVTNWDDMEKIWHHTFYNELRVAPEEHPVLLTEAPL(G/A)₀₋₂₀ |
| SEQ ID NO: 103(B5-X5): | (G/A)₀₋₂₀IEHG(X)VTNWDDMEKIWHHTFYNELRVAPEEHPVLLTEAPL(G/A)₀₋₂₀ |
| SEQ ID NO: 104(B5-X6): | (G/A)₀₋₂₀IEHGI(X)TNWDDMEKIWHHTFYNELRVAPEEHPVLLTEAPL(G/A)₀₋₂₀ |
| SEQ ID NO: 105(B5-X7): | (G/A)₀₋₂₀IEHGIV(X)NWDDMEKIWHHTFYNELRVAPEEHPVLLTEAPL(G/A)₀₋₂₀ |
| SEQ ID NO: 106(B5-X8): | (G/A)₀₋₂₀IEHGIVT(X)WDDMEKIWHHTFYNELRVAPEEHPVLLTEAPL(G/A)₀₋₂₀ |
| SEQ ID NO: 107(B5-X9): | (G/A)₀₋₂₀IEHGIVTN(X)DDMEKIWHHTFYNELRVAPEEHPVLLTEAPL(G/A)₀₋₂₀ |
| SEQ ID NO: 108(B5-X10): | (G/A)₀₋₂₀IEHGIVTNW(X)DMEKIWHHTFYNELRVAPEEHPVLLTEAPL(G/A)₀₋₂₀ |
| SEQ ID NO: 109(B5-X11): | (G/A)₀₋₂₀IEHGIVTNWD(X)MEKIWHHTFYNELRVAPEEHPVLLTEAPL(G/A)₀₋₂₀ |
| SEQ ID NO: 110(B5-X12): | (G/A)₀₋₂₀IEHGIVTNWDD(X)EKIWHHTFYNELRVAPEEHPVLLTEAPL(G/A)₀₋₂₀ |
| SEQ ID NO: 111(B5-X13): | (G/A)₀₋₂₀IEHGIVTNWDDM(X)KIWHHTFYNELRVAPEEHPVLLTEAPL(G/A)₀₋₂₀ |
| SEQ ID NO: 112(B5-X14): | (G/A)₀₋₂₀IEHGIVTNWDDME(X)IWHHTFYNELRVAPEEHPVLLTEAPL(G/A)₀₋₂₀ |
| SEQ ID NO: 113(B5-X15): | (G/A)₀₋₂₀IEHGIVTNWDDMEK(X)WHHTFYNELRVAPEEHPVLLTEAPL(G/A)₀₋₂₀ |
| SEQ ID NO: 114(B5-X16): | (G/A)₀₋₂₀IEHGIVTNWDDMEKI(X)HHTFYNELRVAPEEHPVLLTEAPL(G/A)₀₋₂₀ |
| SEQ ID NO: 115(B5-X17): | (G/A)₀₋₂₀IEHGIVTNWDDMEKIW(X)HTFYNELRVAPEEHPVLLTEAPL(G/A)₀₋₂₀ |
| SEQ ID NO: 116(B5-X18): | (G/A)₀₋₂₀IEHGIVTNWDDMEKIWH(X)TFYNELRVAPEEHPVLLTEAPL(G/A)₀₋₂₀ |
| SEQ ID NO: 117(B5-X19): | (G/A)₀₋₂₀IEHGIVTNWDDMEKIWHH(X)FYNELRVAPEEHPVLLTEAPL(G/A)₀₋₂₀ |
| SEQ ID NO: 118(B5-X20): | (G/A)₀₋₂₀IEHGIVTNWDDMEKIWHHT(X)YNELRVAPEEHPVLLTEAPL(G/A)₀₋₂₀ |
| SEQ ID NO: 119(B5-X21): | (G/A)₀₋₂₀IEHGIVTNWDDMEKIWHHTF(X)NELRVAPEEHPVLLTEAPL(G/A)₀₋₂₀ |
| SEQ ID NO: 120(B5-X22): | (G/A)₀₋₂₀IEHGIVTNWDDMEKIWHHTFY(X)ELRVAPEEHPVLLTEAPL(G/A)₀₋₂₀ |
| SEQ ID NO: 121(B5-X23): | (G/A)₀₋₂₀IEHGIVTNWDDMEKIWHHTFYN(X)LRVAPEEHPVLLTEAPL(G/A)₀₋₂₀ |
| SEQ ID NO: 122(B5-X24): | (G/A)₀₋₂₀IEHGIVTNWDDMEKIWHHTFYNE(X)RVAPEEHPVLLTEAPL(G/A)₀₋₂₀ |
| SEQ ID NO: 123(B5-X25): | (G/A)₀₋₂₀IEHGIVTNWDDMEKIWHHTFYNEL(X)VAPEEHPVLLTEAPL(G/A)₀₋₂₀ |
| SEQ ID NO: 124(B5-X26): | (G/A)₀₋₂₀IEHGIVTNWDDMEKIWHHTFYNELR(X)APEEHPVLLTEAPL(G/A)₀₋₂₀ |
| SEQ ID NO: 125(B5-X27): | (G/A)₀₋₂₀IEHGIVTNWDDMEKIWHHTFYNELRV(X)PEEHPVLLTEAPL(G/A)₀₋₂₀ |
| SEQ ID NO: 126(B5-X28): | (G/A)₀₋₂₀IEHGIVTNWDDMEKIWHHTFYNELRVA(X)EEHPVLLTEAPL(G/A)₀₋₂₀ |
| SEQ ID NO: 127(B5-X29): | (G/A)₀₋₂₀IEHGIVTNWDDMEKIWHHTFYNELRVAP(X)EHPVLLTEAPL(G/A)₀₋₂₀ |
| SEQ ID NO: 128(B5-X30): | (G/A)₀₋₂₀IEHGIVTNWDDMEKIWHHTFYNELRVAPE(X)HPVLLTEAPL(G/A)₀₋₂₀ |
| SEQ ID NO: 129(B5-X31): | (G/A)₀₋₂₀IEHGIVTNWDDMEKIWHHTFYNELRVAPEE(X)PVLLTEAPL(G/A)₀₋₂₀ |
| SEQ ID NO: 130(B5-X32): | (G/A)₀₋₂₀IEHGIVTNWDDMEKIWHHTFYNELRVAPEEH(X)VLLTEAPL(G/A)₀₋₂₀ |
| SEQ ID NO: 131(B5-X33): | (G/A)₀₋₂₀IEHGIVTNWDDMEKIWHHTFYNELRVAPEEHP(X)LLTEAPL(G/A)₀₋₂₀ |
| SEQ ID NO: 132(B5-X34): | (G/A)₀₋₂₀IEHGIVTNWDDMEKIWHHTFYNELRVAPEEHPV(X)LTEAPL(G/A)₀₋₂₀ |
| SEQ ID NO: 133(B5-X35): | (G/A)₀₋₂₀IEHGIVTNWDDMEKIWHHTFYNELRVAPEEHPVL(X)TEAPL(G/A)₀₋₂₀ |
| SEQ ID NO: 134(B5-X36): | (G/A)₀₋₂₀IEHGIVTNWDDMEKIWHHTFYNELRVAPEEHPVLL(X)EAPL(G/A)₀₋₂₀ |
| SEQ ID NO: 135(B5-X37): | (G/A)₀₋₂₀IEHGIVTNWDDMEKIWHHTFYNELRVAPEEHPVLLT(X)APL(G/A)₀₋₂₀ |
| SEQ ID NO: 136(B5-X38): | ((G/A)₀₋₂₀IEHGIVTNWDDMEKIWHHTFYNELRVAPEEHPVLLTE(X)PL(G/A)₀₋₂₀ |
| SEQ ID NO: 137(B5-X39): | (G/A)₀₋₂₀IEHGIVTNWDDMEKIWHHTFYNELRVAPEEHPVLLTEA(X)L(G/A)₀₋₂₀ |
| SEQ ID NO: 138(B5-X40): | (G/A)₀₋₂₀IEHGIVTNWDDMEKIWHHTFYNELRVAPEEHPVLLTEAP(X)(G/A)₀₋₂₀ |
| SEQ ID NO: 139(NB7-V1): | (X)₀₋₂₀(X)IMFETFNTP(X)₀₋₂₀ |
| SEQ ID NO: 140(NB7-V2): | (G/A)₀₋₂₀Q(X)MFETFNTP(G/A)₀₋₂₀ |
| SEQ ID NO: 141(NB7-V3): | (G/A)₀₋₂₀QI(X)FETFNTP(G/A)₀₋₂₀ |
| SEQ ID NO: 142(NB7-V4): | (G/A)₀₋₂₀QIM(X)ETFNTP(G/A)₀₋₂₀ |
| SEQ ID NO: 143(NB7-V5): | (G/A)₀₋₂₀QIMF(X)TFNTP(G/A)₀₋₂₀ |
| SEQ ID NO: 144(NB7-V6): | (G/A)₀₋₂₀QIMFE(X)FNTP(G/A)₀₋₂₀ |
| SEQ ID NO: 145(NB7-V7): | (G/A)₀₋₂₀QIMFET(X)NTP(G/A)₀₋₂₀ |
| SEQ ID NO: 146(NB7-V8): | (G/A)₀₋₂₀QIMFETF(X)TP(G/A)₀₋₂₀ |
| SEQ ID NO: 147(NB7-V9): | (G/A)₀₋₂₀QIMFETFN(X)P(G/A)₀₋₂₀ |
| SEQ ID NO: 148(NB7-V10): | (G/A)₀₋₂₀QIMFETFNT(X)(G/A)₀₋₂₀ |
| SEQ ID NO: 149(N7-NX1): | (X)₀₋₂₀TFN(X0)PAMYVA(X)₀₋₂₀ |
| SEQ ID NO: 150(N7-NX1): | (G/A)₀₋₂₀(X)FN(X0)PAMYVA(G/A)₀₋₂₀ |
| SEQ ID NO: 151(N7-NX2): | (G/A)₀₋₂₀T(X)N(X0)PAMYVA(G/A)₀₋₂₀ |
| SEQ ID NO: 152(N7-NX3): | (G/A)₀₋₂₀TF(X)(X0)PAMYVA(G/A)₀₋₂₀ |
| SEQ ID NO: 153(N7-NX4): | (G/A)₀₋₂₀TFN(X0)(X)AMYVA(G/A)₀₋₂₀ |
| SEQ ID NO: 154(N7-NX5): | (G/A)₀₋₂₀TFN(X0)P(X)MYVA(G/A)₀₋₂₀ |
| SEQ ID NO: 155(N7-NX6): | (G/A)₀₋₂₀TFN(X0)PA(X)YVA(G/A)₀₋₂₀ |
| SEQ ID NO: 156(N7-NX7): | (G/A)₀₋₂₀TFN(X0)PAM(X)VA(G/A)₀₋₂₀ |
| SEQ ID NO: 157(N7-NX8): | (G/A)₀₋₂₀TFN(X0)PAMY(X)A(G/A)₀₋₂₀ |
| SEQ ID NO: 158(N7-NX9): | (G/A)₀₋₂₀TFN(X0)PAMYV(X)(G/A)₀₋₂₀ |
| SEQ ID NO: 159(N7-T1): | (G/A)₀₋₂₀TEAPLNPKAN(G/A)₀₋₂₀ |
| SEQ ID NO: 160(N7-T2): | (G/A)₀₋₂₀EAPLNPKANR(G/A)₀₋₂₀ |
| SEQ ID NO: 161(N7-T3): | (G/A)₀₋₂₀APLNPKANRE(G/A)₀₋₂₀ |
| SEQ ID NO: 162(N7-T4): | (G/A)₀₋₂₀PLNPKANREK(G/A)₀₋₂₀ |
| SEQ ID NO: 163(N7-T5): | (G/A)₀₋₂₀LNPKANREKM(G/A)₀₋₂₀ |
| SEQ ID NO: 164(N7-T6): | (G/A)₀₋₂₀NPKANREKMT(G/A)₀₋₂₀ |
| SEQ ID NO: 165(N7-T7): | (G/A)₀₋₂₀PKANREKMTQ(G/A)₀₋₂₀ |
| SEQ ID NO: 166(N7-T8): | (G/A)₀₋₂₀KANREKMTQI(G/A)₀₋₂₀ |
| SEQ ID NO: 167(N7-T9): | (G/A)₀₋₂₀ANREKMTQIM(G/A)₀₋₂₀ |
| SEQ ID NO: 168(N7-T10): | (G/A)₀₋₂₀NREKMTQIMF(G/A)₀₋₂₀ |
| SEQ ID NO: 169(N7-T11): | (G/A)₀₋₂₀REKMTQIMFE(G/A)₀₋₂₀ |
| SEQ ID NO: 170(N7-T12): | (G/A)₀₋₂₀EKMTQIMFET(G/A)₀₋₂₀ |
| SEQ ID NO: 171(N7-T13): | (G/A)₀₋₂₀KMTQIMFETF(G/A)₀₋₂₀ |
| SEQ ID NO: 172(N7-T14): | (G/A)₀₋₂₀MTQIMFETFN(G/A)₀₋₂₀ |
| SEQ ID NO: 173(N7-T15): | (G/A)₀₋₂₀TQIMFETFN(X0)(G/A)₀₋₂₀ |
| SEQ ID NO: 174(N7-T16): | (G/A)₀₋₂₀QIMFETFN(X0)P(G/A)₀₋₂₀ |
| SEQ ID NO: 175(N7-T17): | (G/A)₀₋₂₀IMFETFN(X0)PA(G/A)₀₋₂₀ |
| SEQ ID NO: 176(N7-T18): | (G/A)₀₋₂₀MFETFN(X0)PAM(G/A)₀₋₂₀ |
| SEQ ID NO: 177(N7-T19): | (G/A)₀₋₂₀FETFN(X0)PAMY(G/A)₀₋₂₀ |
| SEQ ID NO: 178(N7-T20): | (G/A)₀₋₂₀ETFN(X0)PAMYV(G/A)₀₋₂₀ |
| SEQ ID NO: 179(N7-T21): | (G/A)₀₋₂₀TFN(X0)PAMYVA(G/A)₀₋₂₀ |
| SEQ ID NO: 180(N7-T22): | (G/A)₀₋₂₀FN(X0)PAMYVAI(G/A)₀₋₂₀ |
| SEQ ID NO: 181(N7-T23): | (G/A)₀₋₂₀N(X0)PAMYVAIQ(G/A)₀₋₂₀ |
| SEQ ID NO: 182(N7-T24): | (G/A)₀₋₂₀(X0)PAMYVAIQA(G/A)₀₋₂₀ |
| SEQ ID NO: 183(N7-T25): | (G/A)₀₋₂₀PAMYVAIQAV(G/A)₀₋₂₀ |
| SEQ ID NO: 184(N7-T26): | (G/A)₀₋₂₀AMYVAIQAVL(G/A)₀₋₂₀ |
| SEQ ID NO: 185(N7-T27): | (G/A)₀₋₂₀MYVAIQAVLS(G/A)₀₋₂₀ |
| SEQ ID NO: 186(N7-T28): | (G/A)₀₋₂₀YVAIQAVLSL(G/A)₀₋₂₀ |
| SEQ ID NO: 187(N7-T29): | (G/A)₀₋₂₀VAIQAVLSLY(G/A)₀₋₂₀ |
| SEQ ID NO: 188(N7-T30): | (G/A)₀₋₂₀AIQAVLSLYA(G/A)₀₋₂₀ |
| SEQ ID NO: 189(N7-T31): | (G/A)₀₋₂₀IQAVLSLYAS(G/A)₀₋₂₀ |
| SEQ ID NO: 190(N7-T32): | (G/A)₀₋₂₀QAVLSLYASG(G/A)₀₋₂₀ |
| SEQ ID NO: 191(N7-M1): | (G/A)₀₋₂₀TEAPLNPKANREKMTQIMFE(G/A)₀₋₂₀ |
| SEQ ID NO: 192(N7-M2): | (G/A)₀₋₂₀EAPLNPKANREKMTQIMFET(G/A)₀₋₂₀ |
| SEQ ID NO: 193(N7-M3): | (G/A)₀₋₂₀APLNPKANREKMTQIMFETF(G/A)₀₋₂₀ |
| SEQ ID NO: 194(N7-M4): | (G/A)₀₋₂₀PLNPKANREKMTQIMFETFN(G/A)₀₋₂₀ |
| SEQ ID NO: 195(N7-M5): | (G/A)₀₋₂₀LNPKANREKMTQIMFETFN(X0)(G/A)₀₋₂₀ |
| SEQ ID NO: 196(N7-M6): | (G/A)₀₋₂₀NPKANREKMTQIMFETFN(X0)P(G/A)₀₋₂₀ |
| SEQ ID NO: 197(N7-M7): | (G/A)₀₋₂₀PKANREKMTQIMFETFN(X0)PA(G/A)₀₋₂₀ |
| SEQ ID NO: 198(N7-M8): | (G/A)₀₋₂₀KANREKMTQIMFETFN(X0)PAM(G/A)₀₋₂₀ |
| SEQ ID NO: 199(N7-M9): | (G/A)₀₋₂₀ANREKMTQIMFETFN(X0)PAMY(G/A)₀₋₂₀ |
| SEQ ID NO: 200(N7-M10): | (G/A)₀₋₂₀NREKMTQIMFETFN(X0)PAMYV(G/A)₀₋₂₀ |
| SEQ ID NO: 201(N7-M11): | (G/A)₀₋₂₀REKMTQIMFETFN(X0)PAMYVA(G/A)₀₋₂₀ |
| SEQ ID NO: 202(N7-M12): | (G/A)₀₋₂₀EKMTQIMFETFN(X0)PAMYVAI(G/A)₀₋₂₀ |
| SEQ ID NO: 203(N7-M13): | (G/A)₀₋₂₀KMTQIMFETFN(X0)PAMYVAIQ(G/A)₀₋₂₀ |
| SEQ ID NO: 204(N7-M14): | (G/A)₀₋₂₀MTQIMFETFN(X0)PAMYVAIQA(G/A)₀₋₂₀ |
| SEQ ID NO: 205(N7-M15): | (G/A)₀₋₂₀TQIMFETFN(X0)PAMYVAIQAV(G/A)₀₋₂₀ |
| SEQ ID NO: 206(N7): | (G/A)₀₋₂₀QIMFETFN(X0)PAMYVAIQAVL(G/A)₀₋₂₀ |
| SEQ ID NO: 207(N7-P1): | (G/A)₀₋₂₀IMFETFN(X0)PAMYVAIQAVLS(G/A)₀₋₂₀ |
| SEQ ID NO: 208(N7-P2): | (G/A)₀₋₂₀MFETFN(X0)PAMYVAIQAVLSL(G/A)₀₋₂₀ |
| SEQ ID NO: 209(N7-P3): | (G/A)₀₋₂₀FETFN(X0)PAMYVAIQAVLSLY(G/A)₀₋₂₀ |
| SEQ ID NO: 210(N7-P4): | (G/A)₀₋₂₀ETFN(X0)PAMYVAIQAVLSLYA(G/A)₀₋₂₀ |
| SEQ ID NO: 211(N7-P5): | (G/A)₀₋₂₀TFN(X0)PAMYVAIQAVLSLYAS(G/A)₀₋₂₀ |
| SEQ ID NO: 212(N7-V1): | (G/A)₀₋₂₀(X)QIMFETFN(X0)PAMYVAIQAVL(G/A)₀₋₂₀ |
| SEQ ID NO: 213(N7-V2): | (G/A)₀₋₂₀Q(X)IMFETFN(X0)PAMYVAIQAVL(G/A)₀₋₂₀ |
| SEQ ID NO: 214(N7-V3): | (G/A)₀₋₂₀QI(X)MFETFN(X0)PAMYVAIQAVL(G/A)₀₋₂₀ |
| SEQ ID NO: 215(N7-V4): | (G/A)₀₋₂₀QIM(X)FETFN(X0)PAMYVAIQAVL(G/A)₀₋₂₀ |
| SEQ ID NO: 216(N7-V5): | (G/A)₀₋₂₀QIMF(X)ETFN(X0)PAMYVAIQAVL(G/A)₀₋₂₀ |
| SEQ ID NO: 217(N7-V6): | (G/A)₀₋₂₀QIMFE(X)TFN(X0)PAMYVAIQAVL(G/A)₀₋₂₀ |
| SEQ ID NO: 218(N7-V7): | (G/A)₀₋₂₀QIMFET(X)FN(X0)PAMYVAIQAVL(G/A)₀₋₂₀ |
| SEQ ID NO: 219(N7-V8): | (G/A)₀₋₂₀QIMFETF(X)N(X0)PAMYVAIQAVL(G/A)₀₋₂₀ |
| SEQ ID NO: 220(N7-V9): | (G/A)₀₋₂₀QIMFETFN(X)(X0)PAMYVAIQAVL(G/A)₀₋₂₀ |
| SEQ ID NO: 221(N7-V10): | (G/A)₀₋₂₀QIMFETFN(X0)P(X)AMYVAIQAVL(G/A)₀₋₂₀ |
| SEQ ID NO: 222(N7-V11): | (G/A)₀₋₂₀QIMFETFN(X0)PA(X)MYVAIQAVL(G/A)₀₋₂₀ |
| SEQ ID NO: 223(N7-V12): | (G/A)₀₋₂₀QIMFETFN(X0)PAM(X)YVAIQAVL(G/A)₀₋₂₀ |
| SEQ ID NO: 224(N7-V13): | (G/A)₀₋₂₀QIMFETFN(X0)PAMY(X)VAIQAVL(G/A)₀₋₂₀ |
| SEQ ID NO: 225(N7-V14): | (G/A)₀₋₂₀QIMFETFN(X0)PAMYV(X)AIQAVL(G/A)₀₋₂₀ |
| SEQ ID NO: 226(N7-V15): | (G/A)₀₋₂₀QIMFETFN(X0)PAMYVA(X)IQAVL(G/A)₀₋₂₀ |
| SEQ ID NO: 227(N7-V16): | (G/A)₀₋₂₀QIMFETFN(X0)PAMYVAI(X)QAVL(G/A)₀₋₂₀ |
| SEQ ID NO: 228(N7-V17): | (G/A)₀₋₂₀QIMFETFN(X0)PAMYVAIQ(X)AVL(G/A)₀₋₂₀ |
| SEQ ID NO: 229(N7-V18): | (G/A)₀₋₂₀QIMFETFN(X0)PAMYVAIQA(X)VL(G/A)₀₋₂₀ |
| SEQ ID NO: 230(N7-V19): | (G/A)₀₋₂₀QIMFETFN(X0)PAMYVAIQAV(X)L(G/A)₀₋₂₀ |
| SEQ ID NO: 231(N7-V20): | (G/A)₀₋₂₀QIMFETFN(X0)PAMYVAIQAVL(X)(G/A)₀₋₂₀ |
| SEQ ID NO: 232(N7-X1): | (G/A)₀₋₂₀(X)PKANREKMTQIMFETFN(X0)PAMYVAIQAVLSLYASGRTTGI(G/A)₀₋₂₀ |
| SEQ ID NO: 233(N7-X2): | (G/A)₀₋₂₀N(X)KANREKMTQIMFETFN(X0)PAMYVAIQAVLSLYASGRTTGI(G/A)₀₋₂₀ |
| SEQ ID NO: 234(N7-X3): | (G/A)₀₋₂₀NP(X)ANREKMTQIMFETFN(X0)PAMYVAIQAVLSLYASGRTTGI(G/A)₀₋₂₀ |
| SEQ ID NO: 235(N7-X4): | (G/A)₀₋₂₀NPK(X)NREKMTQIMFETFN(X0)PAMYVAIQAVLSLYASGRTTGI(G/A)₀₋₂₀ |
| SEQ ID NO: 236(N7-X5): | (G/A)₀₋₂₀NPKA(X)REKMTQIMFETFN(X0)PAMYVAIQAVLSLYASGRTTGI(G/A)₀₋₂₀ |
| SEQ ID NO: 237(N7-X6): | (G/A)₀₋₂₀NPKAN(X)EKMTQIMFETFN(X0)PAMYVAIQAVLSLYASGRTTGI(G/A)₀₋₂₀ |
| SEQ ID NO: 238(N7-X7): | (G/A)₀₋₂₀NPKANR(X)KMTQIMFETFN(X0)PAMYVAIQAVLSLYASGRTTGI(G/A)₀₋₂₀ |
| SEQ ID NO: 239(N7-X8): | (G/A)₀₋₂₀NPKANRE(X)MTQIMFETFN(X0)PAMYVAIQAVLSLYASGRTTGI(G/A)₀₋₂₀ |
| SEQ ID NO: 240(N7-X9): | (G/A)₀₋₂₀NPKANREK(X)TQIMFETFN(X0)PAMYVAIQAVLSLYASGRTTGI(G/A)₀₋₂₀ |
| SEQ ID NO: 241(N7-X10): | (G/A)₀₋₂₀NPKANREKM(X)QIMFETFN(X0)PAMYVAIQAVLSLYASGRTTGI(G/A)₀₋₂₀ |
| SEQ ID NO: 242(N7-X11): | (G/A)₀₋₂₀NPKANREKMT(X)IMFETFN(X0)PAMYVAIQAVLSLYASGRTTGI(G/A)₀₋₂₀ |
| SEQ ID NO: 243(N7-X12): | (G/A)₀₋₂₀NPKANREKMTQ(X)MFETFN(X0)PAMYVAIQAVLSLYASGRTTGI(G/A)₀₋₂₀ |
| SEQ ID NO: 244(N7-X13): | (G/A)₀₋₂₀NPKANREKMTQI(X)FETFN(X0)PAMYVAIQAVLSLYASGRTTGI(G/A)₀₋₂₀ |
| SEQ ID NO: 245(N7-X14): | (G/A)₀₋₂₀NPKANREKMTQIM(X)ETFN(X0)PAMYVAIQAVLSLYASGRTTGI(G/A)₀₋₂₀ |
| SEQ ID NO: 246(N7-X15): | (G/A)₀₋₂₀NPKANREKMTQIMF(X)TFN(X0)PAMYVAIQAVLSLYASGRTTGI(G/A)₀₋₂₀ |
| SEQ ID NO: 247(N7-X16): | (G/A)₀₋₂₀NPKANREKMTQIMFE(X)FN(X0)PAMYVAIQAVLSLYASGRTTGI(G/A)₀₋₂₀ |
| SEQ ID NO: 248(N7-X17): | (G/A)₀₋₂₀NPKANREKMTQIMFET(X)N(X0)PAMYVAIQAVLSLYASGRTTGI(G/A)₀₋₂₀ |
| SEQ ID NO: 249(N7-X18): | (G/A)₀₋₂₀NPKANREKMTQIMFETF(X)(X0)PAMYVAIQAVLSLYASGRTTGI(G/A)₀₋₂₀ |
| SEQ ID NO: 250(N7-X19): | (G/A)₀₋₂₀NPKANREKMTQIMFETFN(X)(X0)AMYVAIQAVLSLYASGRTTGI(G/A)₀₋₂₀ |
| SEQ ID NO: 251(N7-X20): | (G/A)₀₋₂₀NPKANREKMTQIMFETFN(X)P(X0)MYVAIQAVLSLYASGRTTGI(G/A)₀₋₂₀ |
| SEQ ID NO: 252(N7-X21): | (G/A)₀₋₂₀NPKANREKMTQIMFETFN(X)PA(X0)YVAIQAVLSLYASGRTTGI(G/A)₀₋₂₀ |
| SEQ ID NO: 253(N7-X22): | (G/A)₀₋₂₀NPKANREKMTQIMFETFN(X)PAM(X0)VAIQAVLSLYASGRTTGI(G/A)₀₋₂₀ |
| SEQ ID NO: 254(N7-X23): | (G/A)₀₋₂₀NPKANREKMTQIMFETFN(X)PAMY(X0)AIQAVLSLYASGRTTGI(G/A)₀₋₂₀ |
| SEQ ID NO: 255(N7-X24): | (G/A)₀₋₂₀NPKANREKMTQIMFETFN(X)PAMYV(X0)IQAVLSLYASGRTTGI(G/A)₀₋₂₀ |
| SEQ ID NO: 256(N7-X25): | (G/A)₀₋₂₀NPKANREKMTQIMFETFN(X)PAMYVA(X0)QAVLSLYASGRTTGI(G/A)₀₋₂₀ |
| SEQ ID NO: 257(N7-X26): | (G/A)₀₋₂₀NPKANREKMTQIMFETFN(X)PAMYVAI(X0)AVLSLYASGRTTGI(G/A)₀₋₂₀ |
| SEQ ID NO: 258(N7-X27): | (G/A)₀₋₂₀NPKANREKMTQIMFETFN(X)PAMYVAIQ(X0)VLSLYASGRTTGI(G/A)₀₋₂₀ |
| SEQ ID NO: 259(N7-X28): | (G/A)₀₋₂₀NPKANREKMTQIMFETFN(X)PAMYVAIQA(X0)LSLYASGRTTGI(G/A)₀₋₂₀ |
| SEQ ID NO: 260(N7-X29): | (G/A)₀₋₂₀NPKANREKMTQIMFETFN(X)PAMYVAIQAV(X0)SLYASGRTTGI(G/A)₀₋₂₀ |
| SEQ ID NO: 261(N7-X30): | (G/A)₀₋₂₀NPKANREKMTQIMFETFN(X)PAMYVAIQAVL(X0)LYASGRTTGI(G/A)₀₋₂₀ |
| SEQ ID NO: 262(N7-X31): | (G/A)₀₋₂₀NPKANREKMTQIMFETFN(X)PAMYVAIQAVLS(X0)YASGRTTGI(G/A)₀₋₂₀ |
| SEQ ID NO: 263(N7-X32): | (G/A)₀₋₂₀NPKANREKMTQIMFETFN(X)PAMYVAIQAVLSL(X0)ASGRTTGI(G/A)₀₋₂₀ |
| SEQ ID NO: 264(N7-X33): | (G/A)₀₋₂₀NPKANREKMTQIMFETFN(X)PAMYVAIQAVLSLY(X0)SGRTTGI(G/A)₀₋₂₀ |
| SEQ ID NO: 265(N7-X34): | (G/A)₀₋₂₀NPKANREKMTQIMFETFN(X)PAMYVAIQAVLSLYA(X0)GRTTGI(G/A)₀₋₂₀ |
| SEQ ID NO: 266(N7-X35): | (G/A)₀₋₂₀NPKANREKMTQIMFETFN(X)PAMYVAIQAVLSLYAS(X0)RTTGI(G/A)₀₋₂₀ |
| SEQ ID NO: 267(N7-X36): | (G/A)₀₋₂₀NPKANREKMTQIMFETFN(X)PAMYVAIQAVLSLYASG(X0)TTGI(G/A)₀₋₂₀ |
| SEQ ID NO: 268(N7-X37): | (G/A)₀₋₂₀NPKANREKMTQIMFETFN(X)PAMYVAIQAVLSLYASGR(X0)TGI(G/A)₀₋₂₀ |
| SEQ ID NO: 269(N7-X38): | (G/A)₀₋₂₀NPKANREKMTQIMFETFN(X)PAMYVAIQAVLSLYASGRT(X0)GI(G/A)₀₋₂₀ |
| SEQ ID NO: 270(N7-X39): | (G/A)₀₋₂₀NPKANREKMTQIMFETFN(X)PAMYVAIQAVLSLYASGRTT(X0)I(G/A)₀₋₂₀ |
| SEQ ID NO: 271(N7-X40): | (G/A)₀₋₂₀NPKANREKMTQIMFETFN(X)PAMYVAIQAVLSLYASGRTTG(X0)(G/A)₀₋₂₀ |
| SEQ ID NO: 272(B6-V1): | (G/A)₀₋₂₀(X)PKANREKMTQIMFETFN(X0)P(G/A)₀₋₂₀ |
| SEQ ID NO: 273(B6-V1): | (G/A)₀₋₂₀N(X)KANREKMTQIMFETFN(X0)P(G/A)₀₋₂₀ |
| SEQ ID NO: 274(B6-V1): | (G/A)₀₋₂₀NP(X)ANREKMTQIMFETFN(X0)P(G/A)₀₋₂₀ |
| SEQ ID NO: 275(B6-V1): | (G/A)₀₋₂₀NPK(X)NREKMTQIMFETFN(X0)P(G/A)₀₋₂₀ |
| SEQ ID NO: 276(B6-V1): | (G/A)₀₋₂₀NPKA(X)REKMTQIMFETFN(X0)P(G/A)₀₋₂₀ |
| SEQ ID NO: 277(B6-V1): | (G/A)₀₋₂₀NPKAN(X)EKMTQIMFETFN(X0)P(G/A)₀₋₂₀ |
| SEQ ID NO: 278(B6-V1): | (G/A)₀₋₂₀NPKANR(X)KMTQIMFETFN(X0)P(G/A)₀₋₂₀ |
| SEQ ID NO: 279(B6-V1): | (G/A)₀₋₂₀NPKANRE(X)MTQIMFETFN(X0)P(G/A)₀₋₂₀ |
| SEQ ID NO: 280(B6-V1): | (G/A)₀₋₂₀NPKANREK(X)TQIMFETFN(X0)P(G/A)₀₋₂₀ |
| SEQ ID NO: 281(B6-V1): | (G/A)₀₋₂₀NPKANREKM(X)QIMFETFN(X0)P(G/A)₀₋₂₀ |
| SEQ ID NO: 282(B6-V1): | (G/A)₀₋₂₀NPKANREKMT(X)IMFETFN(X0)P(G/A)₀₋₂₀ |
| SEQ ID NO: 283(B6-V1): | (G/A)₀₋₂₀NPKANREKMTQ(X)MFETFN(X0)P(G/A)₀₋₂₀ |
| SEQ ID NO: 284(B6-V1): | (G/A)₀₋₂₀NPKANREKMTQI(X)FETFN(X0)P(G/A)₀₋₂₀ |
| SEQ ID NO: 285(B6-V1): | (G/A)₀₋₂₀NPKANREKMTQIM(X)ETFN(X0)P(G/A)₀₋₂₀ |
| SEQ ID NO: 286(B6-V1): | (G/A)₀₋₂₀NPKANREKMTQIMF(X)TFN(X0)P(G/A)₀₋₂₀ |
| SEQ ID NO: 287(B6-V1): | (G/A)₀₋₂₀NPKANREKMTQIMFE(X)FN(X0)P(G/A)₀₋₂₀ |
| SEQ ID NO: 288(B6-V1): | (G/A)₀₋₂₀NPKANREKMTQIMFET(X)N(X0)P(G/A)₀₋₂₀ |
| SEQ ID NO: 289(B6-V1): | (G/A)₀₋₂₀NPKANREKMTQIMFETF(X)(X0)P(G/A)₀₋₂₀ |
| SEQ ID NO: 290(B6-V1): | (G/A)₀₋₂₀NPKANREKMTQIMFETFN(X0)(X)(G/A)₀₋₂₀ |
| SEQ ID NO: 291(NB9-core): | (X)₀₋₂₀(X)PHAILRLDL(X)₀₋₂₀ |
| SEQ ID NO: 292(NB9-core): | (G/A)₀₋₂₀L(X)HAILRLDL(G/A)₀₋₂₀ |
| SEQ ID NO: 293(NB9-core): | (G/A)₀₋₂₀LP(X)AILRLDL(G/A)₀₋₂₀ |
| SEQ ID NO: 294(NB9-core): | (G/A)₀₋₂₀LPH(X)ILRLDL(G/A)₀₋₂₀ |
| SEQ ID NO: 295(NB9-core): | (G/A)₀₋₂₀LPHA(X)LRLDL(G/A)₀₋₂₀ |
| SEQ ID NO: 296(NB9-core): | (G/A)₀₋₂₀LPHAI(X)RLDL(G/A)₀₋₂₀ |
| SEQ ID NO: 297(NB9-core): | (G/A)₀₋₂₀LPHAIL(X)LDL(G/A)₀₋₂₀ |
| SEQ ID NO: 298(NB9-core): | (G/A)₀₋₂₀LPHAILRL(X)L(G/A)₀₋₂₀ |
| SEQ ID NO: 299(NB9-core): | (G/A)₀₋₂₀LPHAILRLD(X)(G/A)₀₋₂₀ |
| SEQ ID NO: 300(N9-NV1): | (X)₀₋₂₀(X)YALPHAI(X0)R(X)₀₋₂₀ |
| SEQ ID NO: 301(N9-NV2): | (G/A)₀₋₂₀G(X)ALPHAI(X0)R(G/A)₀₋₂₀ |
| SEQ ID NO: 302(N9-NV3): | (G/A)₀₋₂₀GY(X)LPHAI(X0)R(G/A)₀₋₂₀ |
| SEQ ID NO: 303(N9-NV4): | (G/A)₀₋₂₀GYA(X)PHAI(X0)R(G/A)₀₋₂₀ |
| SEQ ID NO: 304(N9-NV5): | (G/A)₀₋₂₀GYAL(X)HAI(X0)R(G/A)₀₋₂₀ |
| SEQ ID NO: 305(N9-NV6): | (G/A)₀₋₂₀GYALP(X)AI(X0)R(G/A)₀₋₂₀ |
| SEQ ID NO: 306(N9-NV7): | (G/A)₀₋₂₀GYALPH(X)I(X0)R(G/A)₀₋₂₀ |
| SEQ ID NO: 307(N9-NV8): | (G/A)₀₋₂₀GYALPHA(X)(X0)R(G/A)₀₋₂₀ |
| SEQ ID NO: 308(N9-NV9): | (G/A)₀₋₂₀GYALPHAI(X0)(X)(G/A)₀₋₂₀ |
| SEQ ID NO: 309(N9-N5): | (G/A)₀₋₂₀GDGVTH(X0)VPI(G/A)₀₋₂₀ |
| SEQ ID NO: 310(N9-N6): | (G/A)₀₋₂₀DGVTH(X0)VPIY(G/A)₀₋₂₀ |
| SEQ ID NO: 311(N9-N7): | (G/A)₀₋₂₀GVTH(X0)VPIYE(G/A)₀₋₂₀ |
| SEQ ID NO: 312(N9-N8): | (G/A)₀₋₂₀VTH(X0)VPIYEG(G/A)₀₋₂₀ |
| SEQ ID NO: 313(N9-N9): | (G/A)₀₋₂₀TH(X0)VPIYEGY(G/A)₀₋₂₀ |
| SEQ ID NO: 314(N9-N10): | (G/A)₀₋₂₀H(X0)VPIYEGYA(G/A)₀₋₂₀ |
| SEQ ID NO: 315(N9-N11): | (G/A)₀₋₂₀(X0)VPIYEGYAL(G/A)₀₋₂₀ |
| SEQ ID NO: 316(N9-N12): | (G/A)₀₋₂₀(X0)PIYEGYALP(G/A)₀₋₂₀ |
| SEQ ID NO: 317(N9-N13): | (G/A)₀₋₂₀(X0)IYEGYALPH(G/A)₀₋₂₀ |
| SEQ ID NO: 318(N9-N14): | (G/A)₀₋₂₀(X0)YEGYALPHA(G/A)₀₋₂₀ |
| SEQ ID NO: 319(N9-N15): | (G/A)₀₋₂₀(X0)EGYALPHAI(G/A)₀₋₂₀ |
| SEQ ID NO: 320(N9-N16): | (G/A)₀₋₂₀EGYALPHAI(X0)(G/A)₀₋₂₀ |
| SEQ ID NO: 321(N9-N17): | (G/A)₀₋₂₀GYALPHAI(X0)R(G/A)₀₋₂₀ |
| SEQ ID NO: 322(N9-N18): | (G/A)₀₋₂₀YALPHAI(X0)RL(G/A)₀₋₂₀ |
| SEQ ID NO: 323(N9-N19): | (G/A)₀₋₂₀ALPHAI(X0)RLD(G/A)₀₋₂₀ |
| SEQ ID NO: 324(N9-N20): | (G/A)₀₋₂₀LPHAI(X0)RLDL(G/A)₀₋₂₀ |
| SEQ ID NO: 325(N9-N21): | (G/A)₀₋₂₀PHAI(X0)RLDLA(G/A)₀₋₂₀ |
| SEQ ID NO: 326(N9-N22): | (G/A)₀₋₂₀HAI(X0)RLDLAG(G/A)₀₋₂₀ |
| SEQ ID NO: 327(N9-N23): | (G/A)₀₋₂₀AI(X0)RLDLAGR(G/A)₀₋₂₀ |
| SEQ ID NO: 328(N9-N24): | (G/A)₀₋₂₀I(X0)RLDLAGRD(G/A)₀₋₂₀ |
| SEQ ID NO: 329(N9-N25): | (G/A)₀₋₂₀(X0)RLDLAGRDL(G/A)₀₋₂₀ |
| SEQ ID NO: 330(N9-N26): | (G/A)₀₋₂₀LDLAGRDLTD(G/A)₀₋₂₀ |
| SEQ ID NO: 331(N9-N27): | (G/A)₀₋₂₀DLAGRDLTDY(G/A)₀₋₂₀ |
| SEQ ID NO: 332(N9-N28): | (G/A)₀₋₂₀LAGRDLTDYL(G/A)₀₋₂₀ |
| SEQ ID NO: 333(N9-N29): | (G/A)₀₋₂₀AGRDLTDYLM(G/A)₀₋₂₀ |
| SEQ ID NO: 334(N9-N30): | (G/A)₀₋₂₀GRDLTDYLMK(G/A)₀₋₂₀ |
| SEQ ID NO: 335(N9-N31): | (G/A)₀₋₂₀RDLTDYLMKI(G/A)₀₋₂₀ |
| SEQ ID NO: 336(N9-N32): | (G/A)₀₋₂₀DLTDYLMKIL(G/A)₀₋₂₀ |
| SEQ ID NO: 337(N9-N32): | (G/A)₀₋₂₀LTDYLMKILT(G/A)₀₋₂₀ |
| SEQ ID NO: 338(N9-N32): | (G/A)₀₋₂₀TDYLMKILTE(G/A)₀₋₂₀ |
| SEQ ID NO: 339(N9-N32): | (G/A)₀₋₂₀DYLMKILTER(G/A)₀₋₂₀ |
| SEQ ID NO: 340(B9-T28): | (G/A)₀₋₂₀YLMKILTERG(G/A)₀₋₂₀ |
| SEQ ID NO: 341(B9-T29): | (G/A)₀₋₂₀LMKILTERGY(G/A)₀₋₂₀ |
| SEQ ID NO: 342(B9-T30): | (G/A)₀₋₂₀MKILTERGYS(G/A)₀₋₂₀ |
| SEQ ID NO: 343(B9-T31): | (G/A)₀₋₂₀KILTERGYSF(G/A)₀₋₂₀ |
| SEQ ID NO: 344(N9-V1): | (G/A)₀₋₂₀(X)VPIYEGYALPHAI(X0)RLDLAG(G/A)₀₋₂₀ |
| SEQ ID NO: 345(N9-V2): | (G/A)₀₋₂₀(X)PIYEGYALPHAI(X0)RLDLAG(G/A)₀₋₂₀ |
| SEQ ID NO: 346(N9-V3): | (G/A)₀₋₂₀V(X)IYEGYALPHAI(X0)RLDLAG(G/A)₀₋₂₀ |
| SEQ ID NO: 347(N9-V4): | (G/A)₀₋₂₀VP(X)YEGYALPHAI(X0)RLDLAG(G/A)₀₋₂₀ |
| SEQ ID NO: 348(N9-V5): | (G/A)₀₋₂₀VPI(X)EGYALPHAI(X0)RLDLAG(G/A)₀₋₂₀ |
| SEQ ID NO: 349(N9-V6): | (G/A)₀₋₂₀VPIY(X)GYALPHAI(X0)RLDLAG(G/A)₀₋₂₀ |
| SEQ ID NO: 350(N9-V7): | (G/A)₀₋₂₀VPIYE(X)YALPHAI(X0)RLDLAG(G/A)₀₋₂₀ |
| SEQ ID NO: 351(N9-V8): | (G/A)₀₋₂₀VPIYEG(X)ALPHAI(X0)RLDLAG(G/A)₀₋₂₀ |
| SEQ ID NO: 352(N9-V9): | (G/A)₀₋₂₀VPIYEGY(X)LPHAI(X0)RLDLAG(G/A)₀₋₂₀ |
| SEQ ID NO: 353(N9-V10): | (G/A)₀₋₂₀VPIYEGYA(X)PHAI(X0)RLDLAG(G/A)₀₋₂₀ |
| SEQ ID NO: 354(N9-V11): | (G/A)₀₋₂₀VPIYEGYAL(X)HAI(X0)RLDLAG(G/A)₀₋₂₀ |
| SEQ ID NO: 355(N9-V12): | (G/A)₀₋₂₀VPIYEGYALP(X)AI(X0)RLDLAG(G/A)₀₋₂₀ |
| SEQ ID NO: 356(N9-V13): | (G/A)₀₋₂₀VPIYEGYALPH(X)I(X0)RLDLAG(G/A)₀₋₂₀ |
| SEQ ID NO: 357(N9-V14): | (G/A)₀₋₂₀VPIYEGYALPHA(X)(X0)RLDLAG(G/A)₀₋₂₀ |
| SEQ ID NO: 358(N9-V15): | (G/A)₀₋₂₀VPIYEGYALPHAI(X0)(X)LDLAG(G/A)₀₋₂₀ |
| SEQ ID NO: 359(N9-V16): | (G/A)₀₋₂₀VPIYEGYALPHAI(X0)R(X)DLAG(G/A)₀₋₂₀ |
| SEQ ID NO: 360(N9-V17): | (G/A)₀₋₂₀VPIYEGYALPHAI(X0)RL(X)LAG(G/A)₀₋₂₀ |
| SEQ ID NO: 361(N9-V18): | (G/A)₀₋₂₀VPIYEGYALPHAI(X0)RLD(X)AG(G/A)₀₋₂₀ |
| SEQ ID NO: 362(N9-V19): | (G/A)₀₋₂₀VPIYEGYALPHAI(X0)RLDL(X)G(G/A)₀₋₂₀ |
| SEQ ID NO: 363(N9-V20): | (G/A)₀₋₂₀VPIYEGYALPHAI(X0)RLDLA(X)(G/A)₀₋₂₀ |
| SEQ ID NO: 364(B9-V1): | (G/A)₀₋₂₀(X)PHAI(X0)RLDLAGRDLTDYLM(G/A)₀₋₂₀ |
| SEQ ID NO: 365(B9-V2): | (G/A)₀₋₂₀L(X)HAI(X0)RLDLAGRDLTDYLM(G/A)₀₋₂₀ |
| SEQ ID NO: 366(B9-V3): | (G/A)₀₋₂₀LP(X)AI(X0)RLDLAGRDLTDYLM(G/A)₀₋₂₀ |
| SEQ ID NO: 367(B9-V4): | (G/A)₀₋₂₀LPH(X)I(X0)RLDLAGRDLTDYLM(G/A)₀₋₂₀ |
| SEQ ID NO: 368(B9-V5): | (G/A)₀₋₂₀LPHA(X)(X0)RLDLAGRDLTDYLM(G/A)₀₋₂₀ |
| SEQ ID NO: 369(B9-V6): | (G/A)₀₋₂₀LPHAI(X0)(X)LDLAGRDLTDYLM(G/A)₀₋₂₀ |
| SEQ ID NO: 370(B9-V7): | (G/A)₀₋₂₀LPHAI(X0)R(X)DLAGRDLTDYLM(G/A)₀₋₂₀ |
| SEQ ID NO: 371(B9-V8): | (G/A)₀₋₂₀LPHAI(X0)RL(X)LAGRDLTDYLM(G/A)₀₋₂₀ |
| SEQ ID NO: 372(B9-V9): | (G/A)₀₋₂₀LPHAI(X0)RLD(X)AGRDLTDYLM(G/A)₀₋₂₀ |
| SEQ ID NO: 373(B9-V10): | (G/A)₀₋₂₀LPHAI(X0)RLDL(X)GRDLTDYLM(G/A)₀₋₂₀ |
| SEQ ID NO: 374(B9-V11): | (G/A)₀₋₂₀LPHAI(X0)RLDLA(X)RDLTDYLM(G/A)₀₋₂₀ |
| SEQ ID NO: 375(B9-V12): | (G/A)₀₋₂₀LPHAI(X0)RLDLAG(X)DLTDYLM(G/A)₀₋₂₀ |
| SEQ ID NO: 376(B9-V13): | (G/A)₀₋₂₀LPHAI(X0)RLDLAGR(X)LTDYLM(G/A)₀₋₂₀ |
| SEQ ID NO: 377(B9-V14): | (G/A)₀₋₂₀LPHAI(X0)RLDLAGRD(X)TDYLM(G/A)₀₋₂₀ |
| SEQ ID NO: 378(B9-V15): | (G/A)₀₋₂₀LPHAI(X0)RLDLAGRDL(X)DYLM(G/A)₀₋₂₀ |
| SEQ ID NO: 379(B9-V16): | (G/A)₀₋₂₀LPHAI(X0)RLDLAGRDLT(X)YLM(G/A)₀₋₂₀ |
| SEQ ID NO: 380(B9-V17): | (G/A)₀₋₂₀LPHAI(X0)RLDLAGRDLTD(X)LM(G/A)₀₋₂₀ |
| SEQ ID NO: 381(B9-V18): | (G/A)₀₋₂₀LPHAI(X0)RLDLAGRDLTDY(X)M(G/A)₀₋₂₀ |
| SEQ ID NO: 382(B9-V19): | (G/A)₀₋₂₀LPHAI(X0)RLDLAGRDLTDYL(X)(G/A)₀₋₂₀ |
| SEQ ID NO: 383 N9-M1): | (G/A)₀₋₂₀V(X0)DSGDGVTH(X0)VPIYEGYAL(G/A)₀₋₂₀ |
| SEQ ID NO: 384(N9-M2): | (G/A)₀₋₂₀(X0)DSGDGVTH(X0)VPIYEGYALP(G/A)₀₋₂₀ |
| SEQ ID NO: 385(N9-M3): | (G/A)₀₋₂₀DSGDGVTH(X0)VPIYEGYALPH(G/A)₀₋₂₀ |
| SEQ ID NO: 386(N9-M4): | (G/A)₀₋₂₀SGDGVTH(X0)VPIYEGYALPHA(G/A)₀₋₂₀ |
| SEQ ID NO: 387(N9-M5): | (G/A)₀₋₂₀GDGVTH(X0)VPIYEGYALPHAI(G/A)₀₋₂₀ |
| SEQ ID NO: 388(N9-M6): | (G/A)₀₋₂₀DGVTH(X0)VPIYEGYALPHAI(X0)(G/A)₀₋₂₀ |
| SEQ ID NO: 389(N9-M7): | (G/A)₀₋₂₀GVTH(X0)VPIYEGYALPHAI(X0)R(G/A)₀₋₂₀ |
| SEQ ID NO: 390(N9-M8): | (G/A)₀₋₂₀VTH(X0)VPIYEGYALPHAI(X0)RL(G/A)₀₋₂₀ |
| SEQ ID NO: 391(N9-M9): | (G/A)₀₋₂₀TH(X0)VPIYEGYALPHAI(X0)RLD(G/A)₀₋₂₀ |
| SEQ ID NO: 392(N9): | (G/A)₀₋₂₀H(X0)VPIYEGYALPHAI(X0)RLDL(G/A)₀₋₂₀ |
| SEQ ID NO: 393(N9-P1): | (G/A)₀₋₂₀(X0)VPIYEGYALPHAI(X0)RLDLA(G/A)₀₋₂₀ |
| SEQ ID NO: 394(N9-P2): | (G/A)₀₋₂₀VPIYEGYALPHAI(X0)RLDLAG(G/A)₀₋₂₀ |
| SEQ ID NO: 395(N9-P3): | (G/A)₀₋₂₀PIYEGYALPHAI(X0)RLDLAGR(G/A)₀₋₂₀ |
| SEQ ID NO: 396(N9-P4): | (G/A)₀₋₂₀IYEGYALPHAI(X0)RLDLAGRD(G/A)₀₋₂₀ |
| SEQ ID NO: 397(N9-P5): | (G/A)₀₋₂₀YEGYALPHAI(X0)RLDLAGRDL(G/A)₀₋₂₀ |
| SEQ ID NO: 398(N9-P6): | (G/A)₀₋₂₀EGYALPHAI(X0)RLDLAGRDLT(G/A)₀₋₂₀ |
| SEQ ID NO: 399(N9-P7): | (G/A)₀₋₂₀GYALPHAI(X0)RLDLAGRDLTD(G/A)₀₋₂₀ |
| SEQ ID NO: 400(N9-P8): | (G/A)₀₋₂₀YALPHAI(X0)RLDLAGRDLTDY(G/A)₀₋₂₀ |
| SEQ ID NO: 401(N9-P9): | (G/A)₀₋₂₀ALPHAI(X0)RLDLAGRDLTDYL(G/A)₀₋₂₀ |
| SEQ ID NO: 402(N9-P10): | (G/A)₀₋₂₀LPHAI(X0)RLDLAGRDLTDYLM(G/A)₀₋₂₀ |
| SEQ ID NO: 403(N9-P11): | (G/A)₀₋₂₀PHAI(X0)RLDLAGRDLTDYLMK(G/A)₀₋₂₀ |
| SEQ ID NO: 404(N9-P12): | (G/A)₀₋₂₀HAI(X0)RLDLAGRDLTDYLMKI(G/A)₀₋₂₀ |
| SEQ ID NO: 405(N9-P13): | (G/A)₀₋₂₀AI(X0)RLDLAGRDLTDYLMKIL(G/A)₀₋₂₀ |
| SEQ ID NO: 406(B9-P04): | (G/A)₀₋₂₀I(X0)RLDLAGRDLTDYLMKILT(G/A)₀₋₂₀ |
| SEQ ID NO: 407(B9-P05): | (G/A)₀₋₂₀(X0)RLDLAGRDLTDYLMKILTE(G/A)₀₋₂₀ |
| SEQ ID NO: 408(B9-P06): | (G/A)₀₋₂₀(X0)LDLAGRDLTDYLMKILTER(G/A)₀₋₂₀ |
| SEQ ID NO: 409(B9-P07): | (G/A)₀₋₂₀(X0)DLAGRDLTDYLMKILTERG(G/A)₀₋₂₀ |
| SEQ ID NO: 410(B9-P08): | (G/A)₀₋₂₀(X0)LAGRDLTDYLMKILTERGY(G/A)₀₋₂₀ |
| SEQ ID NO: 411(B9-P09): | (G/A)₀₋₂₀(X0)AGRDLTDYLMKILTERGYS(G/A)₀₋₂₀ |
| SEQ ID NO: 412(B9-P10): | (G/A)₀₋₂₀(X0)GRDLTDYLMKILTERGYSF(G/A)₀₋₂₀ |
| SEQ ID NO: 413(B9-NV01): | (G/A)₀₋₂₀(X)PHAI(X0)RLDLAGRDLTDYLM(G/A)₀₋₂₀ |
| SEQ ID NO: 414(B9-NV02): | (G/A)₀₋₂₀L(X)HAI(X0)RLDLAGRDLTDYLM(G/A)₀₋₂₀ |
| SEQ ID NO: 415(B9-NV03): | (G/A)₀₋₂₀LP(X)AI(X0)RLDLAGRDLTDYLM(G/A)₀₋₂₀ |
| SEQ ID NO: 416(B9-NV04): | (G/A)₀₋₂₀LPH(X)I(X0)RLDLAGRDLTDYLM(G/A)₀₋₂₀ |
| SEQ ID NO: 417(B9-NV05): | (G/A)₀₋₂₀LPHA(X)(X0)RLDLAGRDLTDYLM(G/A)₀₋₂₀ |
| SEQ ID NO: 418(B9-NV06): | (G/A)₀₋₂₀LPHA(X)(X0)RLDLAGRDLTDYLM(G/A)₀₋₂₀ |
| SEQ ID NO: 419(B9-NV07): | (G/A)₀₋₂₀LPHAI(X0)(X)LDLAGRDLTDYLM(G/A)₀₋₂₀ |
| SEQ ID NO: 420(B9-NV08): | (G/A)₀₋₂₀LPHAI(X0)R(X)DLAGRDLTDYLM(G/A)₀₋₂₀ |
| SEQ ID NO: 421(B9-NV09): | (G/A)₀₋₂₀LPHAI(X0)RL(X)LAGRDLTDYLM(G/A)₀₋₂₀ |
| SEQ ID NO: 422(B9-NV10): | (G/A)₀₋₂₀LPHAI(X0)RLD(X)AGRDLTDYLM(G/A)₀₋₂₀ |
| SEQ ID NO: 423(B9-NV11): | (G/A)₀₋₂₀LPHAI(X0)RLDL(X)GRDLTDYLM(G/A)₀₋₂₀ |
| SEQ ID NO: 424(B9-NV12): | (G/A)₀₋₂₀LPHAI(X0)RLDLA(X)RDLTDYLM(G/A)₀₋₂₀ |
| SEQ ID NO: 425(B9-NV13): | (G/A)₀₋₂₀LPHAI(X0)RLDLAG(X)DLTDYLM(G/A)₀₋₂₀ |
| SEQ ID NO: 426(B9-NV14): | (G/A)₀₋₂₀LPHAI(X0)RLDLAGR(X)LTDYLM(G/A)₀₋₂₀ |
| SEQ ID NO: 427(B9-NV15): | (G/A)₀₋₂₀LPHAI(X0)RLDLAGRD(X)TDYLM(G/A)₀₋₂₀ |
| SEQ ID NO: 428(B9-NV16): | (G/A)₀₋₂₀LPHAI(X0)RLDLAGRDL(X)DYLM(G/A)₀₋₂₀ |
| SEQ ID NO: 429(B9-NV17): | (G/A)₀₋₂₀LPHAI(X0)RLDLAGRDLT(X)YLM(G/A)₀₋₂₀ |
| SEQ ID NO: 430(B9-NV18): | (G/A)₀₋₂₀LPHAI(X0)RLDLAGRDLTD(X)LM(G/A)₀₋₂₀ |
| SEQ ID NO: 431(B9-NV19): | (G/A)₀₋₂₀LPHAI(X0)RLDLAGRDLTDY(X)M(G/A)₀₋₂₀ |
| SEQ ID NO: 432(B9-NV20): | (G/A)₀₋₂₀LPHAI(X0)RLDLAGRDLTDYL(X)(G/A)₀₋₂₀ |
| SEQ ID NO: 433 (N9-X1): | (G/A)₀₋₂₀(X)(X0)DSGDGVTH(X0)VPIYEGYALPHAI(X0)RLDLAGRDLTDYLMKI(G/A)₀₋₂₀ |
| SEQ ID NO: 434 (N9-X2): | (G/A)₀₋₂₀V(X0)(X)SGDGVTH(X0)VPIYEGYALPHAI(X0)RLDLAGRDLTDYLMKI(G/A)₀₋₂₀ |
| SEQ ID NO: 435 (N9-X3): | (G/A)₀₋₂₀V(X0)D(X)GDGVTH(X0)VPIYEGYALPHAI(X0)RLDLAGRDLTDYLMKI(G/A)₀₋₂₀ |
| SEQ ID NO: 436 (N9-X4): | (G/A)₀₋₂₀V(X0)DS(X)DGVTH(X0)VPIYEGYALPHAI(X0)RLDLAGRDLTDYLMKI(G/A)₀₋₂₀ |
| SEQ ID NO: 437 (N9-X5): | (G/A)₀₋₂₀V(X0)DSG(X)GVTH(X0)VPIYEGYALPHAI(X0)RLDLAGRDLTDYLMKI(G/A)₀₋₂₀ |
| SEQ ID NO: 438 (N9-X6): | (G/A)₀₋₂₀V(X0)DSGD(X)VTH(X0)VPIYEGYALPHAI(X0)RLDLAGRDLTDYLMKI(G/A)₀₋₂₀ |
| SEQ ID NO: 439 (N9-X7): | (G/A)₀₋₂₀V(X0)DSGDG(X)TH(X0)VPIYEGYALPHAI(X0)RLDLAGRDLTDYLMKI(G/A)₀₋₂₀ |
| SEQ ID NO: 440 (N9-X8): | (G/A)₀₋₂₀V(X0)DSGDGV(X)H(X0)VPIYEGYALPHAI(X0)RLDLAGRDLTDYLMKI(G/A)₀₋₂₀ |
| SEQ ID NO: 441 (N9-X9): | (G/A)₀₋₂₀V(X0)DSGDGVT(X)(X0)VPIYEGYALPHAI(X0)RLDLAGRDLTDYLMKI(G/A)₀₋₂₀ |
| SEQ ID NO: 442(N9-X10): | (G/A)₀₋₂₀V(X0)DSGDGVTH(X0)(X)PIYEGYALPHAI(X0)RLDLAGRDLTDYLMKI(G/A)₀₋₂₀ |
| SEQ ID NO: 443(N9-X11): | (G/A)₀₋₂₀V(X0)DSGDGVTH(X0)V(X)IYEGYALPHAI(X0)RLDLAGRDLTDYLMKI(G/A)₀₋₂₀ |
| SEQ ID NO: 444(N9-X12): | (G/A)₀₋₂₀V(X0)DSGDGVTH(X0)VP(X)YEGYALPHAI(X0)RLDLAGRDLTDYLMKI(G/A)₀₋₂₀ |
| SEQ ID NO: 445(N9-X13): | (G/A)₀₋₂₀V(X0)DSGDGVTH(X0)VPI(X)EGYALPHAI(X0)RLDLAGRDLTDYLMKI(G/A)₀₋₂₀ |
| SEQ ID NO: 446(N9-X14): | (G/A)₀₋₂₀V(X0)DSGDGVTH(X0)VPIY(X)GYALPHAI(X0)RLDLAGRDLTDYLMKI(G/A)₀₋₂₀ |
| SEQ ID NO: 447(N9-X15): | (G/A)₀₋₂₀V(X0)DSGDGVTH(X0)VPIYE(X)YALPHAI(X0)RLDLAGRDLTDYLMKI(G/A)₀₋₂₀ |
| SEQ ID NO: 448(N9-X16): | (G/A)₀₋₂₀V(X0)DSGDGVTH(X0)VPIYEG(X)ALPHAI(X0)RLDLAGRDLTDYLMKI(G/A)₀₋₂₀ |
| SEQ ID NO: 449(N9-X17): | (G/A)₀₋₂₀V(X0)DSGDGVTH(X0)VPIYEGY(X)LPHAI(X0)RLDLAGRDLTDYLMKI(G/A)₀₋₂₀ |
| SEQ ID NO: 450(N9-X18): | (G/A)₀₋₂₀V(X0)DSGDGVTH(X0)VPIYEGYA(X)PHAI(X0)RLDLAGRDLTDYLMKI(G/A)₀₋₂₀ |
| SEQ ID NO: 451(N9-X19): | (G/A)₀₋₂₀V(X0)DSGDGVTH(X0)VPIYEGYAL(X)HAI(X0)RLDLAGRDLTDYLMKI(G/A)₀₋₂₀ |
| SEQ ID NO: 452(N9-X20): | (G/A)₀₋₂₀V(X0)DSGDGVTH(X0)VPIYEGYALP(X)AI(X0)RLDLAGRDLTDYLMKI(G/A)₀₋₂₀ |
| SEQ ID NO: 453(N9-X21): | (G/A)₀₋₂₀V(X0)DSGDGVTH(X0)VPIYEGYALPH(X)I(X0)RLDLAGRDLTDYLMKI(G/A)₀₋₂₀ |
| SEQ ID NO: 454(N9-X22): | (G/A)₀₋₂₀V(X0)DSGDGVTH(X0)VPIYEGYALPHA(X)(X0)RLDLAGRDLTDYLMKI(G/A)₀₋₂₀ |
| SEQ ID NO: 455(N9-X23): | (G/A)₀₋₂₀V(X0)DSGDGVTH(X0)VPIYEGYALPHAI(X0)(X)LDLAGRDLTDYLMKI(G/A)₀₋₂₀ |
| SEQ ID NO: 456(N9-X24): | (G/A)₀₋₂₀V(X0)DSGDGVTH(X0)VPIYEGYALPHAI(X0)R(X)DLAGRDLTDYLMKI(G/A)₀₋₂₀ |
| SEQ ID NO: 457(N9-X25): | (G/A)₀₋₂₀V(X0)DSGDGVTH(X0)VPIYEGYALPHAI(X0)RL(X)LAGRDLTDYLMKI(G/A)₀₋₂₀ |
| SEQ ID NO: 458(N9-X26): | (G/A)₀₋₂₀V(X0)DSGDGVTH(X0)VPIYEGYALPHAI(X0)RLD(X)AGRDLTDYLMKI(G/A)₀₋₂₀ |
| SEQ ID NO: 459(N9-X27): | (G/A)₀₋₂₀V(X0)DSGDGVTH(X0)VPIYEGYALPHAI(X0)RLDL(X)GRDLTDYLMKI(G/A)₀₋₂₀ |
| SEQ ID NO: 460(N9-X28): | (G/A)₀₋₂₀V(X0)DSGDGVTH(X0)VPIYEGYALPHAI(X0)RLDLA(X)RDLTDYLMKI(G/A)₀₋₂₀ |
| SEQ ID NO: 461(N9-X29): | (G/A)₀₋₂₀V(X0)DSGDGVTH(X0)VPIYEGYALPHAI(X0)RLDLAG(X)DLTDYLMKI(G/A)₀₋₂₀ |
| SEQ ID NO: 462(N9-X30): | (G/A)₀₋₂₀V(X0)DSGDGVTH(X0)VPIYEGYALPHAI(X0)RLDLAGR(X)LTDYLMKI(G/A)₀₋₂₀ |
| SEQ ID NO: 463(N9-X31): | (G/A)₀₋₂₀V(X0)DSGDGVTH(X0)VPIYEGYALPHAI(X0)RLDLAGRD(X)TDYLMKI(G/A)₀₋₂₀ |
| SEQ ID NO: 464(N9-X32): | (G/A)₀₋₂₀V(X0)DSGDGVTH(X0)VPIYEGYALPHAI(X0)RLDLAGRDL(X)DYLMKI(G/A)₀₋₂₀ |
| SEQ ID NO: 465(N9-X33): | (G/A)₀₋₂₀V(X0)DSGDGVTH(X0)VPIYEGYALPHAI(X0)RLDLAGRDLT(X)YLMKI(G/A)₀₋₂₀ |
| SEQ ID NO: 466(N9-X34): | (G/A)₀₋₂₀V(X0)DSGDGVTH(X0)VPIYEGYALPHAI(X0)RLDLAGRDLTD(X)LMKI(G/A)₀₋₂₀ |
| SEQ ID NO: 467(N9-X35): | (G/A)₀₋₂₀V(X0)DSGDGVTH(X0)VPIYEGYALPHAI(X0)RLDLAGRDLTDY(X)MKI(G/A)₀₋₂₀ |
| SEQ ID NO: 468(N9-X36): | (G/A)₀₋₂₀V(X0)DSGDGVTH(X0)VPIYEGYALPHAI(X0)RLDLAGRDLTDYL(X)KI(G/A)₀₋₂₀ |
| SEQ ID NO: 469(N9-X37): | (G/A)₀₋₂₀V(X0)DSGDGVTH(X0)VPIYEGYALPHAI(X0)RLDLAGRDLTDYLM(X)I(G/A)₀₋₂₀ |
| SEQ ID NO: 470(N9-X38): | (G/A)₀₋₂₀V(X0)DSGDGVTH(X0)VPIYEGYALPHAI(X0)RLDLAGRDLTDYLMK(X)(G/A)₀₋₂₀ |
| SEQ ID NO: 471(NB10-core): | (X)₀₋₂₀AGRDLTDYLM(X)₀₋₂₀ |
| SEQ ID NO: 472(NB10-core): | (G/A)₀₋₂₀(X)GRDLTDYLM(G/A)₀₋₂₀ |
| SEQ ID NO: 473(NB10-core): | (G/A)₀₋₂₀A(X)RDLTDYLM(G/A)₀₋₂₀ |
| SEQ ID NO: 474(NB10-core): | (G/A)₀₋₂₀AG(X)DLTDYLM(G/A)₀₋₂₀ |
| SEQ ID NO: 475(NB10-core): | (G/A)₀₋₂₀AGR(X)LTDYLM(G/A)₀₋₂₀ |
| SEQ ID NO: 476(NB10-core): | (G/A)₀₋₂₀AGRD(X)TDYLM(G/A)₀₋₂₀ |
| SEQ ID NO: 477(NB10-core): | (G/A)₀₋₂₀AGRDL(X)DYLM(G/A)₀₋₂₀ |
| SEQ ID NO: 478(NB10-core): | (G/A)₀₋₂₀AGRDLT(X)YLM(G/A)₀₋₂₀ |
| SEQ ID NO: 479(NB10-core): | (G/A)₀₋₂₀AGRDLTD(X)LM(G/A)₀₋₂₀ |
| SEQ ID NO: 480(NB10-core): | (G/A)₀₋₂₀AGRDLTDY(X)M(G/A)₀₋₂₀ |
| SEQ ID NO: 481(NB10-core): | (G/A)₀₋₂₀AGRDLTDYL(X)(G/A)₀₋₂₀ |
| SEQ ID NO: 482(N10-NV1): | (G/A)₀₋₂₀(X)DYLMKILTE(G/A)₀₋₂₀ |
| SEQ ID NO: 483(N10-NV2): | (G/A)₀₋₂₀T(X)YLMKILTE(G/A)₀₋₂₀ |
| SEQ ID NO: 484(N10-NV3): | (G/A)₀₋₂₀TD(X)LMKILTE(G/A)₀₋₂₀ |
| SEQ ID NO: 485(N10-NV4): | (G/A)₀₋₂₀TDY(X)MKILTE(G/A)₀₋₂₀ |
| SEQ ID NO: 486(N10-NV5): | (G/A)₀₋₂₀TDYL(X)KILTE(G/A)₀₋₂₀ |
| SEQ ID NO: 487(N10-NV6): | (G/A)₀₋₂₀TDYLM(X)ILTE(G/A)₀₋₂₀ |
| SEQ ID NO: 488(N10-NV7): | (G/A)₀₋₂₀TDYLMK(X)LTE(G/A)₀₋₂₀ |
| SEQ ID NO: 489(N10-NV8): | (G/A)₀₋₂₀TDYLMKI(X)TE(G/A)₀₋₂₀ |
| SEQ ID NO: 490(N10-NV9): | (G/A)₀₋₂₀TDYLMKIL(X)E(G/A)₀₋₂₀ |
| SEQ ID NO: 491(N10-NV10): | (G/A)₀₋₂₀TDYLMKILT(X)(G/A)₀₋₂₀ |
| SEQ ID NO: 492(N10-N1): | (G/A)₀₋₂₀LPHAI(X0)RLDL(G/A)₀₋₂₀ |
| SEQ ID NO: 493(N10-N2): | (G/A)₀₋₂₀PHAI(X0)RLDLA(G/A)₀₋₂₀ |
| SEQ ID NO: 494(N10-N3): | (G/A)₀₋₂₀HAI(X0)RLDLAG(G/A)₀₋₂₀ |
| SEQ ID NO: 495(N10-N4): | (G/A)₀₋₂₀AI(X0)RLDLAGR(G/A)₀₋₂₀ |
| SEQ ID NO: 496(N10-N5): | (G/A)₀₋₂₀I(X0)RLDLAGRD(G/A)₀₋₂₀ |
| SEQ ID NO: 497(N10-N6): | (G/A)₀₋₂₀(X0)RLDLAGRDL(G/A)₀₋₂₀ |
| SEQ ID NO: 498(N10-N7): | (G/A)₀₋₂₀(X0)LDLAGRDLT(G/A)₀₋₂₀ |
| SEQ ID NO: 499(N10-N8): | (G/A)₀₋₂₀(X0)DLAGRDLTD(G/A)₀₋₂₀ |
| SEQ ID NO: 500(N10-N9): | (G/A)₀₋₂₀(X0)LAGRDLTDY(G/A)₀₋₂₀ |
| SEQ ID NO: 501(N10-N10): | (G/A)₀₋₂₀(X0)AGRDLTDYL(G/A)₀₋₂₀ |
| SEQ ID NO: 502(N10-N11): | (G/A)₀₋₂₀(X0)GRDLTDYLM(G/A)₀₋₂₀ |
| SEQ ID NO: 503(N10-N12): | (G/A)₀₋₂₀(X0)RDLTDYLMK(G/A)₀₋₂₀ |
| SEQ ID NO: 504(N10-N13): | (G/A)₀₋₂₀(X0)DLTDYLMKI(G/A)₀₋₂₀ |
| SEQ ID NO: 505(N10-N14): | (G/A)₀₋₂₀(X0)LTDYLMKIL(G/A)₀₋₂₀ |
| SEQ ID NO: 506(N10-N15): | (G/A)₀₋₂₀(X0)TDYLMKILT(G/A)₀₋₂₀ |
| SEQ ID NO: 507(N10-N16): | (G/A)₀₋₂₀(X0)DYLMKILTE(G/A)₀₋₂₀ |
| SEQ ID NO: 508(N10-N17): | (G/A)₀₋₂₀(X0)YLMKILTER(G/A)₀₋₂₀ |
| SEQ ID NO: 509(N10-N18): | (G/A)₀₋₂₀(X0)LMKILTERG(G/A)₀₋₂₀ |
| SEQ ID NO: 510(N10-N19): | (G/A)₀₋₂₀(X0)MKILTERGY(G/A)₀₋₂₀ |
| SEQ ID NO: 511(N10-N20): | (G/A)₀₋₂₀(X0)KILTERGYS(G/A)₀₋₂₀ |
| SEQ ID NO: 512(N10-N21): | (G/A)₀₋₂₀(X0)ILTERGYSF(X0)TTAEREIVR(G/A)₀₋₂₀ |
| SEQ ID NO: 513(N10-N22): | (G/A)₀₋₂₀ILTERGYSF(X0)(G/A)₀₋₂₀ |
| SEQ ID NO: 514(N10-N23): | (G/A)₀₋₂₀LTERGYSF(X0)T(G/A)₀₋₂₀ |
| SEQ ID NO: 515(N10-N24): | (G/A)₀₋₂₀TERGYSF(X0)TT(G/A)₀₋₂₀ |
| SEQ ID NO: 516(N10-N25): | (G/A)₀₋₂₀ERGYSF(X0)TTA(G/A)₀₋₂₀ |
| SEQ ID NO: 517(N10-N26): | (G/A)₀₋₂₀RGYSF(X0)TTAE(G/A)₀₋₂₀ |
| SEQ ID NO: 518(N10-N27): | (G/A)₀₋₂₀GYSF(X0)TTAER(G/A)₀₋₂₀ |
| SEQ ID NO: 519(N10-N28): | (G/A)₀₋₂₀YSF(X0)TTAERE(G/A)₀₋₂₀ |
| SEQ ID NO: 520(N10-N29): | (G/A)₀₋₂₀SF(X0)TTAEREI(G/A)₀₋₂₀ |
| SEQ ID NO: 521(N10-N30): | (G/A)₀₋₂₀F(X0)TTAEREIV(G/A)₀₋₂₀ |
| SEQ ID NO: 522(N10-N31): | (G/A)₀₋₂₀(X0)TTAEREIVR(G/A)₀₋₂₀ |
| SEQ ID NO: 523(N10-V1): | (G/A)₀₋₂₀(X)GRDLTDYLMKILTERGYSF(G/A)₀₋₂₀ |
| SEQ ID NO: 524(N10-V2): | (G/A)₀₋₂₀A(X)RDLTDYLMKILTERGYSF(G/A)₀₋₂₀ |
| SEQ ID NO: 525(N10-V3): | (G/A)₀₋₂₀AG(X)DLTDYLMKILTERGYSF(G/A)₀₋₂₀ |
| SEQ ID NO: 526(N10-V4): | (G/A)₀₋₂₀AGR(X)LTDYLMKILTERGYSF(G/A)₀₋₂₀ |
| SEQ ID NO: 527(N10-V5): | (G/A)₀₋₂₀AGRD(X)TDYLMKILTERGYSF(G/A)₀₋₂₀ |
| SEQ ID NO: 528(N10-V6): | (G/A)₀₋₂₀AGRDL(X)DYLMKILTERGYSF(G/A)₀₋₂₀ |
| SEQ ID NO: 529(N10-V7): | (G/A)₀₋₂₀AGRDLT(X)YLMKILTERGYSF(G/A)₀₋₂₀ |
| SEQ ID NO: 530(N10-V8): | (G/A)₀₋₂₀AGRDLTD(X)LMKILTERGYSF(G/A)₀₋₂₀ |
| SEQ ID NO: 531(N10-V9): | (G/A)₀₋₂₀AGRDLTDY(X)MKILTERGYSF(G/A)₀₋₂₀ |
| SEQ ID NO: 532(N10-V10): | (G/A)₀₋₂₀AGRDLTDYL(X)KILTERGYSF(G/A)₀₋₂₀ |
| SEQ ID NO: 533(N10-V11): | (G/A)₀₋₂₀AGRDLTDYLM(X)ILTERGYSF(G/A)₀₋₂₀ |
| SEQ ID NO: 534(N10-V12): | (G/A)₀₋₂₀AGRDLTDYLMK(X)LTERGYSF(G/A)₀₋₂₀ |
| SEQ ID NO: 535(N10-V13): | (G/A)₀₋₂₀AGRDLTDYLMKI(X)TERGYSF(G/A)₀₋₂₀ |
| SEQ ID NO: 536(N10-V14): | (G/A)₀₋₂₀AGRDLTDYLMKIL(X)ERGYSF(G/A)₀₋₂₀ |
| SEQ ID NO: 537(N10-V15): | (G/A)₀₋₂₀AGRDLTDYLMKILT(X)RGYSF(G/A)₀₋₂₀ |
| SEQ ID NO: 538(N10-V16): | (G/A)₀₋₂₀AGRDLTDYLMKILTE(X)GYSF(G/A)₀₋₂₀ |
| SEQ ID NO: 539(N10-V17): | (G/A)₀₋₂₀AGRDLTDYLMKILTER(X)YSF(G/A)₀₋₂₀ |
| SEQ ID NO: 540(N10-V18): | (G/A)₀₋₂₀AGRDLTDYLMKILTERG(X)SF(G/A)₀₋₂₀ |
| SEQ ID NO: 541(N10-V19): | (G/A)₀₋₂₀AGRDLTDYLMKILTERGY(X)F(G/A)₀₋₂₀ |
| SEQ ID NO: 542(N10-V20): | (G/A)₀₋₂₀AGRDLTDYLMKILTERGYS(X)(G/A)₀₋₂₀ |
| SEQ ID NO: 543(N10-M1): | (G/A)₀₋₂₀LPHAI(X0)RLDLAGRDLTDYLM(G/A)₀₋₂₀ |
| SEQ ID NO: 544(N10-M2): | (G/A)₀₋₂₀PHAI(X0)RLDLAGRDLTDYLMK(G/A)₀₋₂₀ |
| SEQ ID NO: 545(N10-M3): | (G/A)₀₋₂₀HAI(X0)RLDLAGRDLTDYLMKI(G/A)₀₋₂₀ |
| SEQ ID NO: 546(N10-M4): | (G/A)₀₋₂₀AI(X0)RLDLAGRDLTDYLMKIL(G/A)₀₋₂₀ |
| SEQ ID NO: 547(N10-M5): | (G/A)₀₋₂₀I(X0)RLDLAGRDLTDYLMKILT(G/A)₀₋₂₀ |
| SEQ ID NO: 548(N10-M6): | (G/A)₀₋₂₀(X0)RLDLAGRDLTDYLMKILTE(G/A)₀₋₂₀ |
| SEQ ID NO: 549(N10-M7): | (G/A)₀₋₂₀RLDLAGRDLTDYLMKILTER(G/A)₀₋₂₀ |
| SEQ ID NO: 550(N10-M8): | (G/A)₀₋₂₀LDLAGRDLTDYLMKILTERG(G/A)₀₋₂₀ |
| SEQ ID NO: 551(N10-M9): | (G/A)₀₋₂₀DLAGRDLTDYLMKILTERGY(G/A)₀₋₂₀ |
| SEQ ID NO: 552(N10-M10): | (G/A)₀₋₂₀LAGRDLTDYLMKILTERGYS(G/A)₀₋₂₀ |
| SEQ ID NO: 553(N10): | (G/A)₀₋₂₀AGRDLTDYLMKILTERGYSF(G/A)₀₋₂₀ |
| SEQ ID NO: 554(N10-P1): | (G/A)₀₋₂₀GRDLTDYLMKILTERGYSF(X0)(G/A)₀₋₂₀ |
| SEQ ID NO: 555(N10-P2): | (G/A)₀₋₂₀RDLTDYLMKILTERGYSF(X0)T(G/A)₀₋₂₀ |
| SEQ ID NO: 556(N10-P3): | (G/A)₀₋₂₀DLTDYLMKILTERGYSF(X0)TT(G/A)₀₋₂₀ |
| SEQ ID NO: 557(N10-P4): | (G/A)₀₋₂₀LTDYLMKILTERGYSF(X0)TTA(G/A)₀₋₂₀ |
| SEQ ID NO: 558(N10-P5): | (G/A)₀₋₂₀TDYLMKILTERGYSF(X0)TTAE(G/A)₀₋₂₀ |
| SEQ ID NO: 559(N10-P6): | (G/A)₀₋₂₀DYLMKILTERGYSF(X0)TTAER(G/A)₀₋₂₀ |
| SEQ ID NO: 560(N10-P7): | (G/A)₀₋₂₀YLMKILTERGYSF(X0)TTAERE(G/A)₀₋₂₀ |
| SEQ ID NO: 561(N10-P8): | (G/A)₀₋₂₀LMKILTERGYSF(X0)TTAEREI(G/A)₀₋₂₀ |
| SEQ ID NO: 562(N10-P9): | (G/A)₀₋₂₀MKILTERGYSF(X0)TTAEREIV(G/A)₀₋₂₀ |
| SEQ ID NO: 563(N10-P10): | (G/A)₀₋₂₀KILTERGYSF(X0)TTAEREIVR(G/A)₀₋₂₀ |
| SEQ ID NO: 564(N10-X1): | (G/A)₀₋₂₀(X)PHAI(X0)RLDLAGRDLTDYLMKILTERGYSF(X0)TTAEREIVR(G/A)₀₋₂₀ |
| SEQ ID NO: 565(N10-X2): | (G/A)₀₋₂₀L(X)HAI(X0)RLDLAGRDLTDYLMKILTERGYSF(X0)TTAEREIVR(G/A)₀₋₂₀ |
| SEQ ID NO: 566(N10-X3): | (G/A)₀₋₂₀LP(X)AI(X0)RLDLAGRDLTDYLMKILTERGYSF(X0)TTAEREIVR(G/A)₀₋₂₀ |
| SEQ ID NO: 567(N10-X4): | (G/A)₀₋₂₀LPH(X)I(X0)RLDLAGRDLTDYLMKILTERGYSF(X0)TTAEREIVR(G/A)₀₋₂₀ |
| SEQ ID NO: 568(N10-X5): | (G/A)₀₋₂₀LPHA(X)(X0)RLDLAGRDLTDYLMKILTERGYSF(X0)TTAEREIVR(G/A)₀₋₂₀ |
| SEQ ID NO: 569(N10-X6): | (G/A)₀₋₂₀LPHAI(X0)(X)LDLAGRDLTDYLMKILTERGYSF(X0)TTAEREIVR(G/A)₀₋₂₀ |
| SEQ ID NO: 570(N10-X7): | (G/A)₀₋₂₀LPHAI(X0)R(X)DLAGRDLTDYLMKILTERGYSF(X0)TTAEREIVR(G/A)₀₋₂₀ |
| SEQ ID NO: 571(N10-X8): | (G/A)₀₋₂₀LPHAI(X0)RL(X)LAGRDLTDYLMKILTERGYSF(X0)TTAEREIVR(G/A)₀₋₂₀ |
| SEQ ID NO: 572(N10-X9): | (G/A)₀₋₂₀LPHAI(X0)RLD(X)AGRDLTDYLMKILTERGYSF(X0)TTAEREIVR(G/A)₀₋₂₀ |
| SEQ ID NO: 573(N10-X10): | (G/A)₀₋₂₀LPHAI(X0)RLDL(X)GRDLTDYLMKILTERGYSF(X0)TTAEREIVR(G/A)₀₋₂₀ |
| SEQ ID NO: 574(N10-X11): | (G/A)₀₋₂₀LPHAI(X0)RLDLA(X)RDLTDYLMKILTERGYSF(X0)TTAEREIVR(G/A)₀₋₂₀ |
| SEQ ID NO: 575(N10-X12): | (G/A)₀₋₂₀LPHAI(X0)RLDLAG(X)DLTDYLMKILTERGYSF(X0)TTAEREIVR(G/A)₀₋₂₀ |
| SEQ ID NO: 576(N10-X13): | (G/A)₀₋₂₀LPHAI(X0)RLDLAGR(X)LTDYLMKILTERGYSF(X0)TTAEREIVR(G/A)₀₋₂₀ |
| SEQ ID NO: 577(N10-X14): | (G/A)₀₋₂₀LPHAI(X0)RLDLAGRD(X)TDYLMKILTERGYSF(X0)TTAEREIVR(G/A)₀₋₂₀ |
| SEQ ID NO: 578(N10-X15): | (G/A)₀₋₂₀LPHAI(X0)RLDLAGRDL(X)DYLMKILTERGYSF(X0)TTAEREIVR(G/A)₀₋₂₀ |
| SEQ ID NO: 579(N10-X16): | (G/A)₀₋₂₀LPHAI(X0)RLDLAGRDLT(X)YLMKILTERGYSF(X0)TTAEREIVR(G/A)₀₋₂₀ |
| SEQ ID NO: 580(N10-X17): | (G/A)₀₋₂₀LPHAI(X0)RLDLAGRDLTD(X)LMKILTERGYSF(X0)TTAEREIVR(G/A)₀₋₂₀ |
| SEQ ID NO: 581(N10-X18): | (G/A)₀₋₂₀LPHAI(X0)RLDLAGRDLTDY(X)MKILTERGYSF(X0)TTAEREIVR(G/A)₀₋₂₀ |
| SEQ ID NO: 582(N10-X19): | (G/A)₀₋₂₀LPHAI(X0)RLDLAGRDLTDYL(X)KILTERGYSF(X0)TTAEREIVR(G/A)₀₋₂₀ |
| SEQ ID NO: 583(N10-X20): | (G/A)₀₋₂₀LPHAI(X0)RLDLAGRDLTDYLM(X)ILTERGYSF(X0)TTAEREIVR(G/A)₀₋₂₀ |
| SEQ ID NO: 584(N10-X21): | (G/A)₀₋₂₀LPHAI(X0)RLDLAGRDLTDYLMK(X)LTERGYSF(X0)TTAEREIVR(G/A)₀₋₂₀ |
| SEQ ID NO: 585(N10-X22): | (G/A)₀₋₂₀LPHAI(X0)RLDLAGRDLTDYLMKI(X)TERGYSF(X0)TTAEREIVR(G/A)₀₋₂₀ |
| SEQ ID NO: 586(N10-X23): | (G/A)₀₋₂₀LPHAI(X0)RLDLAGRDLTDYLMKIL(X)ERGYSF(X0)TTAEREIVR(G/A)₀₋₂₀ |
| SEQ ID NO: 587(N10-X24): | (G/A)₀₋₂₀LPHAI(X0)RLDLAGRDLTDYLMKILT(X)RGYSF(X0)TTAEREIVR(G/A)₀₋₂₀ |
| SEQ ID NO: 588(N10-X25): | (G/A)₀₋₂₀LPHAI(X0)RLDLAGRDLTDYLMKILTE(X)GYSF(X0)TTAEREIVR(G/A)₀₋₂₀ |
| SEQ ID NO: 589(N10-X26): | (G/A)₀₋₂₀LPHAI(X0)RLDLAGRDLTDYLMKILTER(X)YSF(X0)TTAEREIVR(G/A)₀₋₂₀ |
| SEQ ID NO: 590(N10-X27): | (G/A)₀₋₂₀LPHAI(X0)RLDLAGRDLTDYLMKILTERG(X)SF(X0)TTAEREIVR(G/A)₀₋₂₀ |
| SEQ ID NO: 591(N10-X28): | (G/A)₀₋₂₀LPHAI(X0)RLDLAGRDLTDYLMKILTERGY(X)F(X0)TTAEREIVR(G/A)₀₋₂₀ |
| SEQ ID NO: 592(N10-X29): | (G/A)₀₋₂₀LPHAI(X0)RLDLAGRDLTDYLMKILTERGYS(X)(X0)TTAEREIVR(G/A)₀₋₂₀ |
| SEQ ID NO: 593(N10-X30): | (G/A)₀₋₂₀LPHAI(X0)RLDLAGRDLTDYLMKILTERGYSF(X0)(X)TAEREIVR(G/A)₀₋₂₀ |
| SEQ ID NO: 594(N10-X31): | (G/A)₀₋₂₀LPHAI(X0)RLDLAGRDLTDYLMKILTERGYSF(X0)T(X)AEREIVR(G/A)₀₋₂₀ |
| SEQ ID NO: 595(N10-X32): | (G/A)₀₋₂₀LPHAI(X0)RLDLAGRDLTDYLMKILTERGYSF(X0)TT(X)EREIVR(G/A)₀₋₂₀ |
| SEQ ID NO: 596(N10-X33): | (G/A)₀₋₂₀LPHAI(X0)RLDLAGRDLTDYLMKILTERGYSF(X0)TTA(X)REIVR(G/A)₀₋₂₀ |
| SEQ ID NO: 597(N10-X34): | (G/A)₀₋₂₀LPHAI(X0)RLDLAGRDLTDYLMKILTERGYSF(X0)TTAE(X)EIVR(G/A)₀₋₂₀ |
| SEQ ID NO: 598(N10-X35): | (G/A)₀₋₂₀LPHAI(X0)RLDLAGRDLTDYLMKILTERGYSF(X0)TTAER(X)IVR(G/A)₀₋₂₀ |
| SEQ ID NO: 599(N10-X36): | (G/A)₀₋₂₀LPHAI(X0)RLDLAGRDLTDYLMKILTERGYSF(X0)TTAERE(X)VR(G/A)₀₋₂₀ |
| SEQ ID NO: 600(N10-X37): | (G/A)₀₋₂₀LPHAI(X0)RLDLAGRDLTDYLMKILTERGYSF(X0)TTAEREI(X)R(G/A)₀₋₂₀ |
| SEQ ID NO: 601(N10-X38): | (G/A)₀₋₂₀LPHAI(X0)RLDLAGRDLTDYLMKILTERGYSF(X0)TTAEREIV(X)(G/A)₀₋₂₀ |
| SEQ ID NO: 602(B11-core): | (X)₀₋₂₀LCYVALDFEQ(X)₀₋₂₀ |
| SEQ ID NO: 603(B11-NV01): | (G/A)₀₋₂₀(X)CYVALDFE(X0)(G/A)₀₋₂₀ |
| SEQ ID NO: 604(B11-NV02): | (G/A)₀₋₂₀L(X)YVALDFE(X0)(G/A)₀₋₂₀ |
| SEQ ID NO: 605(B11-NV03): | (G/A)₀₋₂₀LC(X)VALDFE(X0)(G/A)₀₋₂₀ |
| SEQ ID NO: 606(B11-NV04): | (G/A)₀₋₂₀LCY(X)ALDFE(X0)(G/A)₀₋₂₀ |
| SEQ ID NO: 607(B11-NV05): | (G/A)₀₋₂₀LCYV(X)LDFE(X0)(G/A)₀₋₂₀ |
| SEQ ID NO: 608(B11-NV06): | (G/A)₀₋₂₀LCYVA(X)DFE(X0)(G/A)₀₋₂₀ |
| SEQ ID NO: 609(B11-NV07): | (G/A)₀₋₂₀LCYVAL(X)FE(X0)(G/A)₀₋₂₀ |
| SEQ ID NO: 610(B11-NV08): | (G/A)₀₋₂₀LCYVALD(X)E(X0)(G/A)₀₋₂₀ |
| SEQ ID NO: 611(B11-NV09): | (G/A)₀₋₂₀LCYVALDF(X)(X0)(G/A)₀₋₂₀ |
| SEQ ID NO: 612(B11-T01): | (G/A)₀₋₂₀F(X0)TTAEREIV(G/A)₀₋₂₀ |
| SEQ ID NO: 613(B11-T02): | (G/A)₀₋₂₀(X0)TTAEREIVR(G/A)₀₋₂₀ |
| SEQ ID NO: 614(B11-T03): | (G/A)₀₋₂₀(X0)TAEREIVRD(G/A)₀₋₂₀ |
| SEQ ID NO: 615(B11-T04): | (G/A)₀₋₂₀(X0)AEREIVRDI(G/A)₀₋₂₀ |
| SEQ ID NO: 616(B11-T05): | (G/A)₀₋₂₀(X0)EREIVRDIK(G/A)₀₋₂₀ |
| SEQ ID NO: 617(B11-T06): | (G/A)₀₋₂₀(X0)REIVRDIKE(G/A)₀₋₂₀ |
| SEQ ID NO: 618(B11-T07): | (G/A)₀₋₂₀(X0)EIVRDIKEK(G/A)₀₋₂₀ |
| SEQ ID NO: 619(B11-T08): | (G/A)₀₋₂₀(X0)IVRDIKEKL(G/A)₀₋₂₀ |
| SEQ ID NO: 620(B11-T09): | (G/A)₀₋₂₀(X0)VRDIKEKLC(G/A)₀₋₂₀ |
| SEQ ID NO: 621(B11-T10): | (G/A)₀₋₂₀(X0)RDIKEKLCY(G/A)₀₋₂₀ |
| SEQ ID NO: 622(B11-T11): | (G/A)₀₋₂₀(X0)DIKEKLCYV(G/A)₀₋₂₀ |
| SEQ ID NO: 623(B11-T12): | (G/A)₀₋₂₀(X0)IKEKLCYVA(G/A)₀₋₂₀ |
| SEQ ID NO: 624(B11-T13): | (G/A)₀₋₂₀(X0)KEKLCYVAL(G/A)₀₋₂₀ |
| SEQ ID NO: 625(B11-T14): | (G/A)₀₋₂₀(X0)EKLCYVALD(G/A)₀₋₂₀ |
| SEQ ID NO: 626(B11-T15): | (G/A)₀₋₂₀(X0)KLCYVALDF(G/A)₀₋₂₀ |
| SEQ ID NO: 627(B11-T16): | (G/A)₀₋₂₀(X0)LCYVALDFE(G/A)₀₋₂₀ |
| SEQ ID NO: 628(B11-T17): | (G/A)₀₋₂₀LCYVALDFE(X0)(G/A)₀₋₂₀ |
| SEQ ID NO: 629(B11-T18): | (G/A)₀₋₂₀CYVALDFE(X0)E(G/A)₀₋₂₀ |
| SEQ ID NO: 630(B11-T19): | (G/A)₀₋₂₀YVALDFE(X0)EM(G/A)₀₋₂₀ |
| SEQ ID NO: 631(B11-T20): | (G/A)₀₋₂₀VALDFE(X0)EMA(G/A)₀₋₂₀ |
| SEQ ID NO: 632(B11-T21): | (G/A)₀₋₂₀ALDFE(X0)EMAT(G/A)₀₋₂₀ |
| SEQ ID NO: 633(B11-T22): | (G/A)₀₋₂₀LDFE(X0)EMATA(G/A)₀₋₂₀ |
| SEQ ID NO: 634(B11-T23): | (G/A)₀₋₂₀DFE(X0)EMATAA(G/A)₀₋₂₀ |
| SEQ ID NO: 635(B11-T24): | (G/A)₀₋₂₀FE(X0)EMATAAS(G/A)₀₋₂₀ |
| SEQ ID NO: 636(B11-T25): | (G/A)₀₋₂₀E(X0)EMATAASS(G/A)₀₋₂₀ |
| SEQ ID NO: 637(B11-T26): | (G/A)₀₋₂₀(X0)EMATAASSS(G/A)₀₋₂₀ |
| SEQ ID NO: 638(B11-T27): | (G/A)₀₋₂₀(X0)MATAASSSS(G/A)₀₋₂₀ |
| SEQ ID NO: 639(B11-T28): | (G/A)₀₋₂₀(X0)ATAASSSSL(G/A)₀₋₂₀ |
| SEQ ID NO: 640(B11-T29): | (G/A)₀₋₂₀(X0)TAASSSSLE(G/A)₀₋₂₀ |
| SEQ ID NO: 641(B11-T30): | (G/A)₀₋₂₀(X0)AASSSSLEK(G/A)₀₋₂₀ |
| SEQ ID NO: 642(B11-T31): | (G/A)₀₋₂₀(X0)ASSSSLEKS(G/A)₀₋₂₀ |
| SEQ ID NO: 643(B11-T32): | (G/A)₀₋₂₀(X0)SSSSLEKSY(G/A)₀₋₂₀ |
| SEQ ID NO: 644(B11-M01): | (G/A)₀₋₂₀F(X0)TTAEREIVRDIKEKLCYV(G/A)₀₋₂₀ |
| SEQ ID NO: 645(B11-M02): | (G/A)₀₋₂₀(X0)TTAEREIVRDIKEKLCYVA(G/A)₀₋₂₀ |
| SEQ ID NO: 646(B11-M03): | (G/A)₀₋₂₀(X0)TAEREIVRDIKEKLCYVAL(G/A)₀₋₂₀ |
| SEQ ID NO: 647(B11-M04): | (G/A)₀₋₂₀(X0)AEREIVRDIKEKLCYVALD(G/A)₀₋₂₀ |
| SEQ ID NO: 648(B11-M05): | (G/A)₀₋₂₀(X0)EREIVRDIKEKLCYVALDF(G/A)₀₋₂₀ |
| SEQ ID NO: 649(B11-M06): | (G/A)₀₋₂₀(X0)REIVRDIKEKLCYVALDFE(G/A)₀₋₂₀ |
| SEQ ID NO: 650(B11-M07): | (G/A)₀₋₂₀EIVRDIKEKLCYVALDFE(X0)(G/A)₀₋₂₀ |
| SEQ ID NO: 651(B11-M08): | (G/A)₀₋₂₀IVRDIKEKLCYVALDFE(X0)EM(G/A)₀₋₂₀ |
| SEQ ID NO: 652(B11-M09): | (G/A)₀₋₂₀VRDIKEKLCYVALDFE(X0)EMA(G/A)₀₋₂₀ |
| SEQ ID NO: 653(B11-M10): | (G/A)₀₋₂₀RDIKEKLCYVALDFE(X0)EMAT(G/A)₀₋₂₀ |
| SEQ ID NO: 654(B11): | (G/A)₀₋₂₀DIKEKLCYVALDFE(X0)EMATA(G/A)₀₋₂₀ |
| SEQ ID NO: 655(B11-P01): | (G/A)₀₋₂₀IKEKLCYVALDFE(X0)EMATAA(G/A)₀₋₂₀ |
| SEQ ID NO: 656(B11-P02): | (G/A)₀₋₂₀KEKLCYVALDFE(X0)EMATAAS(G/A)₀₋₂₀ |
| SEQ ID NO: 657(B11-P03): | (G/A)₀₋₂₀EKLCYVALDFE(X0)EMATAASS(G/A)₀₋₂₀ |
| SEQ ID NO: 658(B11-P04): | (G/A)₀₋₂₀KLCYVALDFE(X0)EMATAASSS(G/A)₀₋₂₀ |
| SEQ ID NO: 659(B11-P05): | (G/A)₀₋₂₀LCYVALDFE(X0)EMATAASSSS(G/A)₀₋₂₀ |
| SEQ ID NO: 660(B11-P06): | (G/A)₀₋₂₀CYVALDFE(X0)EMATAASSSSL(G/A)₀₋₂₀ |
| SEQ ID NO: 661(B11-P07): | (G/A)₀₋₂₀YVALDFE(X0)EMATAASSSSLE(G/A)₀₋₂₀ |
| SEQ ID NO: 662(B11-P08): | (G/A)₀₋₂₀VALDFE(X0)EMATAASSSSLEK(G/A)₀₋₂₀ |
| SEQ ID NO: 663(B11-P09): | (G/A)₀₋₂₀ALDFE(X0)EMATAASSSSLEKS(G/A)₀₋₂₀ |
| SEQ ID NO: 664(B11-P10): | (G/A)₀₋₂₀LDFE(X0)EMATAASSSSLEKSY(G/A)₀₋₂₀ |
| SEQ ID NO: 665(B11-P11): | (G/A)₀₋₂₀DFE(X0)EMATAASSSSLEKSYE(G/A)₀₋₂₀ |
| SEQ ID NO: 666(B11-V01): | (G/A)₀₋₂₀(X)IKEKLCYVALDFE(X0)EMATA(G/A)₀₋₂₀ |
| SEQ ID NO: 667(B11-V02): | (G/A)₀₋₂₀D(X)KEKLCYVALDFE(X0)EMATA(G/A)₀₋₂₀ |
| SEQ ID NO: 668(B11-V03): | (G/A)₀₋₂₀DI(X)EKLCYVALDFE(X0)EMATA(G/A)₀₋₂₀ |
| SEQ ID NO: 669(B11-V04): | (G/A)₀₋₂₀DIK(X)KLCYVALDFE(X0)EMATA(G/A)₀₋₂₀ |
| SEQ ID NO: 670(B11-V05): | (G/A)₀₋₂₀DIKE(X)LCYVALDFE(X0)EMATA(G/A)₀₋₂₀ |
| SEQ ID NO: 671(B11-V06): | (G/A)₀₋₂₀DIKEK(X)CYVALDFE(X0)EMATA(G/A)₀₋₂₀ |
| SEQ ID NO: 672(B11-V07): | (G/A)₀₋₂₀DIKEKL(X)YVALDFE(X0)EMATA(G/A)₀₋₂₀ |
| SEQ ID NO: 673(B11-V08): | (G/A)₀₋₂₀DIKEKLC(X)VALDFE(X0)EMATA(G/A)₀₋₂₀ |
| SEQ ID NO: 674(B11-V09): | (G/A)₀₋₂₀DIKEKLCY(X)ALDFE(X0)EMATA(G/A)₀₋₂₀ |
| SEQ ID NO: 675(B11-V10): | (G/A)₀₋₂₀DIKEKLCYV(X)LDFE(X0)EMATA(G/A)₀₋₂₀ |
| SEQ ID NO: 676(B11-V11): | (G/A)₀₋₂₀DIKEKLCYVA(X)DFE(X0)EMATA(G/A)₀₋₂₀ |
| SEQ ID NO: 677(B11-V12): | (G/A)₀₋₂₀DIKEKLCYVAL(X)FE(X0)EMATA(G/A)₀₋₂₀ |
| SEQ ID NO: 678(B11-V13): | (G/A)₀₋₂₀DIKEKLCYVALD(X)E(X0)EMATA(G/A)₀₋₂₀ |
| SEQ ID NO: 679(B11-V14): | (G/A)₀₋₂₀DIKEKLCYVALDF(X)(X0)EMATA(G/A)₀₋₂₀ |
| SEQ ID NO: 680(B11-V15): | (G/A)₀₋₂₀DIKEKLCYVALDFE(X0)(X)MATA(G/A)₀₋₂₀ |
| SEQ ID NO: 681(B11-V16): | (G/A)₀₋₂₀DIKEKLCYVALDFE(X0)E(X)ATA(G/A)₀₋₂₀ |
| SEQ ID NO: 682(B11-V17): | (G/A)₀₋₂₀DIKEKLCYVALDFE(X0)EM(X)TA(G/A)₀₋₂₀ |
| SEQ ID NO: 683(B11-V18): | (G/A)₀₋₂₀DIKEKLCYVALDFE(X0)EMA(X)A(G/A)₀₋₂₀ |
| SEQ ID NO: 684(B11-V19): | (G/A)₀₋₂₀DIKEKLCYVALDFE(X0)EMAT(X)(G/A)₀₋₂₀ |
| SEQ ID NO: 685(B11-X01): | (G/A)₀₋₂₀(X)(X0)TTAEREIVRDIKEKLCYVALDFE(X0)EMATAASSSSLEKSY(G/A)₀₋₂₀ |
| SEQ ID NO: 686(B11-X02): | (G/A)₀₋₂₀F(X0)(X)TAEREIVRDIKEKLCYVALDFE(X0)EMATAASSSSLEKSY(G/A)₀₋₂₀ |
| SEQ ID NO: 687(B11-X03): | (G/A)₀₋₂₀F(X0)T(X)AEREIVRDIKEKLCYVALDFE(X0)EMATAASSSSLEKSY(G/A)₀₋₂₀ |
| SEQ ID NO: 688(B11-X04): | (G/A)₀₋₂₀F(X0)TT(X)EREIVRDIKEKLCYVALDFE(X0)EMATAASSSSLEKSY(G/A)₀₋₂₀ |
| SEQ ID NO: 689(B11-X05): | (G/A)₀₋₂₀F(X0)TTA(X)REIVRDIKEKLCYVALDFE(X0)EMATAASSSSLEKSY(G/A)₀₋₂₀ |
| SEQ ID NO: 690(B11-X06): | (G/A)₀₋₂₀F(X0)TTAE(X)EIVRDIKEKLCYVALDFE(X0)EMATAASSSSLEKSY(G/A)₀₋₂₀ |
| SEQ ID NO: 691(B11-X07): | (G/A)₀₋₂₀F(X0)TTAER(X)IVRDIKEKLCYVALDFE(X0)EMATAASSSSLEKSY(G/A)₀₋₂₀ |
| SEQ ID NO: 692(B11-X08): | (G/A)₀₋₂₀F(X0)TTAERE(X)VRDIKEKLCYVALDFE(X0)EMATAASSSSLEKSY(G/A)₀₋₂₀ |
| SEQ ID NO: 693(B11-X09): | (G/A)₀₋₂₀F(X0)TTAEREI(X)RDIKEKLCYVALDFE(X0)EMATAASSSSLEKSY(G/A)₀₋₂₀ |
| SEQ ID NO: 694(B11-X10): | (G/A)₀₋₂₀F(X0)TTAEREIV(X)DIKEKLCYVALDFE(X0)EMATAASSSSLEKSY(G/A)₀₋₂₀ |
| SEQ ID NO: 695(B11-X11): | (G/A)₀₋₂₀F(X0)TTAEREIVR(X)IKEKLCYVALDFE(X0)EMATAASSSSLEKSY(G/A)₀₋₂₀ |
| SEQ ID NO: 696(B11-X12): | (G/A)₀₋₂₀F(X0)TTAEREIVRD(X)KEKLCYVALDFE(X0)EMATAASSSSLEKSY(G/A)₀₋₂₀ |
| SEQ ID NO: 697(B11-X13): | (G/A)₀₋₂₀F(X0)TTAEREIVRDI(X)EKLCYVALDFE(X0)EMATAASSSSLEKSY(G/A)₀₋₂₀ |
| SEQ ID NO: 698(B11-X14): | (G/A)₀₋₂₀F(X0)TTAEREIVRDIK(X)KLCYVALDFE(X0)EMATAASSSSLEKSY(G/A)₀₋₂₀ |
| SEQ ID NO: 699(B11-X15): | (G/A)₀₋₂₀F(X0)TTAEREIVRDIKE(X)LCYVALDFE(X0)EMATAASSSSLEKSY(G/A)₀₋₂₀ |
| SEQ ID NO: 700(B11-X16): | (G/A)₀₋₂₀F(X0)TTAEREIVRDIKEK(X)CYVALDFE(X0)EMATAASSSSLEKSY(G/A)₀₋₂₀ |
| SEQ ID NO: 701(B11-X17): | (G/A)₀₋₂₀F(X0)TTAEREIVRDIKEKL(X)YVALDFE(X0)EMATAASSSSLEKSY(G/A)₀₋₂₀ |
| SEQ ID NO: 702(B11-X18): | (G/A)₀₋₂₀F(X0)TTAEREIVRDIKEKLC(X)VALDFE(X0)EMATAASSSSLEKSY(G/A)₀₋₂₀ |
| SEQ ID NO: 703(B11-X19): | (G/A)₀₋₂₀F(X0)TTAEREIVRDIKEKLCY(X)ALDFE(X0)EMATAASSSSLEKSY(G/A)₀₋₂₀ |
| SEQ ID NO: 704(B11-X20): | (G/A)₀₋₂₀F(X0)TTAEREIVRDIKEKLCYV(X)LDFE(X0)EMATAASSSSLEKSY(G/A)₀₋₂₀ |
| SEQ ID NO: 705(B11-X21): | (G/A)₀₋₂₀F(X0)TTAEREIVRDIKEKLCYVA(X)DFE(X0)EMATAASSSSLEKSY(G/A)₀₋₂₀ |
| SEQ ID NO: 706(B11-X22): | (G/A)₀₋₂₀F(X0)TTAEREIVRDIKEKLCYVAL(X)FE(X0)EMATAASSSSLEKSY(G/A)₀₋₂₀ |
| SEQ ID NO: 707(B11-X23): | (G/A)₀₋₂₀F(X0)TTAEREIVRDIKEKLCYVALD(X)E(X0)EMATAASSSSLEKSY(G/A)₀₋₂₀ |
| SEQ ID NO: 708(B11-X24): | (G/A)₀₋₂₀F(X0)TTAEREIVRDIKEKLCYVALDF(X)(X0)EMATAASSSSLEKSY(G/A)₀₋₂₀ |
| SEQ ID NO: 709(B11-X25): | (G/A)₀₋₂₀F(X0)TTAEREIVRDIKEKLCYVALDFE(X0)(X)MATAASSSSLEKSY(G/A)₀₋₂₀ |
| SEQ ID NO: 710(B11-X26): | (G/A)₀₋₂₀F(X0)TTAEREIVRDIKEKLCYVALDFE(X0)E(X)ATAASSSSLEKSY(G/A)₀₋₂₀ |
| SEQ ID NO: 711(B11-X27): | (G/A)₀₋₂₀F(X0)TTAEREIVRDIKEKLCYVALDFE(X0)EM(X)TAASSSSLEKSY(G/A)₀₋₂₀ |
| SEQ ID NO: 712(B11-X28): | (G/A)₀₋₂₀F(X0)TTAEREIVRDIKEKLCYVALDFE(X0)EMA(X)AASSSSLEKSY(G/A)₀₋₂₀ |
| SEQ ID NO: 713(B11-X29): | (G/A)₀₋₂₀F(X0)TTAEREIVRDIKEKLCYVALDFE(X0)EMAT(X)ASSSSLEKSY(G/A)₀₋₂₀ |
| SEQ ID NO: 714(B11-X30): | (G/A)₀₋₂₀F(X0)TTAEREIVRDIKEKLCYVALDFE(X0)EMATA(X)SSSSLEKSY(G/A)₀₋₂₀ |
| SEQ ID NO: 715(B11-X31): | (G/A)₀₋₂₀F(X0)TTAEREIVRDIKEKLCYVALDFE(X0)EMATAS(X)SSSLEKSY(G/A)₀₋₂₀ |
| SEQ ID NO: 716(B11-X32): | (G/A)₀₋₂₀F(X0)TTAEREIVRDIKEKLCYVALDFE(X0)EMATASS(X)SSLEKSY(G/A)₀₋₂₀ |
| SEQ ID NO: 717(B11-X33): | (G/A)₀₋₂₀F(X0)TTAEREIVRDIKEKLCYVALDFE(X0)EMATASSS(X)SLEKSY(G/A)₀₋₂₀ |
| SEQ ID NO: 718(B11-X34): | (G/A)₀₋₂₀F(X0)TTAEREIVRDIKEKLCYVALDFE(X0)EMATASSSS(X)LEKSY(G/A)₀₋₂₀ |
| SEQ ID NO: 719(B11-X35): | (G/A)₀₋₂₀F(X0)TTAEREIVRDIKEKLCYVALDFE(X0)EMATAASSSS(X)EKSY(G/A)₀₋₂₀ |
| SEQ ID NO: 720(B11-X36): | (G/A)₀₋₂₀F(X0)TTAEREIVRDIKEKLCYVALDFE(X0)EMATAASSSSL(X)KSY(G/A)₀₋₂₀ |
| SEQ ID NO: 721(B11-X37): | (G/A)₀₋₂₀F(X0)TTAEREIVRDIKEKLCYVALDFE(X0)EMATAASSSSLE(X)SY(G/A)₀₋₂₀ |
| SEQ ID NO: 722(B11-X38): | (G/A)₀₋₂₀F(X0)TTAEREIVRDIKEKLCYVALDFE(X0)EMATAASSSSLEK(X)Y(G/A)₀₋₂₀ |
| SEQ ID NO: 723(B11-X39): | (G/A)₀₋₂₀F(X0)TTAEREIVRDIKEKLCYVALDFE(X0)EMATAASSSSLEKS(X)(G/A)₀₋₂₀ |
| SEQ ID NO: 724(B17-core): | (X)₀₋₂₀KYSVWIGGSI(X)₀₋₂₀ |
| SEQ ID NO: 725(B17-TV01): | (G/A)₀₋₂₀(X)YSVWIGGSI(G/A)₀₋₂₀ |
| SEQ ID NO: 726(B17-TV02): | (G/A)₀₋₂₀K(X)SVWIGGSI(G/A)₀₋₂₀ |
| SEQ ID NO: 727(B17-TV03): | (G/A)₀₋₂₀KY(X)VWIGGSI(G/A)₀₋₂₀ |
| SEQ ID NO: 728(B17-TV04): | (G/A)₀₋₂₀KYS(X)WIGGSI(G/A)₀₋₂₀ |
| SEQ ID NO: 729(B17-TV05): | (G/A)₀₋₂₀KYSV(X)IGGSI(G/A)₀₋₂₀ |
| SEQ ID NO: 730(B17-TV06): | (G/A)₀₋₂₀KYSVW(X)GGSI(G/A)₀₋₂₀ |
| SEQ ID NO: 731(B17-TV07): | (G/A)₀₋₂₀KYSVWI(X)GSI(G/A)₀₋₂₀ |
| SEQ ID NO: 732(B17-TV08): | (G/A)₀₋₂₀KYSVWIG(X)SI(G/A)₀₋₂₀ |
| SEQ ID NO: 733(B17-TV09): | (G/A)₀₋₂₀KYSVWIGG(X)I(G/A)₀₋₂₀ |
| SEQ ID NO: 734(B17-TV10): | (G/A)₀₋₂₀KYSVWIGGS(X)(G/A)₀₋₂₀ |
| SEQ ID NO: 735(B17-T01): | (G/A)₀₋₂₀(X0)PSTMKIKII(G/A)₀₋₂₀ |
| SEQ ID NO: 736(B17-T02): | (G/A)₀₋₂₀(X0)STMKIKIIA(G/A)₀₋₂₀ |
| SEQ ID NO: 737(B17-T03): | (G/A)₀₋₂₀(X0)TMKIKIIAP(G/A)₀₋₂₀ |
| SEQ ID NO: 738(B17-T04): | (G/A)₀₋₂₀(X0)MKIKIIAPP(G/A)₀₋₂₀ |
| SEQ ID NO: 739(B17-T05): | (G/A)₀₋₂₀(X0)KIKIIAPPE(G/A)₀₋₂₀ |
| SEQ ID NO: 740(B17-T06): | (G/A)₀₋₂₀(X0)IKIIAPPER(G/A)₀₋₂₀ |
| SEQ ID NO: 741(B17-T07): | (G/A)₀₋₂₀(X0)KIIAPPERK(G/A)₀₋₂₀ |
| SEQ ID NO: 742(B17-T08): | (G/A)₀₋₂₀(X0)IIAPPERKY(G/A)₀₋₂₀ |
| SEQ ID NO: 743(B17-T09): | (G/A)₀₋₂₀(X0)IAPPERKYS(G/A)₀₋₂₀ |
| SEQ ID NO: 744(B17-T10): | (G/A)₀₋₂₀(X0)APPERKYSV(G/A)₀₋₂₀ |
| SEQ ID NO: 745(B17-T11): | (G/A)₀₋₂₀(X0)PPERKYSVW(G/A)₀₋₂₀ |
| SEQ ID NO: 746(B17-T12): | (G/A)₀₋₂₀(X0)PERKYSVWI(G/A)₀₋₂₀ |
| SEQ ID NO: 747(B17-T13): | (G/A)₀₋₂₀(X0)ERKYSVWIG(G/A)₀₋₂₀ |
| SEQ ID NO: 748(B17-T14): | (G/A)₀₋₂₀(X0)RKYSVWIGG(G/A)₀₋₂₀ |
| SEQ ID NO: 749(B17-T15): | (G/A)₀₋₂₀(X0)KYSVWIGGS(G/A)₀₋₂₀ |
| SEQ ID NO: 750(B17-T16): | (G/A)₀₋₂₀(X0)YSVWIGGSI(G/A)₀₋₂₀ |
| SEQ ID NO: 751(B17-T17): | (G/A)₀₋₂₀(X0)SVWIGGSIL(G/A)₀₋₂₀ |
| SEQ ID NO: 752(B17-T18): | (G/A)₀₋₂₀(X0)VWIGGSILA(G/A)₀₋₂₀ |
| SEQ ID NO: 753(B17-T19): | (G/A)₀₋₂₀(X0)WIGGSILAS(G/A)₀₋₂₀ |
| SEQ ID NO: 754(B17-T20): | (G/A)₀₋₂₀(X0)IGGSILASL(G/A)₀₋₂₀ |
| SEQ ID NO: 755(B17-T21): | (G/A)₀₋₂₀(X0)GGSILASLS(G/A)₀₋₂₀ |
| SEQ ID NO: 756(B17-T22): | (G/A)₀₋₂₀(X0)GSILASLST(G/A)₀₋₂₀ |
| SEQ ID NO: 757(B17-T23): | (G/A)₀₋₂₀(X0)SILASLSTF(G/A)₀₋₂₀ |
| SEQ ID NO: 758(B17-T24): | (G/A)₀₋₂₀(X0)ILASLSTFQ(G/A)₀₋₂₀ |
| SEQ ID NO: 759(B17-T25): | (G/A)₀₋₂₀(X0)LASLSTFQQ(G/A)₀₋₂₀ |
| SEQ ID NO: 760(B17-T26): | (G/A)₀₋₂₀(X0)ASLSTFQQM(G/A)₀₋₂₀ |
| SEQ ID NO: 761(B17-T27): | (G/A)₀₋₂₀(X0)SLSTFQQMW(G/A)₀₋₂₀ |
| SEQ ID NO: 762(B17-T28): | (G/A)₀₋₂₀(X0)LSTFQQMWI(G/A)₀₋₂₀ |
| SEQ ID NO: 763(B17-T29): | (G/A)₀₋₂₀LSTFQQMWI(X0)(G/A)₀₋₂₀ |
| SEQ ID NO: 764(B17-T30): | (G/A)₀₋₂₀STFQQMWI(X0)K(G/A)₀₋₂₀ |
| SEQ ID NO: 765(B17-T31): | (G/A)₀₋₂₀TFQQMWI(X0)KQ(G/A)₀₋₂₀ |
| SEQ ID NO: 766(B17-M01): | (G/A)₀₋₂₀APSTMKIKIIAPPERKYSVW(G/A)₀₋₂₀ |
| SEQ ID NO: 767(B17-M02): | (G/A)₀₋₂₀PSTMKIKIIAPPERKYSVWI(G/A)₀₋₂₀ |
| SEQ ID NO: 768(B17-M03): | (G/A)₀₋₂₀STMKIKIIAPPERKYSVWIG(G/A)₀₋₂₀ |
| SEQ ID NO: 769(B17-M04): | (G/A)₀₋₂₀TMKIKIIAPPERKYSVWIGG(G/A)₀₋₂₀ |
| SEQ ID NO: 770(B17-M05): | (G/A)₀₋₂₀MKIKIIAPPERKYSVWIGGS(G/A)₀₋₂₀ |
| SEQ ID NO: 771(B17-M06): | (G/A)₀₋₂₀KIKIIAPPERKYSVWIGGSI(G/A)₀₋₂₀ |
| SEQ ID NO: 772(B17-M07): | (G/A)₀₋₂₀IKIIAPPERKYSVWIGGSIL(G/A)₀₋₂₀ |
| SEQ ID NO: 773(B17-M08): | (G/A)₀₋₂₀KIIAPPERKYSVWIGGSILA(G/A)₀₋₂₀ |
| SEQ ID NO: 774(B17-M09): | (G/A)₀₋₂₀IIAPPERKYSVWIGGSILAS(G/A)₀₋₂₀ |
| SEQ ID NO: 775(B17-M10): | (G/A)₀₋₂₀IAPPERKYSVWIGGSILASL(G/A)₀₋₂₀ |
| SEQ ID NO: 776(B17): | (G/A)₀₋₂₀APPERKYSVWIGGSILASLS(G/A)₀₋₂₀ |
| SEQ ID NO: 777(B17-P01): | (G/A)₀₋₂₀PPERKYSVWIGGSILASLST(G/A)₀₋₂₀ |
| SEQ ID NO: 778(B17-P02): | (G/A)₀₋₂₀PERKYSVWIGGSILASLSTF(G/A)₀₋₂₀ |
| SEQ ID NO: 779(B17-P03): | (G/A)₀₋₂₀ERKYSVWIGGSILASLSTFQ(G/A)₀₋₂₀ |
| SEQ ID NO: 780(B17-P04): | (G/A)₀₋₂₀RKYSVWIGGSILASLSTFQQ(G/A)₀₋₂₀ |
| SEQ ID NO: 781(B17-P05): | (G/A)₀₋₂₀KYSVWIGGSILASLSTFQQM(G/A)₀₋₂₀ |
| SEQ ID NO: 782(B17-P06): | (G/A)₀₋₂₀YSVWIGGSILASLSTFQQMW(G/A)₀₋₂₀ |
| SEQ ID NO: 783(B17-P07): | (G/A)₀₋₂₀SVWIGGSILASLSTFQQMWI(G/A)₀₋₂₀ |
| SEQ ID NO: 784(B17-P08): | (G/A)₀₋₂₀VWIGGSILASLSTFQQMWI(X0)(G/A)₀₋₂₀ |
| SEQ ID NO: 785(B17-P09): | (G/A)₀₋₂₀WIGGSILASLSTFQQMWI(X0)K(G/A)₀₋₂₀ |
| SEQ ID NO: 786(B17-P10): | (G/A)₀₋₂₀IGGSILASLSTFQQMWI(X0)KQ(G/A)₀₋₂₀ |
| SEQ ID NO: 787(B17-V01): | (G/A)₀₋₂₀(X)PPERKYSVWIGGSILASLS(G/A)₀₋₂₀ |
| SEQ ID NO: 788(B17-V02): | (G/A)₀₋₂₀A(X)PERKYSVWIGGSILASLS(G/A)₀₋₂₀ |
| SEQ ID NO: 789(B17-V03): | (G/A)₀₋₂₀AP(X)ERKYSVWIGGSILASLS(G/A)₀₋₂₀ |
| SEQ ID NO: 790(B17-V04): | (G/A)₀₋₂₀APP(X)RKYSVWIGGSILASLS(G/A)₀₋₂₀ |
| SEQ ID NO: 791(B17-V05): | (G/A)₀₋₂₀APPE(X)KYSVWIGGSILASLS(G/A)₀₋₂₀ |
| SEQ ID NO: 792(B17-V06): | (G/A)₀₋₂₀APPER(X)YSVWIGGSILASLS(G/A)₀₋₂₀ |
| SEQ ID NO: 793(B17-V07): | (G/A)₀₋₂₀APPERK(X)SVWIGGSILASLS(G/A)₀₋₂₀ |
| SEQ ID NO: 794(B17-V08): | (G/A)₀₋₂₀APPERKY(X)VWIGGSILASLS(G/A)₀₋₂₀ |
| SEQ ID NO: 795(B17-V09): | (G/A)₀₋₂₀APPERKYS(X)WIGGSILASLS(G/A)₀₋₂₀ |
| SEQ ID NO: 796(B17-V10): | (G/A)₀₋₂₀APPERKYSV(X)IGGSILASLS(G/A)₀₋₂₀ |
| SEQ ID NO: 797(B17-V11): | (G/A)₀₋₂₀APPERKYSVW(X)GGSILASLS(G/A)₀₋₂₀ |
| SEQ ID NO: 798(B17-V12): | (G/A)₀₋₂₀APPERKYSVWI(X)GSILASLS(G/A)₀₋₂₀ |
| SEQ ID NO: 799(B17-V13): | (G/A)₀₋₂₀APPERKYSVWIG(X)SILASLS(G/A)₀₋₂₀ |
| SEQ ID NO: 800(B17-V14): | (G/A)₀₋₂₀APPERKYSVWIGG(X)ILASLS(G/A)₀₋₂₀ |
| SEQ ID NO: 801(B17-V15): | (G/A)₀₋₂₀APPERKYSVWIGGS(X)LASLS(G/A)₀₋₂₀ |
| SEQ ID NO: 802(B17-V16): | (G/A)₀₋₂₀APPERKYSVWIGGSI(X)ASLS(G/A)₀₋₂₀ |
| SEQ ID NO: 803(B17-V17): | (G/A)₀₋₂₀APPERKYSVWIGGSIL(X)SLS(G/A)₀₋₂₀ |
| SEQ ID NO: 804(B17-V18): | (G/A)₀₋₂₀APPERKYSVWIGGSILA(X)LS(G/A)₀₋₂₀ |
| SEQ ID NO: 805(B17-V19): | (G/A)₀₋₂₀APPERKYSVWIGGSILAS(X)S(G/A)₀₋₂₀ |
| SEQ ID NO: 806(B17-V20): | (G/A)₀₋₂₀APPERKYSVWIGGSILASL(X)(G/A)₀₋₂₀ |
| SEQ ID NO: 807(B17-X01): | (G/A)₀₋₂₀(X)PSTMKIKIIAPPERKYSVWIGGSILASLSTFQQMWI(X0)KQ(G/A)₀₋₂₀ |
| SEQ ID NO: 808(B17-X02): | (G/A)₀₋₂₀A(X)STMKIKIIAPPERKYSVWIGGSILASLSTFQQMWI(X0)KQ(G/A)₀₋₂₀ |
| SEQ ID NO: 809(B17-X03): | (G/A)₀₋₂₀AP(X)TMKIKIIAPPERKYSVWIGGSILASLSTFQQMWI(X0)KQ(G/A)₀₋₂₀ |
| SEQ ID NO: 810(B17-X04): | (G/A)₀₋₂₀APS(X)MKIKIIAPPERKYSVWIGGSILASLSTFQQMWI(X0)KQ(G/A)₀₋₂₀ |
| SEQ ID NO: 811(B17-X05): | (G/A)₀₋₂₀APST(X)KIKIIAPPERKYSVWIGGSILASLSTFQQMWI(X0)KQ(G/A)₀₋₂₀ |
| SEQ ID NO: 812(B17-X06): | (G/A)₀₋₂₀APSTM(X)IKIIAPPERKYSVWIGGSILASLSTFQQMWI(X0)KQ(G/A)₀₋₂₀ |
| SEQ ID NO: 813(B17-X07): | (G/A)₀₋₂₀APSTMK(X)KIIAPPERKYSVWIGGSILASLSTFQQMWI(X0)KQ(G/A)₀₋₂₀ |
| SEQ ID NO: 814(B17-X08): | (G/A)₀₋₂₀APSTMKI(X)IIAPPERKYSVWIGGSILASLSTFQQMWI(X0)KQ(G/A)₀₋₂₀ |
| SEQ ID NO: 815(B17-X09): | (G/A)₀₋₂₀APSTMKIK(X)IAPPERKYSVWIGGSILASLSTFQQMWI(X0)KQ(G/A)₀₋₂₀ |
| SEQ ID NO: 816(B17-X10): | (G/A)₀₋₂₀APSTMKIKI(X)APPERKYSVWIGGSILASLSTFQQMWI(X0)KQ(G/A)₀₋₂₀ |
| SEQ ID NO: 817(B17-X11): | (G/A)₀₋₂₀APSTMKIKII(X)PPERKYSVWIGGSILASLSTFQQMWI(X0)KQ(G/A)₀₋₂₀ |
| SEQ ID NO: 818(B17-X12): | (G/A)₀₋₂₀APSTMKIKIIA(X)PERKYSVWIGGSILASLSTFQQMWI(X0)KQ(G/A)₀₋₂₀ |
| SEQ ID NO: 819(B17-X13): | (G/A)₀₋₂₀APSTMKIKIIAP(X)ERKYSVWIGGSILASLSTFQQMWI(X0)KQ(G/A)₀₋₂₀ |
| SEQ ID NO: 820(B17-X15): | (G/A)₀₋₂₀APSTMKIKIIAPP(X)RKYSVWIGGSILASLSTFQQMWI(X0)KQ(G/A)₀₋₂₀ |
| SEQ ID NO: 821(B17-X16): | (G/A)₀₋₂₀APSTMKIKIIAPPE(X)KYSVWIGGSILASLSTFQQMWI(X0)KQ(G/A)₀₋₂₀ |
| SEQ ID NO: 822(B17-X17): | (G/A)₀₋₂₀APSTMKIKIIAPPER(X)YSVWIGGSILASLSTFQQMWI(X0)KQ(G/A)₀₋₂₀ |
| SEQ ID NO: 823(B17-X18): | (G/A)₀₋₂₀APSTMKIKIIAPPERK(X)SVWIGGSILASLSTFQQMWI(X0)KQ(G/A)₀₋₂₀ |
| SEQ ID NO: 824(B17-X19): | (G/A)₀₋₂₀APSTMKIKIIAPPERKY(X)VWIGGSILASLSTFQQMWI(X0)KQ(G/A)₀₋₂₀ |
| SEQ ID NO: 825(B17-X20): | (G/A)₀₋₂₀APSTMKIKIIAPPERKYS(X)WIGGSILASLSTFQQMWI(X0)KQ(G/A)₀₋₂₀ |
| SEQ ID NO: 826(B17-X21): | (G/A)₀₋₂₀APSTMKIKIIAPPERKYSV(X)IGGSILASLSTFQQMWI(X0)KQ(G/A)₀₋₂₀ |
| SEQ ID NO: 827(B17-X22): | (G/A)₀₋₂₀APSTMKIKIIAPPERKYSVW(X)GGSILASLSTFQQMWI(X0)KQ(G/A)₀₋₂₀ |
| SEQ ID NO: 828(B17-X23): | (G/A)₀₋₂₀APSTMKIKIIAPPERKYSVWI(X)GSILASLSTFQQMWI(X0)KQ(G/A)₀₋₂₀ |
| SEQ ID NO: 829(B17-X24): | (G/A)₀₋₂₀APSTMKIKIIAPPERKYSVWIG(X)SILASLSTFQQMWI(X0)KQ(G/A)₀₋₂₀ |
| SEQ ID NO: 830(B17-X25): | (G/A)₀₋₂₀APSTMKIKIIAPPERKYSVWIGG(X)ILASLSTFQQMWI(X0)KQ(G/A)₀₋₂₀ |
| SEQ ID NO: 831(B17-X26): | (G/A)₀₋₂₀APSTMKIKIIAPPERKYSVWIGGS(X)LASLSTFQQMWI(X0)KQ(G/A)₀₋₂₀ |
| SEQ ID NO: 832(B17-X27): | (G/A)₀₋₂₀APSTMKIKIIAPPERKYSVWIGGSI(X)ASLSTFQQMWI(X0)KQ(G/A)₀₋₂₀ |
| SEQ ID NO: 833(B17-X28): | (G/A)₀₋₂₀APSTMKIKIIAPPERKYSVWIGGSIL(X)SLSTFQQMWI(X0)KQ(G/A)₀₋₂₀ |
| SEQ ID NO: 834(B17-X29): | (G/A)₀₋₂₀APSTMKIKIIAPPERKYSVWIGGSILA(X)LSTFQQMWI(X0)KQ(G/A)₀₋₂₀ |
| SEQ ID NO: 835(B17-X30): | (G/A)₀₋₂₀APSTMKIKIIAPPERKYSVWIGGSILAS(X)STFQQMWI(X0)KQ(G/A)₀₋₂₀ |
| SEQ ID NO: 836(B17-X31): | (G/A)₀₋₂₀APSTMKIKIIAPPERKYSVWIGGSILASL(X)TFQQMWI(X0)KQ(G/A)₀₋₂₀ |
| SEQ ID NO: 837(B17-X32): | (G/A)₀₋₂₀APSTMKIKIIAPPERKYSVWIGGSILASLS(X)FQQMWI(X0)KQ(G/A)₀₋₂₀ |
| SEQ ID NO: 838(B17-X33): | (G/A)₀₋₂₀APSTMKIKIIAPPERKYSVWIGGSILASLST(X)QQMWI(X0)KQ(G/A)₀₋₂₀ |
| SEQ ID NO: 839(B17-X34): | (G/A)₀₋₂₀APSTMKIKIIAPPERKYSVWIGGSILASLSTF(X)QMWI(X0)KQ(G/A)₀₋₂₀ |
| SEQ ID NO: 840(B17-X35): | (G/A)₀₋₂₀APSTMKIKIIAPPERKYSVWIGGSILASLSTFQ(X)MWI(X0)KQ(G/A)₀₋₂₀ |
| SEQ ID NO: 841(B17-X36): | (G/A)₀₋₂₀APSTMKIKIIAPPERKYSVWIGGSILASLSTFQQ(X)WI(X0)KQ(G/A)₀₋₂₀ |
| SEQ ID NO: 842(B17-X37): | (G/A)₀₋₂₀APSTMKIKIIAPPERKYSVWIGGSILASLSTFQQM(X)I(X0)KQ(G/A)₀₋₂₀ |
| SEQ ID NO: 843(B17-X38): | (G/A)₀₋₂₀APSTMKIKIIAPPERKYSVWIGGSILASLSTFQQMW(X)(X0)KQ(G/A)₀₋₂₀ |
| SEQ ID NO: 844(B17-X39): | (G/A)₀₋₂₀APSTMKIKIIAPPERKYSVWIGGSILASLSTFQQMWI(X0)(X)Q(G/A)₀₋₂₀ |
| SEQ ID NO: 845(B17-X40): | (G/A)₀₋₂₀APSTMKIKIIAPPERKYSVWIGGSILASLSTFQQMWI(X0)K(X)(G/A)₀₋₂₀ |

In another embodiment, a peptide according to formula SEQ ID No:1 to SEQ ID No:845 is a cyclic peptide. A cyclic peptide can be made by covalently crosslinking amino acid residues together via a disulfide bond (e.g., thiol group of cysteine) or a side chain of amino acid residues (e.g., Lye, Arg, Ser, Tyr). Furthermore, a functionalized linker (e.g., polyethylene glycol or PEG) can be introduced to generate a cyclic peptide.

Although the sequences enumerated here comprise 40 AA, 20 AA, and 10 AA, it is to be understood that peptides of the invention may include any length from 5 to 40 AA.

Numerous permutations and variations of the present invention are readily apparent to a person of ordinary skill in the art in view of this disclosure. Therefore it is to be understood that the invention may be practiced otherwise than as specifically described herein.

A sequence listing is filed herewith and is incorporated by reference.

### SEQUENCE LISTING

The instant application contains a Sequence Listing which is being submitted in ASCII format via EFS-WEB and is hereby incorporated by reference in its entirety.
File name: 3_SQL
Creation date: Feb. 14, 2022
Byte size: 867,727 bytes

1. A method for the enhancing delivery of a nucleic acid into a cell, comprising introducing into a cell one or more actin-based peptides in an amount sufficient to enhance delivery, wherein the one or more actin-based peptides are selected from peptides having a sequence selected from SEQ ID No:1 to SEQ ID No:845.

2. The method of claim 1, wherein one or more actin-based peptides are introduced in the form of a composition further comprising a second molecule differing from the one or more actin-based peptides.

3. A method for enhancing cell-cell and cell-extracellular matrix adhesion, comprising treating a cell one or more actin-based peptides in an amount sufficient to enhance cell adhesion, wherein the one or more actin-based peptides are selected from peptides having a sequence selected from SEQ ID No:1 to SEQ ID No:845.

4. The method of claim 2, wherein one or more actin-based peptides are introduced in the form of a composition further comprising a second molecule differing from the one or more actin-based peptides.

5. The method of claim 2, wherein or 4, molecule is a low-molecular weight molecule or derived polymer, such as polybrene (hexadimethrine bormid), Dextrins, or PEI (polyethylenimine), or any molecule of similar nature for enhancing viral infection and exosome delivery.

6. The method of claim 2, wherein the molecule is a peptide such as cationic peptides including but not limited to Dorsocin, Histatin-5, indolicidin, LL-37, polycathonic amyloid fibrils, the Semen-Derived Enhancer of Viral Infection (SEVI), vectofusin-1, or any peptides of similar nature for enhancing viral infection and exosome delivery.

7. The method of claim 2, wherein the molecule is a peptide, such as cell-permeable peptides including but not limited to small polybasic peptides derived from proteins, for example, the third α-helix fragment of Antennapedia (Antp) homeodomain.

8. The method of claim, wherein the molecule is a protein, such as fragments of fibronectin (rFN-CH-296), or any protein of similar nature for enhancing viral infection and exosome delivery.

9. The method of claim 2, wherein the molecule is a liposome such as the phosphatidylserine (PS) liposomes, or any liposome of similar nature for delivery of nucleic acids and proteins into cells.

10. The method of claim 1, wherein the one or more actin-based peptides are incorporated into virion or exosome particles to enhance their ability to infect cells or to enter cells for the delivery of genes and proteins.

11. The method of claim 1, wherein the actin-based peptide has a core sequence selected from NB5 core sequences (SEQ ID Nos: 1 and 15-24).

12. The method of claim 1, wherein the actin-based peptide has a core sequence selected from NB7 core sequences (SEQ ID Nos: 2 and 139-148).

13. The method of claim 1, wherein the actin-based peptide has a core sequence selected from NB9 core sequences (SEQ ID Nos: 3 and 291-299).

14. The method of claim 1, wherein the actin-based peptide has a core sequence selected from NB10 core sequences (SEQ ID Nos: 4 and 471-481).

15. The method of claim 1, wherein the actin-based peptide has a core sequence selected from B11 core sequences (SEQ ID Nos: 5, 13, and 602-723).

16. The method of claim 1, wherein the actin-based peptide has a core sequence selected from B17 core sequences (SEQ ID Nos: 6, 14, and 724-845).

17. The method of claim 1, wherein the actin-based peptide has a core sequence selected from:
B5 core sequences (SEQ ID Nos: 7 and 25-138);
B6 core sequences (SEQ ID Nos: 8 and 272-290); and
B9 core sequences (SEQ ID Nos: 11, 291-299, 340-343, 364-382, and 406-432).

18. The method of claim 1, wherein the actin-based peptide has a core sequence selected from:
N7 core sequences (SEQ ID Nos: 9, 41, 149-271, 311, and 498);
N9 core sequences (SEQ ID Nos: 10, 43, 300-339, 344-363, 383-405, 433-479, and 500); and
N10 core sequences (SEQ ID Nos: 12, 44, 314, and 482-601).

19. A composition comprising a combination one or more actin-based peptides and a second molecule differing from the one or more actin-based peptides, wherein the one or more actin-based peptides are selected from peptides having a sequence selected from SEQ ID No:1 to SEQ ID No:845, and wherein the composition can enhance virus infection of cells or can enhance exosome delivery of genes and proteins into cells.

20. The composition in claim 19, wherein the molecule is selected from:
a peptide such as cationic peptides including but not limited to Dorsocin, Histatin-5, indolicidin, LL-37, polycathonic amyloid fibrils, the Semen-Derived Enhancer of Viral Infection (SEVI), vectofusin-1, or any peptides of similar nature for enhancing viral infection and exosome delivery; such as cell-permeable peptides including but not limited to small polybasic peptides derived from proteins, for example, the third α-helix fragment of Antennapedia (Antp) homeodomain; such as fragments of fibronectin (rFN-CH-296), or any protein of similar nature for enhancing viral infection and exosome delivery; or
a liposome such as the phosphatidylserine (PS) liposomes, or any liposome of similar nature for delivery of nucleic acids and proteins into cells.
